SEQUENCE LISTING (43 sequences)

SEQ ID NO: 1 - DNA, 143 bp, Homo sapiens
  CDS (27)..(140)
cctctccccttctcttccccgaggctatgtccacccggtgcggcgaggcgggcagagccagaggcacgcagccgcacaggggctacagagcccagaatcagccctacaagatgcacttaggacccccgcggctggaagaatga

SEQ ID NO: 2 - protein, 38 aa, Homo sapiens
MSTRCGEAGRARGTQPHRGYRAQNQPYKMHLGPPRLEE

SEQ ID NO: 3 - DNA, 1123 bp, Homo sapiens
  CDS (140)..(946)
cctctccccttctcttccccgaggctatgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctataggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgttgccctgaataaaagaggaaaagccaaacgagggtgcagcccccgggttaaaccccagcatatctctacccattttcttccaagattcaagcagtcggagcagccagaactttctttcacggttactgttcctgaaaagaaaaatccacctagccctatcaagtcaaagattcccctttctgcacctcggaaaaataccaactcagtgaaatacagactcaagtttcgctttggataatattaatcttggccttgtgagaaaccattctttcccctcaggagtttctataggtgtcttcagagttctgaagaaaaattactggacacagcttcagctatacttacactgtattgaagtcacgtcatttgtttcagtgtgactgaaacaaaatgttttttgataggaaggaaactg

SEQ ID NO: 4 - protein, 268 aa, Homo sapiens
MSLSFLLLLFFSHLILSAWAHGEKRLAPKGQPGPAATDRNPIGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKNPPSPIKSKIPLSAPRKNTNSVKYRLKFRFG

SEQ ID NO: 5 - DNA, 531 bp, Homo sapiens
  CDS (1)..(531)
tgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgttgccctgaataaaagaggaaaagccaaacgagggtgcagcccccgggttaaaccccagcatatctctacccattttcttccaagattcaagcagtcggagcagccagaactttctttcacggttactgttcctgaaaagaaaaatccacctagccctatcaagtcaaagattcccctttctgcacctcggaaaaataccaactcagtgaaatacagactcaagtttcgctttggataa

SEQ ID NO: 6 - protein, 176 aa, Homo sapiens
CRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKNPPSPIKSKIPLSAPRKNTNSVKYRLKFRFG

SEQ ID NO: 7 - DNA, 531 bp, Homo sapiens
  CDS (1)..(531)
tgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataa

SEQ ID NO: 8 - protein, 176 aa, Homo sapiens
CRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 9 - DNA, 741 bp, Homo sapiens
  CDS (1)..(741)
gagaagcgtctcgcccccaaagggcaacccggacccgctgccactgataggaaccctataggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgttgccctgaataaaagaggaaaagccaaacgagggtgcagcccccgggttaaaccccagcatatctctacccattttcttccaagattcaagcagtcggagcagccagaactttctttcacggttactgttcctgaaaagaaaaatccacctagccctatcaagtcaaagattcccctttctgcacctcggaaaaataccaactcagtgaaatacagactcaagtttcgctttggataa

SEQ ID NO: 10 - protein, 246 aa, Homo sapiens
EKRLAPKGQPGPAATDRNPIGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKNPPSPIKSKIPLSAPRKNTNSVKYRLKFRFG

SEQ ID NO: 11 - DNA, 741 bp, Homo sapiens
  CDS (1)..(741)
gagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataa

SEQ ID NO: 12 - protein, 246 aa, Homo sapiens
EKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 13 - DNA, 920 bp, Homo sapiens
atgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctataggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgttgccctgaataaaagaggaaaagccaaacgagggtgcagcccccgggttaaaccccagcatatctctacccattttcttccaagattcaagcagtcggagcagccagaactttctttcacggttactgttcctgaaaagaaaaatccacctagccctatcaagtcaaagattcccctttctgcacctcggaaaaataccaactcagtgaaatacagactcaagtttcgctttggataa

SEQ ID NO: 14 - DNA, 920 bp, Homo sapiens
atgtccacccggtgcggcgaggcgggcagcgccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataa

SEQ ID NO: 15 - DNA, 1653 bp, Homo sapiens
  CDS (50)..(166)
cggacgcgtgggctctctcttcccctctccccttctcttccccgaggctatgtccacccggtgcggcgaggcgggcagcgccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataatattcctcttggccttgtgagaaaccattctttcccctcaggagtttctataggtgtcttcagagttctgaagaaaaattactggacacagcttcagctatacttacactgtattgaagtcacgtcatttgtttcagtgtgactgaaacaaaatgttttttgataggaaggaaactggaattctttgtactaatacagggagcacactccttcagttcagcaagacataaagccttttgctttatgcttgagggatatttagaactttgtattttcggaaagttaaataacagggactacgtatttttctgacttttacagattaacctgaaagaacatacatgatacatttttatttttggtttccaaagaatattttgatgcagataaaatattttgttaacttttgtttttttttgtttgttttcttaaaagtacctctgcattgagcatattttcttacttttattattttaattaatatgacataagcaatcattttatgctgtttatgaattataaatgtgtttatagctcatttgtaatatggaaatcttttacatttttcctattcactgcacttttttattgtttttatttctagccatacctcagataatatgtttagttttacattttaaaatgtttaaattctctttcacagcaaaaaaaaaaaaaaaaaaa

SEQ ID NO: 16 - protein, 38 aa, Homo sapiens
MSTRCGEAGSARGTQPHRGYRAQNQPYKMHLGPPRLEE

SEQ ID NO: 17 - DNA, 1653 bp, Homo sapiens; identical to SEQ ID NO: 15
  CDS (163)..(969)

SEQ ID NO: 18 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 19 - protein, 60 aa, Homo sapiens
KFRERFQENSYNTYASAIHRTEKTGREWYVALNKRGKAKRGCSPRVKPQHISTHFLPRFK

SEQ ID NO: 20 - DNA, 22 bp, Artificial Sequence
  Description of Artificial Sequence  Forward Primer
cttcttcagccacctgatcctc

SEQ ID NO: 21 - DNA, 21 bp, Artificial Sequence
  Description of Artificial Sequence Forward Primer
tgcagagtgggcatcggtttc

SEQ ID NO: 22 - DNA, 20 bp, Artificial Sequence
  Description of Artificial Sequence Reverse Primer
tatgctcaatgcagaggtac

SEQ ID NO: 23 - DNA, 20 bp, Artificial Sequence
  Description of Artificial Sequence  Reverse Primer
cgtagtccctgttatttaac

SEQ ID NO: 24 - DNA, 31 bp, Artificial Sequence
  Description of Artificial Sequence primer that can be used to clone HLA-A3
ttggggagggagcacaggtcagcgtgggaag

SEQ ID NO: 25 - DNA, 27 bp, Artificial Sequence
  Description of Artificial Sequence Primer that can be used to clone HLA-A3.
ggactcagaatctccccagacgccgag

SEQ ID NO: 26 - protein, 9 aa, homo sapiens
NTYASPRFK

SEQ ID NO: 27 - protein, 10 aa, Artificial Sequence
  Artificial peptide sequence.
NTYASLPRFK

SEQ ID NO: 28 - protein, 9 aa, Artificial Sequence
  Variant FGF-5 HLA-A3 epitope.
NAYASPRFK

SEQ ID NO: 29 - protein, 9 aa, Artificial Sequence
  Variant FGF-5 HLA-A3 epitope.
ATYASPRFK

SEQ ID NO: 30 - protein, 9 aa, Artificial Sequence
  Variant FGF-5 HLA-A3 epitope.
NTYAAPRFK

SEQ ID NO: 31 - protein, 9 aa, Artificial Sequence
  Variant FGF-5 HLA-A3 epitope.
NTYASARFK

SEQ ID NO: 32 - protein, 10 aa, homo sapiens
MLSVLEIFAV

SEQ ID NO: 33 - protein, 10 aa, Artificial Sequence
  Variant peptide sequence.
MFSVLEIFAV

SEQ ID NO: 34 - protein, 6 aa, homo sapiens
YASPRF

SEQ ID NO: 35 - protein, 9 aa, Artificial Sequence
  Variant peptide sequence.
NTYFLPRFK

SEQ ID NO: 36 - protein, 48 aa, Artificial Sequence
  Variant peptide sequence.
TYASAIHRTEKTGREWYVALNKRGKAKRGCSPRVKPQHISTHFLPRFK

SEQ ID NO: 37 - protein, 52 aa, Artificial Sequence
  Variant peptide sequence.
KFRERFQENSYNTYASAIHRTEKTGREWYVALNKRGKAKRGCSPRVKPQHIS

SEQ ID NO: 38 - protein, 49 aa, Artificial Sequence
  Variant peptide sequence.
NTYASAIHRTEKTGREWYVALNKRGKAKRGCSPRVKPQHISTHFLPRFK

SEQ ID NO: 39 - protein, 6 aa, Artificial
  Artificial peptide.
YAXXRF

SEQ ID NO: 40 - DNA, 21 bp, Artificial Sequence
  Description of Artificial Sequence Forward primer to PCR beta-actin.
attggcaatgagcggttccgc

SEQ ID NO: 41 - DNA, 21 bp, Artificial Sequence
  Description of Artificial Sequence reverse primer to PCR beta-actin.
aggtagtttcgtggatgccac

SEQ ID NO: 42 - DNA, 20 bp, Artificial Sequence
  Description of Artificial Sequence Forward primer to PCR FGF-5.
agtcaatggatcccacgaag

SEQ ID NO: 43 - DNA, 20 bp, Artificial Sequence
  Description of Artificial Sequence Reverse primer to PCR FGF-5.
cttgaaaacgctccctgaac

(19) United States
(12) Patent Application Publication
(10) Pub. No.: US 2006/0009393 A1
(43) Pub. Date: Jan. 12, 2006

(54) Immunogenic epitopes for fibroblast growth factors 5 (FGF-5)

(75) Inventors: Ken-Ichi Hanada, Rockville, MD (US); James C. Yang, Silver Spring, MD (US); Donna Perry-Lalley, Silver Spring, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Government of the U.S.A as represented by the Secretary of the Dept. of Health & Human Services

(21) Appl. No.: 11/134,703

(22) Filed: May 19, 2005

Related U.S. Application Data
(63) Continuation-in-part of application No. PCT/US03/37065, filed on Nov. 19, 2003.
(63) Continuation-in-part of application No. 10/089,485, filed on Mar. 27, 2002.
(60) Provisional application No. 60/427,920, filed on Nov. 19, 2002.
(60) Provisional application No. 60/157,103, filed on Oct. 2, 1999.

(51) Int. Cl. A61K 38/08; C07K 7/06

(52) U.S. Cl. 514/17; 530/328

(57) ABSTRACT

The present disclosure relates to peptides for use in immunotherapy of FGF-5 expressing tumors. In one example, the peptide is an HLA-A3 epitope (such as NTYASPRFK). In another example, the peptide is an HLA-A2 epitope (such as MLSVLEIFAV). Methods are provided for using such peptides (and corresponding nucleic acid molecules), and variants, fragments or fusions thereof, to stimulate an immune response in a subject. The peptides (and corresponding nucleic acid molecules) disclosed herein can be formulated into pharmaceutical composition for administration to a subject.

### CROSS REFERENCE TO RELATED APPLICATIONS

This application is a continuation-in-part of PCT/US03/37065, filed Nov. 19, 2003, which claims the benefit of U.S. Provisional Application No. 60/427,920 filed Nov. 19, 2002, and is a continuation-in-part of U.S. application Ser. No. 10/089,485 filed Mar. 27, 2002, which is a U.S. national stage of PCT/US00/26689 filed Sep. 29, 2000, which claims the benefit of U.S. Provisional Application No. 60/157,103 filed Oct. 2, 1999, all herein incorporated by reference in their entirety.

### FIELD

This application relates to human leukocyte antigen (HLA) epitopes of fibroblast growth factor 5 (FGF-5) and uses thereof, for example to stimulate a cytotoxic T cell response against a neoplasm (such as a tumor) expressing or overexpressing FGF-5.

### BACKGROUND

The use of immunologic agents has been proposed as an alternative for anti-neoplastic chemotherapy. The process by which T cells recognize and interact with other cells involves cell surface complexes on the other cells of peptides and molecules referred to as human leukocyte antigens (HLA) or major histocompatibility complexes (MHC). The interaction of T cells and complexes of MHC with an antigen is restricted, requiring a specific T cell for a specific complex of MHC and peptide antigen. For example, particular antigens have been identified which are expressed on cell surfaces, which can lead to lysis of the tumor cells by specific cytotoxic T cells. Genes that encode the antigens found on the surface of the cancer are called tumor rejection antigen precursors (TRAP) molecules, and the peptides derived from these genes are referred to as tumor rejection antigens (TRA) or tumor associated antigens (TAA). However, only a few TAA molecules have been recognized, and these TAAs are only known to be present on a limited number of tumor types (for example see U.S. Pat. No. 5,939,526).

Beginning with an initial description of an antigen on melanoma recognized by T-cells from a patient repetitively vaccinated with autologous tumor (van der Bruggen et al., 1991. Science 254:1643-7), dozens of other melanoma-associated antigens have been identified which provoke T-cell responses (Boon et al., 1997. Immunol. Today 18:267-8; Robbins et al., 2000. Tumor Antigens Recognized by Cytotoxic Lymphocytes. In Cytotoxic Cells: Basic Mechanisms and Medical Applications. Sitkovsky and Henkart, editors. J. B. Lippincott, Philadelphia. 363-383; Van den Eynde and B. P. van der. 1997. Curr. Opin. Immunol. 9:684-93).

These results have led to efforts to identify similar T-cells and tumor-associated antigens for IL-2 responsive tumors other than melanoma. However, this search has had limited success. For example, although several CTL clones directed against renal cell carcinoma (RCC) have been obtained, many of the known RCC antigens are expressed in normal tissue, or are infrequently found when testing large numbers of individual renal cancers, making them less than ideal for tumor therapy. Other antigens appear to be poorly processed and presented by actual tumor cells (as opposed to target cells incubated with the [already processed] minimal peptide epitopes).

Each year in the United States, approximately 30,000 patients are diagnosed with renal cell carcinoma (RCC) and approximately 12,000 patients die of this disease (Linehan et al., 2001. Cancers of the Genitourinary System. In Cancer: Principles and Practice of Oncology. DeVita, Hellman, and Rosenberg, editors. Lippincott Williams and Wilkins, Philadelphia. 1343-1489). Most patients initially present with either advanced local disease or metastatic disease. Metastatic disease has a dismal prognosis and even patients with advanced local disease are likely to develop metastases and die, despite successful nephrectomy.

Chemotherapy is largely ineffective for unresectable metastatic disease. Instead, the only U.S. Food and Drug Administration (FDA) approved therapy for metastatic disease is immunotherapy with high-dose bolus interleukin 2 (IL-2). Although IL-2 can cause regression in about 15-20% of patients, only about one third of these are complete responses. If patients attain a complete response to high-dose IL-2, their chances of being alive and free of disease after a median follow-up of over nine years is 80% (Rosenberg et al., 1998. Ann. Surg. 228:319; Fisher et al., 1997. Cancer J Sci. Amer. 3:S70-S72). Therefore, new methods are needed to improve on IL-2 therapy and expand its curative potential to a greater percentage of patients with RCC.

Therefore, there is a need to identify RCC antigens that contain naturally-processed peptide epitopes, and which are highly expressed on a significant proportion of RCC tumors but with little or no expression on normal adult tissues. In addition, there is a need to identify tumor antigens which extend effective cancer immunotherapy beyond just melanoma and RCC, for example to common adenocarcinomas (such as breast, prostate, and pancreatic cancer) which constitute a large portion of all human cancers and for which there are few or no successful immunotherapy approaches.

### SUMMARY

Disclosed herein are novel HLA-A3 and HLA-A2 FGF-5 epitopes, which can stimulate an immune response, and in some examples, can be used to treat an FGF-5 expressing (or overexpressing) neoplasm (such as a tumor). HLA-A molecules are Class I MHC antigens, which serve as target antigens for immune recognition and killing. HLA-A2 and HLA-A3 molecules are alternate forms of HLA-A molecules that recognize distinct epitopes of a single protein, such as FGF-5.

Although particular FGF-5 HLA-A3 epitopes are disclosed herein, the disclosure is not limited to these particular examples. For example, the disclosure provides purified, immunogenic peptides which include the peptide sequence Tyr-Ala-(A³)-(A⁴)-Arg-Phe, wherein A³ is Ala or Ser and A⁴ is Ala or Pro (SEQ ID NO: 39), as well as variants, fragments, and fusions thereof that can stimulate an immune response, such as peptides that include SEQ ID NO: 34 (amino acids 3-9 of SEQ ID NO: 26). In a particular example, such peptides are at least eight amino acids, or no more than 12 amino acids, and in particular examples are 8-12 amino acids or even 8-10 amino acids in length. In one example, such variants are recognized by, or can generate an immune cell (nominally a T-cell) that specifically reacts with a sequence that includes SEQ ID NO: 34. Other particular examples of FGF-5 HLA-A3 epitopes include, but are not limited to, a sequence which includes the peptide sequence shown in any of SEQ ID NOS: 26-31, as well as variants, fragments, and fusions thereof that can stimulate an immune response.

Although particular FGF-5 HLA-A2 epitopes are disclosed herein, the disclosure is not limited to these particular examples. For example, the disclosure provides purified, immunogenic peptides which include the peptide sequence shown in SEQ ID NO: 32, as well as variants, fragments, and fusions thereof that can stimulate an immune response. In one example, such variants are recognized by, or can promote production of an immune cell (nominally a T cell), that specifically reacts with SEQ ID NO: 32.

Nucleic acid molecules encoding the disclosed FGF-5 HLA-A2 and -A3 peptides (as well as the disclosed variants, fragments, and fusions thereof) are also encompassed by this disclosure, as well as vectors and host cells that include such nucleic acid molecules.

Also provided herein are pharmaceutical compositions that include the FGF-5 HLA-A2 or -A3 epitope (or nucleic acids encoding such peptides). Such compositions can include a pharmaceutically effective carrier (such as an adjuvant), or other therapeutic agents (such as an antineoplastic agent).

Methods are also disclosed for eliciting or enhancing an immune response in a subject, using the disclosed HLA-A3 and HLA-A2 FGF-5 epitopes, or a nucleic acid molecule encoding such peptides, or immunoreactive T cells sensitized with an FGF-5 HLA-A2 or -A3 epitope. In particular examples, such methods can be used to treat a subject having an FGF-5 expressing or overexpressing tumor or neoplasm (such as an adenocarcinoma, for example RCC), for example by stimulating a cytotoxic T cell response to cells of the tumor, thereby treating the subject. In particular examples the method includes administering a therapeutically effective amount of an FGF-5 HLA-A2 or HLA-A3 epitope (or a nucleic acid molecule encoding such peptides), alone, or in the presence of other therapeutically effective molecules, such as IL-2 or immunoreactive sensitized T cells sensitized with an FGF-5 HLA-A2 or -A3 epitope. In some examples, whether the tumor present in the subject expresses or overexpresses FGF-5 is determined, wherein subject's having an FGF-5 expressing or overexpressing tumor can receive the disclosed therapy.

Administration of the disclosed FGF-5 HLA-A2 and HLA-A3 epitoptic peptides (or nucleic acid molecules encoding them or immunoreactive T cells sensitized with the peptides) can be used to induce an immune response in a subject against these tumor antigens. In one example, the HLA haplotype of the subject is determined prior to administration of the disclosed peptides (or nucleic acid molecule). For example, individuals can be pre-screened to select HLA-A3+ or A2+ individuals to whom to administer an FGF-5 HLA-A3 or -A2 epitope. For example, if the individual is HLA-A3+, that individual can be administered one or more FGF-5 HLA-A3 epitopes, and if the individual is HLA-A2+, that individual can be administered one or more FGF-5 HLA-A2 epitopes.

Methods of producing antibodies specific for an FGF-5 antigen are also disclosed, for example by introducing the disclosed FGF-5 HLA-A2 and HLA-A3 antigens (or variants thereof) into a subject, and allowing the subject to generate antibodies that recognize such antigens, or the variants thereof. In particular examples, such methods can be used to treat a subject having an FGF-5 expressing or overexpressing tumor or neoplasm.

The foregoing and other objects, features, and advantages of the disclosure will become more apparent from the following detailed description, which proceeds with reference to the accompanying figures.

### BRIEF DESCRIPTION OF THE FIGURES

FIGS. 1A and 1B are bar graphs showing the effect of various anti-HLA monoclonal antibodies on the recognition of tumors by (A) an HLA-A2 restricted CTL or (B) Clone 2 CTL.

FIG. 2 is a schematic drawing showing the alignment of the three tumor-derived FGF-5 clones. The nucleotide changes (inside each bar), their positions (under 10E4-1), and any resulting amino acid changes (above each bar) are shown.

FIG. 3 is a graphical representation showing FGF-5 peptides that are immunogenic (unfilled bars) and are non-immunogenic (filled bars). The numbers below each bar represent the nucleotide number (upper numbers), and amino acid number (lower numbers) of the FGF-5 sequences shown in SEQ ID NO: 39.

FIG. 4 shows the sequences of the FGF-5 internal deletion mutants. All constructs were designed to start with the Kozak consensus sequence and end with the termination codon TAA. Numbers in brackets stand for the position of the first and the last amino acids. Numbers following A indicate internally deleted amino acid residues. The degree of CTL recognition is categorized as follows depending on IFN-γ secretion: (++)>1000 pg/ml, (+) 200-1000 pg/ml, (+/−) 100-200 pg/ml, (−)<100 pg/ml.

FIG. 5 is bar graph showing the results of alanine substitutions on an HLA-A3 epitope sequence.

FIG. 6 is a bar graph showing FGF-5 peptides that generate a significant IFN-γ response.

FIG. 7 is a graph showing the titration of three FGF-5 peptides. The fusion peptides (NTYASPRFK, NTYASLPRFK, NTYFLPRFK; SEQ ID NOS: 26, 27 and 35, respectively) were pulsed onto the autologous EBV-B cell line at the concentrations indicated and their recognition by C2 was assessed by IFN-γ assay. Error bars represent the standard deviation of duplicate IFN-γ determinations.

FIG. 8A is a schematic representation of extended FGF-5 synthetic peptides. FGF-5 (173-220; SEQ ID NO: 36). The 48-mer peptide lacks the first N-terminal asparagine of NTYAS but includes C-terminal PRFK; FGF-5 (161-212; SEQ ID NO: 37). The 52-mer peptide includes NTYAS but lacks PRFK; FGF-5 (172-220 SEQ ID NO: 38). The 49-mer peptide starts with NTYAS and ends with PRFK; FGF-5 (161-220; SEQ ID NO: 39).

FIGS. 8B and 8C are bar graphs showing the recognition of the peptides in FIG. 8A by a control RCC-reactive CTL from the same patient not recognizing FGF-5 (B) or FGF-5-reactive Clone 2 CTL (C). Functional specificity of each CTL line is shown in each side bar using the autologous EBV-B cell line and the autologous RCC cell line as targets.

FIG. 9 is a graph showing that fresh and fixed EBV-B cells have a similar capacity to present the 9-mer determinant NTYASPRFK (SEQ ID NO: 26). Error bars represent the standard deviation of duplicate IFN-γ determination.

FIGS. 10A-10D are graphs showing the ability of HPLC fractionated (A) synthetic 9-mer (SEQ ID NO: 26), (B) synthetic 49-mer (SEQ ID NO: 38), and acid-stripped peptides from (C)COS-A3 or (D) COS-A3/FGF-5 to be recognized by Clone 2. Error bars represent the standard deviation of duplicate IFN-γ determination.

FIGS. 11A and 11B are bar graphs showing that presentation of both MHC class I-restricted peptides was blocked by clasto-Lactacystin \tilde{β}-lactone or by expressing ICP47 to inhibit TAP-mediated cytosol to ER peptide transport. (A) Open bars: untreated cells, filled bars: cells incubated with clasto-Lactacystin β-lactone. (b) RCC cell line was infected with either an adenovirus encoding GFP (open bars) or TAP-1 inhibitor ICP47 (filled bars). Bars represent the average IFN-γ secretion and the error bars, the standard deviation of duplicate samples. Similar results were repeated and representative results are shown.

FIG. 12 is a bar graph showing CTL clones that recognized an FGF-5 HLA-A2 epitope.

FIG. 13 is a bar graph showing the results of a L118F amino acid substitution (A516C nucleotide substitution) in an FGF-5 HLA-A2 epitope.

### SEQUENCE LISTING

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NOS: 1 and 2 shows a DNA and an amino acid sequence, respectively, for a first open reading frame of MUSFGF-5A.

SEQ ID NOS: 3 and 4 shows a DNA and an amino acid sequence, respectively, for a second open reading frame of MUSFGF-5A.

SEQ ID NOS: 5 and 7 show cDNA sequences for variations of IA3-1.

SEQ ID NOS: 6 and 8 show amino acid sequences for variations of IA3-1.

SEQ ID NOS: 9 and 11 show cDNA sequences for variations of 6A4-1.

SEQ ID NOS: 10 and 12 show amino acid sequences for variations of 6A4-1.

SEQ ID NO: 13 shows a cDNA sequence for a variation of 10E4-1.

SEQ ID NO: 14 shows a cDNA sequence for another variation of 10E4-1.

SEQ ID NOS: 15 and 16 show a full-length cDNA sequence, and the corresponding protein sequence, respectively, of construct 10E4-1 with ORF-1.

SEQ ID NOS: 17 and 18 show a full-length cDNA sequence, and the corresponding protein sequence, respectively, of construct 10E4-1 with ORF-2.

SEQ ID NO: 19 shows an amino acid sequence which includes an FGF-5 HLA-A3 epitope.

SEQ ID NOS: 20 and 21 show nucleic acid sequences of forward primers that can be used to RT-PCR FGF-5.

SEQ ID NOS: 22 and 23 show nucleic acid sequences of reverse primers that can be used to RT-PCR FGF-5.

SEQ ID NOS: 24 and 25 show nucleic acid sequences of forward and reverse primers, respectively, that can be used to clone the HLA-A3 gene from autologous 1764 RCC by RT-PCR.

SEQ ID NO: 26 shows an amino acid sequence of a 9-mer FGF-5 HLA-A3 epitope.

SEQ ID NO: 27 shows an amino acid sequence of a 10-mer FGF-5 HLA-A3 epitope.

SEQ ID NOS: 28-31 show amino acid sequences, which are variants of SEQ ID NO: 26 and function as an FGF-5 HLA-A3 epitope.

SEQ ID NO: 32 shows an amino acid sequence of a 10-mer FGF-5 HLA-A2 epitope.

SEQ ID NO: 33 shows an amino acid sequence variant of SEQ ID NO: 32 (L118F), which does not function as an FGF-5 HLA-A2 epitope.

SEQ ID NO: 34 shows amino acids 3 to 8 (YASPRF) of SEQ ID NO: 26; that is amino acids 174-176 and 217-219 of FGF-5.

SEQ ID NO: 35 shows a variant amino acid sequence of SEQ ID NO: 26, which does not function as an FGF-5 HLA-A3 epitope.

SEQ ID NO: 36 shows amino acids 173-220 of FGF-5.

SEQ ID NO: 37 shows amino acids 161-212 of FGF-5.

SEQ ID NO: 38 shows amino acids 172-220 of FGF-5.

SEQ ID NO: 39 shows a variant FGF-5 HLA-A3 epitope.

SEQ ID NOS: 40 and 41 show forward primers and reverse primers, respectively, that can be used to RT-PCR B-actin.

SEQ ID NOS: 42 and 43 show forward primers and reverse primers, respectively, that can be used to RT-PCR FGF-5.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein, the singular forms “a” or “an” or “the” include plural references unless the context clearly dictates otherwise. For example, reference to “an FGF-5 HLA-A2 epitope” includes one or a plurality of such epitopes and reference to “the tumor” includes reference to one or more tumors and equivalents thereof known to those skilled in the art, and so forth. The term “or” refers to a single element of stated alternative elements or a combination of two or more elements, unless the context clearly indicates otherwise. For example, the phrase “an FGF-5 HLA-A2 epitope or an FGF-5 HLA-A3 epitope” refers to an FGF-5 HLA-A2 epitope, an FGF-5 HLA-A3 epitope, or a combination of both an FGF-5 HLA-A2 epitope and an FGF-5 HLA-A3 epitope. As used herein, “comprises” means “includes.” Thus, “comprising an FGF-5 HLA-A3 epitope and IL-2,” means “including an FGF-5 HLA-A3 epitope and IL-2,” without excluding additional elements.

Unless defined otherwise, all technical and scientific terms used herein have the same meaning as commonly understood to one of ordinary skill in the art to which this disclosure belongs.

| | |
|---|---|
| CTL | cytotoxic T lymphocyte |
| FGF-5 | fibroblast growth factor-5 |
| HLA | human leukocyte antigen |
| RCC | renal cell carcinoma |
| RT | room temperature |
| SDS-PAGE | sodium dodecyl sulfate-polyacrylamide gel electrophoresis |
| TAA | tumor-associated antigen |
| TCR | T-cell receptor |
| TILs | tumor infiltrating lymphocytes |

Adjuvant: An agent that when used in combination with an immunogenic agent augments or otherwise alters or modifies a resultant immune response. In some examples, an adjuvant increases the titer of antibodies induced in a subject by the immunogenic agent, or increases a T cell response in a subject by the immunogenic agent (such as an increase in the number of CD4⁺ or CD8⁺ cells).

Exemplary adjuvants include, but are not limited to, Freund's Incomplete Adjuvant (IFA), Freund's complete adjuvant, ISA-51 (Seppic, Inc.), QS-21 (Antigenics; Aquila Biopharmaceuticals, Framingham, Mass.), MPL (SmithKline Beecham), B30-MDP, LA-15-PH, montanide, saponin, aluminum salts such as aluminum hydroxide (Amphogel, Wyeth Laboratories, Madison, N.J.), alum, lipids, keyhole lympet protein, hemocyanin, edestin, the MF59 microemulsion, a mycobacterial antigen, vitamin E, non-ionic block polymers, muramyl dipeptides, polyanions, amphipatic substances, ISCOMs (immune stimulating complexes, such as those disclosed in European Patent EP 109942), vegetable oil, Carbopol, aluminium oxide, oil-emulsions (such as Bayol F or Marcol 52), bacterial toxins (such as B. anthracis protective antigen, E. coli heat-labile toxin (LT), Cholera toxin, tetanus toxin/toxoid, diphtheria toxin/toxoid, P. aeruginosa exotoxin/toxoid/, pertussis toxin/toxoid, and C. perfringens exotoxin/toxoid), bacterial wall proteins and other products (such as cell walls and lipopolysaccharide (LPS)) and combinations thereof.

Agent: Any substance, including, but not limited to, an antibody, chemical compound, molecule, peptidomimetic, or protein.

Antigen: A compound, composition, or substance that can stimulate the production of antibodies or a T-cell response in a subject, including compositions that are administered to a subject. An antigen reacts with the products of specific humoral or cellular immunity, including those induced by heterologous immunogens. The term “antigen” includes all related antigenic epitopes.

Antineoplastic agent: A drug or biologic that decreases or in some examples inhibits the proliferation of neoplastic cells. In one example, administration of an antineoplasitic agent to neoplastic cells arrests their growth or causes regression of a tumor.

Examples include alkylating agents (such a vincristine, vinblastine or taxol), anthracycline antibiotics such as daunorubicin and doxorubicin, hormonal therapies such as tamoxifen, and agents such as cis-diamminedichloroplatimun (II) and hydroxyurea. Antineoplastic agents also include biologics, such as IL-2 and alpha-interferon, and immunotherapy, for example with bacille Calmette-Guerin (BCG). Protocols for administration of such agents are known in the art, and examples can be found in Goodman and Gilman, The Pharmacological Basis of Therapeutics, 17^{th} edition, section XIII.

Cancer: Malignant neoplasm that has undergone characteristic anaplasia with loss of differentiation, increased rate of growth, invasion of surrounding tissue, and is capable of metastasis.

cDNA (complementary DNA): A piece of DNA lacking internal, non-coding segments (introns) and regulatory sequences which determine transcription. cDNA can be synthesized in the laboratory, for example by reverse transcription from messenger RNA extracted from cells.

Conservative substitution: One or more amino acid substitutions for amino acid residues having similar biochemical properties. Typically, conservative substitutions have little to no impact on the activity of a resulting polypeptide. For example, a conservative substitution is an amino acid substitution in an antigenic epitope of an FGF-5 peptide that does not substantially affect the ability of an FGF-5 reactive T-cell to recognize the peptide. In a particular example, a conservative substitution is an amino acid substitution in an antigenic epitope of an FGF-5 peptide, such as a conservative substitution in any of SEQ ID NOS: 26-31, 34 and 39, which does not significantly decrease recognition of the epitope by Clone 2 or significantly decrease recognition of the epitope by an HLA-A2 positive tumor cell (for HLA-A2 FGF-5 epitopes, such as SEQ ID NO: 32).

Methods that can be used to determine the amount of recognition by a variant epitope are disclosed herein (for example, see Examples 5-7 and 11). For example, an alanine scan can be used to identify which amino acid residues in an HLA-A2 or -A3 FGF-5 epitope can tolerate an amino acid substitution (for example see Example 6). In one example, recognition is not decreased by more than 25%, for example not more than 20%, for example not more than 10%, when an alanine, or other conservative amino acid (such as those listed below), is substituted for one or more native amino acids.

In one example, one conservative substitution is included in the peptide, such as a single conservative amino acid substitution in any of SEQ ID NOS: 26-32, 34 OR 39. In another example, two conservative substitutions are included in the peptide. In a further example, three conservative substitutions are included in the peptide. A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods.

Substitutional variants are those in which at least one residue in the amino acid sequence has been removed and a different residue inserted in its place. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Further information about conservative substitutions can be found in, among other locations in, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Deletion: The removal of one or more nucleotides from a nucleic acid sequence, or the removal of one or more amino acids from a protein sequence, the regions on either side being joined together.

Degenerate variant: A nucleic acid sequence that encodes a peptide, such as an FGF-5 HLA-A2 or -A3 epitope, that includes a sequence that is degenerate as a result of the genetic code. There are 20 natural amino acids, most of which are specified by more than one codon. Therefore, multiple degenerate nucleotide sequences can encode the same peptide sequence.

Enhance: To improve the quality, amount, or strength of something. In one example, a therapy enhances the immune system if the immune system is more effective at reducing tumors than in the absence of the therapy. In a particular example, an FGF-5 HLA-A2 or HLA-A3 epitope, or combinations thereof, enhances the benefit of IL-2 therapy in a subject having one or more tumors. Such enhancement can be measured using any bioassay known in the art, for example, by measuring an amount of IFN-γ production by an ELISA assay or microscopy (for example see Example 11).

In one example, an FGF-5 HLA-A2 or HLA-A3 epitope, or combinations thereof, enhances an immune response as measured by clinical response. One example of a clinical response is an increase in a population of immune cells, such as an increase of at least 10%, for example at least 20 or even at least 50%, compared to the number of immune cells in the absence of the therapy. Another example of a clinical response is a measurable reduction in the size of a tumor, for example a reduction in size of at least 5%, such as at least 10%, at least 20, or even at least 50%.

Epitope: An antigenic determinant, such as chemical groups or peptide sequences that elicit a specific immune response. An antibody binds a particular antigenic epitope, or a T-cell reacts with a particular antigenic epitope bound to a specific MHC molecule.

For example, FGF-5 HLA-A2 and HLA-A3 epitopes are peptide sequences that recognize distinct regions of FGF-5, but both can elicit an immune response against FGF-5. Exemplary FGF-5 HLA-A2 epitopes include, but are not limited to, SEQ ID NO: 32 as well as variants, fragments, and fusions thereof that retain the ability to stimulate an immune response against FGF-5. Exemplary FGF-5 HLA-A3 epitopes include, but are not limited to, SEQ ID NOS: 26-31, 34 and 39, as well as variants, fragments, and fusions thereof that retain the ability to stimulate an immune response against FGF-5.

Fibroblast growth factor 5 (FGF-5): FGF-5 (initially termed FGF-3) is an oncogene-encoded glycoprotein bearing mitogenic activity for fibroblasts and endothelial cells. Includes both naturally occurring and recombinant FGF-5 cDNA, RNA, or protein from any organism, as well as FGF-5 fragments and FGF-5 variants that retain full or partial FGF-5 biological activity. Exemplary FGF-5 nucleic acid sequences include Genbank Accession Nos: M37825 and NM_{—}004464 (human) and NM_{—}010203 (mouse) and exemplary amino acid sequences include Genbank Accession Nos: AAB06463 and NP_{—}004455 (human) and NP_{—}034333 (mouse). However, those skilled in the art will appreciate that other FGF-5 sequences are publicly available for several other organisms.

FGF-5 Expressing Tumor: A tumor, such as a neoplasm, which expresses or over-expresses wild-type or mutant FGF-5. In a particular example, such FGF-5 expression is relative to an amount of FGF-5 expression in a normal or non-tumor cell, for example a non-tumor cell of the same type as the tumor. For example, an FGF-5 expressing tumor can be one in which there is increased FGF-5 expression or copy number, relative to FGF-5 expression in a same tissue type that is non-neoplastic. In a specific example, FGF-5 expression or overexpression is determined by RT-PCR, for example as described in Example 15.

Examples of such tumors include, but are not limited to: a carcinoma, for example an adenocarcinoma, such as cancers of the breast, kidney (renal cell carcinoma), prostate, bladder, and pancreas.

FGF-5 HLA-A2 or -A3 fusion protein: A protein that includes an FGF-5 HLA-A2 or -A3 epitope sequence linked to one or more other amino acids that do not significantly decrease the immunogenic activity of the epitope sequence. Such amino acids can be linked to the N- or C-terminus of the epitope sequence. The other amino acid sequences can be, for example, no more than 10, 20, 30, or 50 amino acid residues in length.

Functionally equivalent peptide: The ability of a peptide containing one or more sequence alterations to retain a function of the unaltered peptide. Examples of sequence alterations include, but are not limited to, conservative substitutions, deletions, mutations, frameshifts, insertions, and combinations thereof.

In a particular example, an FGF-5 eptiope including one or more sequence alterations retains a function of the unaltered epitope. In one example, the variant FGF-5 eptiope specifically binds an antibody that also binds to an unaltered form of an FGF-5 eptiope. In another or additional example, the variant FGF-5 eptiope retains the ability to be recognized by Clone 2 (for HLA-A3 FGF-5 epitopes, such as SEQ ID NO: 26-31) or by an HLA-A2 positive tumor cell (for HLA-A2 FGF-5 epitopes, such as SEQ ID NO: 32).

In one example, a particular peptide binds an antibody, and a functional equivalent of that particular peptide is another peptide that binds the same antibody. Thus a functional equivalent includes peptides which have the same binding specificity as a polypeptide, and which may be used as a reagent in place of the peptide (such as in a diagnostic assay or vaccine). In a particular example, a functional equivalent includes a polypeptide having a discontinuous binding sequence, and the antibody binds a linear epitope. Thus, if the peptide sequence is MLSVLEIFAV (SEQ ID NO: 32) a functional equivalent includes discontinuous epitopes, which may can appear as follows (**=any number of intervening amino acids):

NH2-**-M**L**S**V**L**E**I**F**A**V-COOH. This polypeptide is functionally equivalent to SEQ ID NO: 7 if the three dimensional structure of the polypeptide is such that it can bind a monoclonal antibody that binds SEQ ID NO: 32, or if it retains the ability to be recognized by an HLA-A2 positive tumor cell (see Example 7).

Haplotype: A set of alleles of a group of closely linked genes, such as the human leukocyte antigen (HLA) complex, which are usually inherited as a unit, an individual inheriting a complete haplotype from each parent. In one example, it is the genetic constitution of an individual at a set of linked genes.

Haplotyping or tissue typing: A method used to identify the haplotype or tissue types of a subject, for example by determining which HLA locus (or loci) is expressed on the lymphocytes of a particular subject. The HLA genes are located in the major histocompatibility complex (MHC), a region on the short arm of chromosome 6, and are involved in cell-cell interaction, immune response, organ transplantation, development of cancer, and susceptibility to disease. There are five genetic loci, designated HLA-A, HLA-B, HLA-C, HLA-D, and HLA-DR. At each locus, there can be any of several different alleles.

Immune response: A change in immunity, for example, a response of a cell of the immune system, such as a B cell, T cell, or monocyte, to a stimulus (such as an immunogenic agents, for example an epitope). The response can be specific for a particular antigen (an “antigen-specific response”). In one example, an immune response is a T cell response, such as a CD4⁺ response or a CD8⁺ response. In another example, the response is a B-cell response, and results in the production of specific antibodies to the immunogenic agent. In a particular example, an increased or enhanced immune response is an increase in the ability of a subject to treat a disease, such as a tumor.

A stimulated, enhanced or elicited immune response is the enhancement of an immune response in a subject, such as a CTL immune response, for example an HLA-A3- or HLA-A2-restricted CTL response against an FGF-5 expressing or over-expressing tumor. In one example, an immune response is stimulated if following the administration of an FGF-5 HLA-A3 or HLA-A2 epitope, the amount of gamma interferon (IFN-γ) produced by a peripheral blood mononuclear cell (PBMC) of the subject to whom the epitope was administered increases by at least 10% (such as at least 50%) as compared to a response prior to administering the epitope. Examples of agents that can stimulate an immune response, include, but are not limited to: FGF-5 epitopes such as SEQ ID NOS: 26-32, 34 and 39 (and variants and fusions thereof), nucleic acid molecules encoding such peptides, and immunoreactive sensitized T cells sensitized with an FGF-5 epitope.

Immune stimulatory composition: A pharmaceutical composition which includes an antigen (such as one or more FGF-5 HLA-A2 epitope antigens, HLA-A3 epitope antigens, or combinations thereof), which when administered to a subject, results in the subject producing antibodies against the antigen, producing cytotoxic T cells that recognize the antigen, or combinations thereof. In particular examples, the subject's response to the composition results in treatment of the subject having an FGF-5 expressing or over-expressing tumor.

Isolated: An “isolated” biological component (such as a nucleic acid molecule or protein) has been substantially separated, produced apart from, or purified away from other biological components in the cell of the organism in which the component naturally occurs, such as other chromosomal and extrachromosomal DNA and RNA, and proteins. Nucleic acid molecules and proteins which have been “isolated” include nucleic acid molecules and proteins purified by standard purification methods. The term also embraces nucleic acids and proteins prepared by recombinant expression in a host cell as well as chemically synthesized nucleic acid molecules and proteins.

Leukocyte: Cells in the blood, also termed “white cells,” that are involved in defending a subject against infective organisms and foreign substances. Leukocytes are produced in the bone marrow. There are 5 main types, subdivided between 2 main groups: polymorphonuclear leukocytes (neutrophils, eosinophils, basophils) and mononuclear leukocytes (monocytes and lymphocytes).

Lymphocytes: A type of white blood cell that is involved in the immune defenses of the body. There are two main types of lymphocytes: B-cell and T-cells.

Malignant: Cells which have the properties of anaplasia invasion and metastasis.

Neoplasm: Abnormal growth of cells.

Normal cells: Non-tumor, non-malignant cells.

Nucleic acid molecule: A deoxyribonucleotide or ribonucleotide polymer in either single or double stranded form, and unless otherwise limited, encompasses known analogues of natural nucleotides that hybridize to nucleic acids in a manner similar to naturally occurring nucleotides.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Peptide: A chain of amino acids that is at least 4 amino acids in length, regardless of post-translational modification (such as glycosylation or phosphorylation). In one example, a peptide is at least 6 amino acids in length, such as at least 8, at least 9, at least 10, at least 11, or at least 12 amino acids in length. In particular examples, a peptide is about 4 to about 30 amino acids, for example 6 to 25 amino acids, 8 to 25 amino acids, 6 to 10 amino acids, 9 to 15 amino acids in length, or 9-10 amino acids in length. In one example, a peptide is an FGF-5 epitope, such as a sequence that includes any of SEQ ID NOS: 26-32, 34 and 39 (or variants, fragments, or fusions thereof).

Pharmaceutical agent or drug: A chemical compound or composition capable of inducing a desired therapeutic effect when properly administered to a subject.

Pharmaceutically acceptable carriers: The pharmaceutically acceptable carriers useful herein are conventional. Remington's Pharmaceutical Sciences, by E. W. Martin, Mack Publishing Co., Easton, Pa., 15th Edition (1975), describes compositions and formulations suitable for pharmaceutical delivery of the pharmaceutical compositions disclosed herein. Particular examples of pharmaceutically acceptable carriers include, but are not limited to physiologically acceptable fluids and adjuvants.

Probes and primers: Nucleic acid probes and primers may readily be prepared based on the sequences provided herein. A probe includes an isolated nucleic acid molecule attached to a detectable label or reporter molecule. Typical labels include radioactive isotopes, ligands, fluorophores, chemiluminescent agents, and enzymes. Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Primers are short nucleic acid molecules, such as DNA oligonucleotides 12-50 nucleotides in length. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example by PCR or other nucleic-acid amplification methods known in the art.

Methods for preparing and using probes and primers are described, for example, in Sambrook et al. (Molecular Cloning: A Laboratory Manual, Cold Spring Harbor Laboratory Press, 1989), Ausubel et al., 1987, and Innis et al., PCR Protocols, A Guide to Methods and Applications, 1990, Innis et al. (eds.), 21-27, Academic Press, Inc., San Diego, Calif. PCR primer pairs can be derived from a known sequence, for example, by using computer programs intended for that purpose such as Primer (Version 0.5, @ 1991, Whitehead Institute for Biomedical Research, Cambridge, Mass.).

Promoter: An array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Both constitutive and inducible promoters are included (Bitter et al., Meth. Enzymol. 153:516-44, 1987).

Purified: The term purified does not require absolute purity; rather, it is intended as a relative term. Thus, for example, a purified peptide preparation is one in which the protein is more enriched than the protein is in its environment within a cell, such that the peptide is substantially separated from cellular components (such as nucleic acid molecules, lipids, carbohydrates, and other peptides) that may accompany it. In another example, a purified peptide preparation is one in which the peptide is substantially-free from contaminants, such as those that might be present following chemical synthesis of the peptide.

In one example, an FGF-5 HLA-A2 or -A3 epitope peptide is purified when at least 60% by weight of a sample is composed of the peptide, for example when 75%, 95%, or 99% or more of a sample is composed of the peptide. Examples of methods that can be used to purify an antigen, include, but are not limited to the methods disclosed in Sambrook et al. (Molecular Cloning: A Laboratory Manual, Cold Spring Harbor, N.Y., 1989, Ch. 17). Protein purity can be determined by, for example, polyacrylamide gel electrophoresis of a protein sample, followed by visualization of a single polypeptide band upon staining the polyacrylamide gel; high-pressure liquid chromatography; sequencing; or other conventional methods. The methods disclosed herein can include administration of purified FGF-5 epitope protein to a subject to provoke a CTL response against a tumor that is expressing or overexpressing FGF-5.

Recombinant: A recombinant nucleic acid molecule or protein is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Sample: Includes biological samples that include cells, genomic DNA, RNA, or proteins (or combinations thereof) obtained from a subject, such as those present in peripheral blood, urine, saliva, tissue biopsy, surgical specimen, fine needle aspirate, amniocentesis samples and autopsy material.

Sequence identity: The similarity between two or more nucleic acid sequences, or two or more amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a nucleic acid or amino acid sequence possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al,. J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N₈O₅, Bethesda, Md. 20894) and on the Internet, for use in connection with the sequence analysis programs blastp, blastn, blastx, tblastn and tblastx. Additional information can be found at the NCBI web site. Alternatively, one may manually align the sequences and count the number of identical amino acids (or nucleotides). This number divided by the total number of amino acids (or nucleotides) in the reference sequence multiplied by 100 results in the percent identity.

Variants of an FGF-5 HLA-A2 or -A3 epitope are typically characterized by possession of at least 77%, at least 85%, at least 90%, at least 95%, at least 98%, or at least 99% sequence identity counted over full-length alignment with the amino acid sequence of an FGF-5 HLA-A2 or -A3 epitope (such as SEQ ID NOS: 26-32, 34 or 39) using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment is performed using the Blast 2 sequences function, employing the PAM30 matrix set to default parameters (open gap 9, extension gap 1 penalties). When less than the entire FGF-5 sequence is being compared for sequence identity, for example an FGF-5 epitope, variants typically possess at least 75% sequence identity over short windows of 6-20 amino acids, and can possess sequence identities of at least 85%, 90%, 95%, 98% or 99% depending on their similarity to the reference sequence. Methods for determining sequence identity over such short windows are described at the NCBI web site. One of skill in the art will appreciate that these sequence identity ranges are provided for guidance only; it is entirely possible that strongly significant homologs could be obtained that fall outside of the ranges provided.

Queries searched with the blastn program are filtered with DUST (Hancock, and Armstrong, 1994, Comput. Appl. Biosci. 10:67-70). Other programs use SEG. Variants of a nucleic acid sequence encoding an FGF-5 HLA-A2 or -A3 epitope are typically characterized by possession of at least 75%, at least 85%, at least 90%, at least 95%, at least 98%, or at least 99% sequence identity counted over full-length alignment with a nucleic acid sequence of encoding an FGF-5 HLA-A2 or -A3 epitope (such as a nucleic acid sequence encoding SEQ ID NO: 26-32, 34 or 39). However, nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that all encode substantially the same protein.

An alternative indication that two nucleic acid molecules are closely related is that the two molecules hybridize to each other under stringent conditions. Stringent conditions are sequence-dependent and are different under different environmental parameters. Conditions for nucleic acid hybridization and calculation of stringencies can be found in Sambrook et al. (In Molecular Cloning: A Laboratory Manual, CSHL, New York, 1988) and Tijssen (Laboratory Techniques in Biochemistry and Molecular Biology—Hybridization with Nucleic Acid Probes Part I, Chapter 2, Elsevier, New York, 1993). Non-limiting examples of hybridization conditions are provided below:

Very High Stringency (Detects Sequences that Share 90% Identity)

Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Detects Sequences that Share 80% Identity or Greater)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Detects Sequences that Share Greater than 50% Identity)
Hybridization: 6×SSC at RT to 55° C. for 16-20 hours
Wash at least twice: 2×-3×SSC at RT to 55° C. for 20-30 minutes each.

An alternative (and not necessarily cumulative) indication that two nucleic acid sequences are substantially identical is that the polypeptide which the first nucleic acid encodes is immunologically cross reactive with the polypeptide encoded by the second nucleic acid.

Subject: Living multicellular vertebrate organisms, a category which includes both human and veterinary subjects for example, mammals and birds.

T Cell: A white blood cell involved in the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one example, a CD8⁺ T cell is a cytotoxic T lymphocyte. In another example, a CD8⁺ cell is a suppressor T cell.

Therapeutically Effective Amount: An amount of a pharmaceutical preparation that alone, or together with a pharmaceutically acceptable carrier or one or more additional therapeutic agents, induces the desired response. A therapeutic agent, such as the disclosed pharmaceutical compositions that include an FGF-5 HLA-A2 or -A3 epitope (including protein or nucleic acid molecules, or CTLs specific to an FGF-5 epitope), is administered in therapeutically effective amounts.

Therapeutic agents (such as an FGF-5 epitope or CTLs specific to FGF-5) can be administered in a single dose, or in several doses, for example daily, weekly, monthly, or bimonthly during a course of treatment. However, the effective amount of can be dependent on the source applied (such as an FGF-5 epitope isolated from a cellular extract versus a chemically synthesized and purified epitope sequence, or a variant that does not retain full biological activity), the subject being treated, the severity and type of the condition being treated, and the manner of administration.

Effective amounts a therapeutic agent can be determined in many different ways, such as assaying for an increase in an immune response, for example by assaying for improvement of physiological condition of a subject having a disease (such as a tumor). Effective amounts also can be determined through various in vitro, in vivo or in situ assays. In one example, a therapeutically effective dose of FGF-5 epitope (such as a sequence that includes any of SEQ ID NOS: 26-32, 34, or 39) includes about 0.1 mg to about 10 mg of the peptide, such as about 1 mg of the peptide.

In one example, it is an amount sufficient to partially or completely alleviate symptoms of an FGF-5 expressing or overexpressing tumor. Treatment can involve only slowing the progression of the tumor temporarily, but can also include halting or reversing the progression of the tumor permanently, as well as preventing the tumor in the first place. For example, a pharmaceutical preparation can decrease one or more symptoms of the tumor (such as the size of the tumor or the number of tumors), for example decrease a symptom by at least 20%, at least 50%, at least 70%, at least 90%, at least 98%, or even at least 100%, as compared to an amount in the absence of the pharmaceutical preparation.

Transfected or Transformed: A transfected or transformed cell is a cell into which has been introduced a nucleic acid molecule by molecular biology techniques. As used herein, terms encompasses all techniques by which a nucleic acid molecule can be introduced into such a cell, such as viral vectors, plasmid vectors, and introduction of naked DNA by electroporation, lipofection, and particle gun acceleration.

Treating a disease: “Treatment” refers to a therapeutic intervention that ameliorates a sign or symptom of a disease or pathological condition, such a sign or symptom of an FGF-5 expressing or overexpressing tumor. Treatment can also induce remission or cure of a condition, such as an infectious disease or a tumor. In particular examples, treatment includes preventing a disease, for example by inhibiting the full development of a disease, such as preventing development or metastasis of an FGF-5 expressing or overexpressing tumor in a subject having an FGF-5 expressing tumor. Prevention of a disease does not require a total absence of infectious disease or a tumor. For example, a decrease of at least 50% can be sufficient.

Tumor: A neoplasm

Transgene: An exogenous nucleic acid sequence, for example supplied by a vector. In one example, a transgene encodes an FGF-5 HLA-A3 or HLA-A2 epitope.

Under conditions sufficient for: A phrase that is used to describe any environment that permits the desired activity.

In one example, includes administering an FGF-5 HLA-A3 or HLA-A2 epitope (either as a protein or a nucleic acid molecule) sufficient to allow the desired activity. In particular examples, the desired activity is the stimulation of an immune response. In other particular examples, the desired activity further includes treatment of an FGF-5 expressing or overexpressing tumor.

Variants or fragments or fusion proteins: Protein sequences which differ from a native or wild-type sequence, but substantially retain one or more biological functions of the protein. For example, the disclosed FGF-5 HLA-A3 and HLA-A2 epitopes include variants, fragments, and fusions thereof that retain the ability to stimulate an immune response.

In one example, an FGF-5 HLA-A3 and HLA-A2 epitope variant includes an FGF-5 HLA-A3 and HLA-A2 epitope protein sequence with one or more amino acid substitutions, such as one or more conservative substitutions, wherein the variant sequence retains the ability to stimulate an immune response.

A fusion antigen that includes an FGF-5 HLA-A3 or HLA-A2 epitope linked to other amino acids that do not significantly inhibit the ability to stimulate an immune response. In one example, the other amino acid sequences are at least 8, 9, 10, 12, 15, 20, 30, or 50 amino acid residues in length. In another example, the other amino acids are 1-50 amino acids, such as 1, 2, 3, 4, 5, 10, 20, 30, 40 or 50 amino acids.

A fragment of an FGF-5 HLA-A3 or HLA-A2 epitope includes an FGF-5 HLA-A3 and HLA-A2 epitope protein sequence with one or more amino acid deletions, for example from the N- or C-terminus, wherein the fragment substantially retains the ability to stimulate an immune response.

Vector: A nucleic acid molecule as introduced into a host cell, thereby producing a transformed host cell, thereby causing the cell to express the nucleic acid molecules and the proteins encoded thereby. A vector can include nucleic acid sequences that permit it to replicate in the host cell, such as an origin of replication. A vector can also include one or more therapeutic nucleic acid molecules (such as an FGF-5 HLA-A2 or -A3 epitope) or selectable marker genes and other genetic elements known in the art. A vector optionally includes materials to aid in achieving entry of the nucleic acid into the cell, such as a viral particle, liposome, protein coating or the like.

### FGF-5 HLA-A2 and -A3 Peptide and Nucleic Acid Molecules

The inventors have identified FGF-5 HLA-A2 and HLA-A3 immunogenic epitopes. Tumor infiltrating lymphocytes (TIL) were obtained from a metastatic pulmonary lesion from a renal cell carcinoma (RCC) patient who demonstrated a mixed spontaneous regression of lesions following nephrectomy. TIL were multiply restimulated with an autologous tumor cell line, expanded in IL-2, and cloned by limiting dilution. One CTL clone demonstrated HLA-A3 restricted recognition of autologous tumor, but did not recognize an autologous EBV-B cell line or an autologous fibroblast cell line. Clone 2 recognized a product of the fibroblast growth factor-5 (FGF-5) gene. An HLA-A2 epitipe was identified from HLA-A2 peripheral blood lymphocytes (PBL) stimulated with FGF-5 peptides from.

Therefore, the present application provides purified immunogenic FGF-5 HLA-A2 and HLA-A3 epitopes, as well a nucleic acid sequences that encode such epitopes.

Peptides

In a particular example, an FGF-5 HLA-A3 epitope is at least eight amino acids long, an in some instances is no more than 10 or 12 amino acids long, and includes the sequence Tyr-Ala-(A³)-(A⁴)-Arg-Phe wherein A³ is Ala or Ser and A⁴ is Ala or Pro (SEQ ID NO: 32). In one example, an immunogenic FGF-5 HLA-A2 or HLA-A3 epitope peptide includes, consists, or consists essentially of any amino acid sequence shown in any of SEQ ID NOS: 26-32, 34, and 39.

The disclosed FGF-5 HLA-A2 and HLA-A3 immunogenic peptides can be any length that permits the epitope to stimulate an immune response. In particular examples, an FGF-5 HLA-A2 or HLA-A3 epitope is at least 6 amino acids in length, such as at least 8, 9, 10, 11, 12, 15, 20 or even 30 amino acids in length, for example the epitope is 6-20 amino acids, 8-15 amino acids, 8-12 amino acids, 6-10 amino acids, or 9-10 amino acids in length. In another example, for example, the FGF-5 HLA-A2 and HLA-A3 epitopes disclosed herein is no more than 250 amino acids, such as no more than 100, 75, 50, 40, 30, 20 or even 15 amino acids. However, one skilled in the art will understand that fusion proteins including an FGF-5 HLA-A2 or HLA-A3 epitope can be even longer. For example, fusion proteins can include an FGF-5 HLA-A2 or HLA-A3 epitope with additional N- or C-terminal amino acids, such as at least 1, at least 5, at least 10 or at least 50 amino acids. In one example, a fusion protein that includes an FGF-5 HLA-A2 or HLA-A3 epitope is at least 80, at least 100, at least 120, at least 140, at least 160, at least 180, at least 200, at least 220 or at least 240 amino acids.

Particular examples of FGF-5 HLA-A2 and HLA-A3 epitopes are provided in SEQ ID NOS: 26-32, 34 and 39. However, the disclosure includes variants, fusions, and fragments of these sequences that substantially retain the ability to elicit or stimulate an immune response, for example in a subject having an FGF-5 expressing or over-expressing tumor. For example, SEQ ID NO: 34 is a functional fragment of SEQ ID NO: 26, and SEQ ID NO: 26 is a functional fragment of SEQ ID NO: 27.

Minor modifications of the amino acid sequence of the disclosed FGF-5 HLA-A2 and HLA-A3 epitopes can result in peptides which have substantially equivalent activity as compared to the unmodified counterpart peptide described herein. Such modifications can be deliberate, as by site-directed mutagenesis, or spontaneous. Peptides having such modifications are included herein as long as the biological activity of the variant peptide, such as the ability to induce an immune response or the ability to recognize the appropriate HLA-A2 or HLA-A3 expressing cell, still exists. In one example, a variant peptide has at least 78% sequence identity to any one of SEQ ID NOS: 26-32, 34 and 39, such as at least 80%, at least 89%, or at least 90% sequence identity. For example, functional variants of SEQ ID NO: 26 include the amino acid sequences shown in SEQ ID NOS: 28-31. In contrast, SEQ ID NO: 35 is not a functional variant of SEQ ID NO: 26 (and SEQ ID NO: 33 is not a functional variant of SEQ ID NO: 32).

Particular examples of variants include those having one or more conservative amino acid substitutions. In one example, a variant includes a single amino acid substitution, such as a single conservative amino acid substitution in any of SEQ ID NOS: 26-32, 34 and 39. In a particular example, a variant includes two conservative amino acid substitutions, or three conservative amino acid substitutions, for example in any of SEQ ID NOS: 26-32, 34 and 39.

Determining whether a variant, fragment, or fusion of a disclosed FGF-5 HLA-A2 or HLA-A3 epitope can be determined by particular methods disclosed herein. However, the disclosure is not limited to the particularly described examples. In one example, a T-cell that recognizes an FGF-5 HLA-A2 or HLA-A3 epitope can also recognize a variant of the FGF-5 HLA-A2 or HLA-A3 epitope of interest. In another example, a variant FGF-5 HLA-A2 or HLA-A3 immunogenic peptide can stimulate propagation of an immune cell (such as a T-cell). In another example, a variant FGF-5 HLA-A2 or HLA-A3 is immunogenic and can elicit an immune response against an FGF-5 expressing tumor in a subject, such as those subjects having an HLA-A2 allele, an HLA-A3 allele, or both.

The disclosed peptides can be purified using standard techniques. In one example, substantially pure peptides yield a single major band on a non-reducing polyacrylamide gel. The peptide can be purified from the gel. In another example, a recombinantly expressed peptide is performed using preparative chromatography and immunological separations using antibodies (for example an FGF-5 antibody that recognizes the epitope or an antibody that recognizes a tag on the epitope, such as a His tag). The purity of a peptide can be determined by N-terminal sequence analysis.

Nucleic Acid Molecules

Also provided by the present disclosure are FGF-5 HLA-A2 and HLA-A3 epitope nucleic acid sequences, such as those that encode the disclosed peptides. An example of a nucleic acid sequence that encodes an FGF-5 HLA-A2 epitope includes nucleotides 488-517 of SEQ ID NO: 3. An example of a nucleic acid sequence that encodes an FGF-5 HLA-A3 epitope includes nucleotides 663-667 and 788-799 of SEQ ID NO: 3 operably linked to each other. Variants of such sequences, such as those sharing at least 70%, at least 80%, at least 90%, or at least 95% sequence identity to 488-517 of SEQ ID NO: 3 or to nucleotides 663-667 and 788-799 of SEQ ID NO: 3 operably linked to each other, are encompassed by this disclosure.

Examples of such nucleic acid molecules include DNA, cDNA and RNA sequences. The nucleotides can be ribonucleotides, deoxyribonucleotides, or modified forms of either nucleotide. It is understood that all nucleic acid sequences that encode an FGF-5 HLA-A2 or -A3 epitope are included herein, as long as they encode a peptide with the desired biological activity, such as the ability to induce an immune response or the ability to recognize the appropriate HLA-A2 or HLA-A3 expressing cell. In particular examples, an FGF-5 HLA-A2 or HLA-A3 epitope nucleic acid sequence is at least 15, 24, 27, 30, 50, 100, 200, 500, 1000, or 5000 nucleotides, such as 20-50 nucleotides, 18-36 nucleotides, or 27-30 nucleotides, and also includes full length cDNAs. However, nucleic acid sequences that include an FGF-5 HLA-A2 or HLA-A3 epitope nucleic acid sequences, such as a vector, can be longer.

One of ordinary skill in the art will appreciate that an FGF-5 HLA-A2 or -A3 nucleic acid sequence can be altered in numerous ways without affecting the biological activity of the encoded protein. For example, variants can be variants optimized for codon preference in a host cell used to express the protein, or other sequence changes that facilitate expression. In addition, nucleic acid sequences include sequences that are degenerate as a result of the genetic code encode FGF-5 HLA-A2 or HLA-A3 epitope sequences that are unchanged. Therefore, FGF-5 HLA-A2 and -A3 nucleic acid sequences encoding the disclosed peptides include the disclosed sequences, degenerate sequences, and sequences that encode FGF-5 HLA-A2 and -A3 variants, fragments, and fusions thereof.

The nucleic acid sequences encoding an FGF-5 HLA-A2 or HLA-A3 epitope (or variant, fragment or fusion thereof) can be inserted into an expression vector, such as a vector that contains a promoter sequence to facilitate transcription of the inserted epitope sequence. Expression vectors typically contain an origin of replication, a promoter, as well as specific nucleic acid sequences that allow phenotypic selection of the transformed cells. Examples of vectors include, but not are not limited to, a plasmid, virus or other vehicle that has been manipulated by insertion or incorporation of an FGF-5 HLA-A2 or HLA-A3 nucleic acid sequence. FGF-5 HLA-A2 or HLA-A3 epitope nucleic acid sequences can also be inserted into the genomic DNA of a prokaryote or eukaryote, or can exist as a separate molecule (such as a cDNA) independent of other sequences in a cell. Therefore, the disclosure provides FGF-5 HLA-A2 and HLA-A3 epitope nucleic acid sequences that are part of a vector, as well as cells that include such vectors.

Also encompassed by the disclosure are fragments of the above-described nucleic acid sequences that are at least 15 bases in length, such as at least 27 bases, such as at least 30 bases, which are sufficient to permit the fragment to selectively hybridize to DNA that encodes the disclosed FGF-5 HLA-A2 or HLA-A3 epitope (such as a nucleic acid molecule that encodes any one of SEQ ID NOS: 26-32, 34 and 39, or nucleotide sequences that include such sequences) under physiological conditions. The term “selectively hybridize” refers to hybridization under moderately or highly stringent conditions which excludes non-related nucleotide sequences.

DNA sequences encoding a disclosed FGF-5 HLA-A2 or HLA-A3 epitope can be expressed by DNA transfer into a suitable host cell. The cell can be prokaryotic or eukaryotic (such as microbial, yeast, insect and mammalian cells). The term also includes any progeny of the subject host cell. It is understood that all progeny may not be identical to the parental cell since there may be mutations that occur during replication. Methods of transferring and expressing exogenous nucleic acid sequences are known. To obtain expression, the DNA sequence can be altered and operably linked to other regulatory sequences. The final product, which contains the regulatory sequences and the therapeutic protein, is referred to as a vector. This vector can be introduced into eukaryotic or prokaryotic cells. Once inside the cell the vector allows the protein to be produced.

FGF-5 HLA-A2 or HLA-A3 epitope nucleic acid sequences can be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (such as ATG) in front of a protein-encoding gene, splicing signal for introns, maintenance of the correct reading frame of that gene to permit proper translation of mRNA, and stop codons. The term “control sequences” is intended to included, at a minimum, components whose presence can influence expression, and can also include additional components whose presence is advantageous, for example, leader sequences and fusion partner sequences. Expression control sequences can include a promoter.

Promoters include a minimal sequence sufficient to direct transcription. Promoter-dependent gene expression can be used to control cell-type specific expression, tissue-specific expression, or inducible by external signals or agents expression. Promoters can be located in the 5′ or 3′ regions of the gene. Both constitutive and inducible promoters, can be used (Bitter et al., Meth. Enzymol. 153:516-44, 1987). For example, when cloning in bacterial systems, inducible promoters such as pL of bacteriophage γ, plac, ptrp, ptac (ptrp-lac hybrid promoter) and the like can be used. When cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences of the disclosure.

### FGF-5 HLA-A2 and HLA-A3 Epitopes as Immunogenic Compositions

The present disclosure also provides immunogenic compositions that include FGF-5 HLA-A2 or -A3 nucleic acid molecules or proteins (as well as variants, fragments, and fusions thereof, such as those described above), immunoreactive sensitized T cells sensitized with an FGF-5 epitope, or combinations thereof. Such compositions can be administered to a subject to elicit or stimulate an immune response in a subject or for treating an FGF-5 expressing or over-expressing tumor in a subject. In a particular example, a pharmaceutical composition includes a therapeutically effective amount of both an FGF-5 HLA-A2 epitope and an FGF-5 HLA-A3 epitope.

The pharmaceutical compositions disclosed herein can be administered prophylactically, prior to the development of an FGF-5 expressing or over-expressing tumor, for example to persons at elevated risk of developing the tumor (such as persons with a family history of the tumor, and persons with Hippel-Lindau disease, horseshoe kidneys, adult polycystic kidney disease, and acquired renal cystic disease). The pharmaceutical compositions can also be administered therapeutically to a subject who already has an FGF-5 expressing or over-expressing tumor, for example a subject who has undergone surgical resection of a RCC primary or metastatic lesion, or a person who is undergoing chemotherapy for treatment of the tumor. Alternatively, the disclosed compositions can be administered as the sole therapy for the tumor.

The disclosed pharmaceutical compositions for eliciting an immune response in a subject can include a therapeutically effective amount of one or more FGF-5 HLA-A2 or HLA-A3 epitopes, immunoreactive sensitized T cells sensitized with one or more FGF-5 epitopes, or combinations thereof, alone or in combination with other agents. For example, the pharmaceutical compositions can include one or more pharmaceutically acceptable carriers, such as an adjuvant (for example Montanide ISA-51). Adjuvants are nonspecific immune stimulators that when used in combination with an immunogenic agent augments or otherwise alters a resultant immune response. In another example, the pharmaceutical compositions include one or more additional therapeutic agents, such as a therapeutically effective amount of one or more anti-neoplasic agents (for example IL-2). The disclosed pharmaceutical compositions can be administered concurrently or sequentially with the other therapeutic agents.

In one example, the FGF-5 HLA-A2 and -A3 epitopes, such as SEQ ID NOS: 26-32, 34, and 39, present in the immunogenic composition are obtained from natural sources. In this example, an FGF-5 protein is subjected to selective proteolysis, for example by splitting the protein with chemical reagents or enzymes, to obtain the desired epitope fragment. In another example, because the disclosed FGF-5 HLA-A2 and -A3 epitopes, such as SEQ ID NOS: 26-32, 34, and 39, are relatively short, the peptides can be synthesized in solution or on a solid support in accordance with conventional techniques. Various automatic peptide synthesizers are commercially available and can be used in accordance with known protocols. Chemical synthesis of peptides is described in: S. B. H. Kent, Biomedical Polymers, eds. Goldberg and Nakajima, Academic Press, New York, pp. 213-242, 1980; Mitchell et al., J. Org. Chem., 43:2845-52, 1978; Tam et al., Tet. Letters, 4033-6, 1979; Mojsov et al., J. Org. Chem., 45:555-60, 1980; Tam et al., Tet. Letters, 2851-4, 1981; and Kent et al., Proceedings of the IV International Symposium on Methods of Protein Sequence Analysis, (Brookhaven Press, Brookhaven, N.Y., 1981. In addition, recombinant DNA technology can be employed wherein a nucleotide sequence which encodes one or more epitopic peptides is inserted into an expression vector, transformed or transfected into an appropriate host cell and cultivated under conditions suitable for expression, as disclosed herein.

The length of the amino acid sequence produced can depend on the method of producing the sequence. If the sequence is made by assembling amino acids by chemical means, the sequence in particular examples does not exceed, for example, 50, 40, or 30 amino acids. In one example, an FGF-5 HLA-A2 or -A3 epitope peptide sequence generated using chemical synthesis is 6-50 amino acids in length, such as 6-20, 6-12, 6-10, 8-10, or 9-10 amino acids. If the synthetic peptide is made by translating a nucleic acid molecule, the peptide can be any length, for example, 100 amino acids or more. However, the peptide can also be shorter, for example, no more than 50, no more than 40, no more than 20, no more than 10, no more than 9, or no more than 8 amino acids, such as 6-50 amino acids, 6-20, 6-12, 6-10, 8-10, or 9-10 amino acids.

FGF-5 HLA-A2 and HLA-A3 epitope variants, fragments, and fusions can be employed in the pharmaceutical compositions, and can include one or more amino acid additions, one or more amino acid deletions, one or more amino acid replacements, one or more isostereomer (a modified amino acid that bears close structural and spatial similarity to the original amino acid) substitutions, one or more isostereomer additions, or combinations thereof, wherein the altered sequence is recognized by, or can generate, an immune cell. For example, a variant of SEQ ID NO: 26-32, 34, or 39, will be recognized by an immune cell that recognizes SEQ ID NO: 26-32, 34, or 39, respectively. In a particular example, such variants, fragments, and fusions, provide an advantage, such as increasing the solubility or immunogenicity of the epitope, or easing linking or coupling of the epitope. In one example, the peptides included in the pharmaceutical composition can form neutralizing antibodies to an FGF-5 HLA-A2 or HLA-A3 epitope.

The disclosed FGF-5 HLA-A2 and HLA-A3 epitopes can also be engineered to include other amino acids (to generate a fusion protein), such as residues of various moieties, such as additional amino acid segments or polysaccharides. Examples include, but are not limited to, moieties which augment or induce antigen processing, epitope stability or manufacture, or delivery within the body to sites appropriate for immunization or recognition by immune cells. In addition, an amino acid chain corresponding to an additional antigen or immunogen can be included. Thus, an immune response to more than one antigen can be induced by immunization. Specific non-limiting examples of antigens or immunogens include, but are not limited to, tumor antigens of other tumor antigens from FGF-5-expressing cancers which may increase or provoke CD4⁺ T-cell (helper T-cell) responses supportive of an FGF-5 immune response. These additional amino acid sequences can be of varying length, such as at least 5 amino acids, at least 10 amino acids, at least 25 amino acids, at least 50 amino acids, at least 100 amino acids, or no more than 500 amino acids, such as no more than 250 amino acids, no more than 100 amino acids, no more than 75 amino acids, no more than 50 amino acids, no more than 25 amino acids, no more than 15 amino acids, or no more than 10 amino acids, such as 5-50 amino acids.

In some examples, it is desirable to combine two or more FGF-5 HLA epitopes that contribute to stimulating specific immune responses in one or more subjects or histocompatibility types. The epitopes in the composition can be identical or different, and together they can provide equivalent or greater biological activity than the parent epitopes(s). For example, multiple epitopic peptides can be combined in a “cocktail” to provide enhanced immunogenicity, and peptides can be combined with peptides having different MHC specificities. Such compositions can be used to effectively broaden the immunological coverage provided by the disclosed immunogenic compositions.

In some examples, epitopic peptides are linked with or without a spacer molecule to form polymers (multimers), or can be formulated in a composition without linkage, as an admixture. Where the same peptide is linked to itself, thereby forming a homopolymer, a plurality of repeating epitopic units are presented. When the peptides differ, heteropolymers with repeating units are provided.

Linkages for homo- or hetero-polymers or for coupling to carriers and adjuvants can be provided in a variety of ways, such as through covalent linkages between epitopic peptides or noncovalent linkages capable of forming intermolecular and intrastructural bonds. When present, the spacer can include relatively small, neutral molecules, such as amino acids or amino acid mimetics, which are substantially uncharged under physiological conditions and may have linear or branched side chains. Particular examples of spacers include on or more alanine or glycine residues, or other nonpolar amino acids or neutral polar amino acids. Spacers can be either homo- or hetero-oligomers and can include one or two residues, more typically three to six residues. Spacers can be attached to epitopic peptides at the C-terminus, N-terminus or a side chain of one or more of the amino acids. Examples of crosslinking agents that can be used to interconnect a plurality of epitopes include crosslinking agents having an aldehyde (such as glutaraldehyde), carboxyl, amine, amido, imido or azidophenyl functional group. In a particular example, butyraldehyde is used as a crosslinking agent, a divalent imido ester or a carbodiimide.

In another example, cysteine residues can be added at the amino- and carboxy-termini to permit formation of bonds between peptides via controlled oxidation of the cysteine residues. Heterobifunctional agents, which generate a disulfide link at one functional group end and a peptide link at the other, including N-succidimidyl-3-(2-pyridyldithio) proprionate (SPDP) can also be employed. A variety of such disulfide/amide forming agents are known (For example, Immun. Rev. 62:185, 1982). Other bifunctional coupling agents form a thioether rather than a disulfide linkage. Many of these thioether forming agents are commercially available and include reactive esters of 6-maleimidocaproic acid, 2 bromoacetic acid, 2-iodoacetic acid, and 4-(N-maleimido-methyl) cyclohexane-1-carboxylic acid. In these reagents, the carboxyl groups can be activated by combining them with succinimide or 1-hydroxy-2-nitro-4-sulfonic acid, sodium salt. One coupling agent is succinimidyl 4-(N-maleimidomethyl) cyclohexane-1-carboxylate (SMCC). Ideally, the linkage does not substantially interfere with the immunogenicity of the linked epitopic peptides.

A particular example of a fusion protein, which includes one or more FGF-5 HLA-A2 or -A3 epitopic peptide sequences, can be used to present the epitopic peptides to a subject. For example, a recombinant HBV surface antigen protein is prepared in which the HBenv amino acid sequence is altered so as to more effectively present epitopes of peptide regions described herein to stimulate an immune response. By this means a polypeptide may incorporate several epitopes. Coding sequences for peptides of the length contemplated herein can be synthesized by chemical techniques, for example, the phosphotriester method of Matteucci et al. (J. Am. Chem. Soc. 103:3185, 1981). The coding sequence can then be provided with appropriate linkers and ligated into expression vectors commonly available in the art, and the vectors used to transform suitable hosts to produce the desired fusion protein as disclosed herein and known in the art.

### Methods of Stimulating an Immune Response

Methods are disclosed for eliciting or stimulating an immune response in a subject. In particular examples, such methods can be used to treat a subject having an FGF-5 expressing or overexpressing tumor. FGF-5 is expressed in several tumor cells, such as RCC (Yoshimura et al., Cancer Lett. 103:91-7, 1996), and cancers of the bladder, liver and endometrium (Zhan et al., Mol Cell Biol. 8:3487-95, 1988; the '916 and '217 patents; Yoshimura et al., Cancer Lett. 103:91-7, 1996), pancreas (Kommann et al., Oncogene. 15:1417-24, 1997), gastric and esophageal adenocarcinomas (Altorki et al., Cancer, 72:649-57, 1993), breast (Cullen et al., Cancer Res. 51:4978-85, 1991) and malignant melanomas (Albino et al., Cancer Res. 51:4815-20, 1991). However, whether FGF-5 expression in these tissues correlated with the immunogenicity of FGF-5 was not previously known, and therefore it was not known whether FGF-5 could be used as an immune target for cancer immunotherapies.

In particular examples, the method includes administering to a subject a therapeutically effective amount of one or more FGF-5 HLA-A2 or HLA-A3 epitopes (or a nucleic acid molecule encoding such an epitope), immunoreactive sensitized T cells sensitized with an FGF-5 epitope, or combinations thereof, thereby eliciting an immune response in a subject. Examples of FGF-5 HLA-A2 or HLA-A3 peptides (for example SEQ ID NOS: 26-32, 34, and 39), and the corresponding nucleic acid sequences, as well as variants, fragments, and fusions thereof, are disclosed throughout the application. For example, an FGF-5 HLA-A2 or HLA-A3 epitope having no more than 15 or 20 amino acids, such as 6-12 amino acids, can be administered to the subject directly, or with a pharmaceutically acceptable carrier. In another example, a nucleic acid molecule that includes a sequence that encodes an FGF-5 HLA-A2 or HLA-A3 epitope that can stimulate a cytotoxic T cell response (for example against a cell of the FGF-5 expressing or overexpressing tumor) is administered to the subject. Such a nucleic acid sequence can be part of a vector, for example a viral vector, such as a retroviral vector sufficient to stimulate a cytotoxic T cell response.

Therefore, a therapeutically effective amount of FGF-5 HLA-A2 or HLA-A3 epitope peptide (including variants, fusions, and fragments thereof) that elicits or stimulates an immune response can be administered directly to the subject, or can be delivered to the subject by administering a therapeutically effective amount of a recombinant nucleic acid encoding an FGF-5 HLA-A2 or HLA-A3 epitope. In another example, the subject administered a therapeutically effective amount of immunoreactive sensitized T cells, wherein the sensitized T cells are sensitized with an FGF-5 HLA-A2 or HLA-A3 epitope. The immunoreactive sensitized T cells can be autologous or heterologous. For example, T cells can be obtained from a subject (for example from a blood sample), incubated with an FGF-5 HLA-A2 or HLA-A3 epitope under conditions sufficient for sensitizing the T cells (for example sufficient to stimulate the T cell to react with a cell of an FGF-5 expressing or overexpressing tumor), and then introducing the sensitized T cells into the same or another subject.

Specific, non-limiting examples of an immune response are a B cell or a T cell response (or both). For example, the immune response can be the generation of antibodies specific for an FGF-5 HLA-A2 or HLA-A3 antigen. In another example, the immune response is a T cell response, for example an increase in the number of CD4 or CD8 cells or the ability of a T cell to react with a cell of an FGF-5 expressing neoplasm or tumor. For example, the immune response stimulates a cytotoxic T cell response to a cell of an FGF-5 expressing or overexpressing neoplasm, and thereby decrease or even inhibit neoplastic proliferation. In some examples, decreasing neoplastic proliferation induces a regression of the tumor, for example as determined by radiographic or other laboratory evidence of disease (such as serologic tumor markers).

In particular examples, the disclosed methods are used to treat an FGF-5 expressing or over-expressing tumor, for example by halting progression of the tumor, by causing regression of the tumor, retarding growth of the tumor, decreasing or preventing metastasis of the tumor, or combinations thereof. Examples of FGF-5 expressing or over-expressing tumors include, but are not limited to, adenocarcinomas, such as RCC or adenocarcinomas of the breast, prostate, pancreas and bladder. In specific examples, the neoplasm is a RCC. Therefore, in some examples, the method can also include determining whether a subject's tumor expresses or overexpresses FGF-5. Methods for making such a determination are known in the art.

In particular examples, the method of increasing an immune response is used to lyse a cell of an FGF-5 expressing or overexpressing neoplasm by FGF-5, for example to induce regression of the neoplasm.

Haplotyping the Subject

In one example, the HLA haplotype of the subject, such as a human subject, is determined prior to administering a therapeutically effective amount of FGF-5 HLA-A2 or HLA-A3 epitope. This allows one, such as a physician, to determine the appropriate FGF-5 epitope to administer to the subject, wherein the epitope administered matches the hapotype of the individual to whom it is given. For example, if the subject is determined to have an HLA-A2 haplotype (that is they have at least one HLA-A2 allele), the subject can be administered an FGF-5 HLA-A2 epitope (such as a sequence that includes SEQ ID NO: 32 or a variant, fragment or fusion thereof). Similarly, if the subject is determined to have an HLA-A3 haplotype (that is they have at least one HLA-A3 allele), the subject can be administered an FGF-5 HLA-A3 epitope (such as one or more of SEQ ID NOS: 26-31, 34, and 39).

Methods for haplotyping a subject are known. Although particular examples are provided herein, the disclosure is not limited to these methods. In one example, the method for haplotyping uses the polymerase chain reaction (PCR) to compare the DNA of the person, with known segments of the genes encoding MHC antigens. The variability of these regions of the genes determines the tissue type or haplotype of the subject.

Serologic methods can be used to detect serologically defined antigens on the surfaces of cells. HLA-A, -B, and -C determinants can be measured by known serologic techniques. Briefly, lymphocytes from the subject (isolated from fresh peripheral blood) are incubated with antisera that recognize all known HLA antigens. The cells are spread in a tray with microscopic wells containing various kinds of antisera. The cells are incubated for 30 minutes, followed by an additional 60-minute complement incubation. If the lymphocytes have on their surfaces antigens recognized by the antibodies in the antiserum, the lymphocytes are lysed. A dye can be added to show changes in the permeability of the cell membrane and cell death. The proportion of cells destroyed by lysis indicates the degree of histologic incompatibility. If, for example, the lymphocytes from a person being tested for HLA-A3 are destroyed in a well containing antisera for HLA-A3, the test is positive for this antigen group.

### EXAMPLE 1

### Isolation of Tumor Infiltrating Lymphocytes (TILS)

This example describes the isolation of TILs from a metastatic RCC lung lesion. Similar methods can be used to isolate TILs from other neoplasms.

The surgically resected remnant of a spontaneously regressing metastatic RCC lung lesion was enzymatically digested (0.1% Collagenase type IV, 30 u/ml deoxyribonuclease type IV, and 0.01% hyaluronidase type V [Sigma, St. Louis, Mo.]) at room temperature (RT) for three hours. After digestion, the cell suspension was filtered through 100 μm nylon mesh and separated by density gradient using Lymphocyte Separation Medium (Organon Teknica, Durham, N.C.). The lymphocyte-containing interface was recovered, washed with Hanks' Balanced Salt Solution, and used for TIL culture in RPMI (Biofluids, Rockville, Md.) 10% human AB serum (Biochemed Pharmacologicals, Winchester, Va.) with 6000 IU/ml of recombinant human IL-2 (Chiron Corp., Emoryville, Calif.) in 24 well plates at a cell density of 1×10⁶ cells/well. The culture was stimulated every two weeks with an irradiated autologous RCC cell line established from a primary left nephrectomy sample (EXAMPLE 2).

An autologous EBV-B cell line was established by EBV infection using a culture supernatant of cell line B95-9 (American Type Culture Collection, ATCC, Manassas, Va.). An autologous fibroblast line was established by infecting a one-week old cultured tumor sample with a retrovirus that encoded the papilloma virus E6/E7 proteins and performing limiting dilution. RCC cell lines UOK125, UOK127, UOK130, UOK131, UOK150, and UOK 171 were obtained from Dr. W. Marston Linehan (NCI, Bethesda, Md.). Lung cancer cell lines SKGT2, SKGT4, SKGT5 and esophageal cancer cell line HCE-4 were obtained from Dr. David Schrump (NCI, Bethesda, Md.). 1570 RCC, 1581 RCC, 1645 RCC, 1764 RCC, CY13, 501 mel, 526 mel, 586 mel, 624.38 mel, 888 mel, 1088 mel, 1199 mel, and 1479 mel were established from surgical samples. SW480, PC-3, and DU 145 were obtained from ATCC (Manassas, Va.). TSU-PR1 was obtained from Dr. Suzanne Topalian (NCI, Bethesda, Md.).

### EXAMPLE 2

### Identification of a CTL Clone with Specific Recognition of Autologous RCC

One of the regressing remnants of a metastatic lung lesion of a renal cell carcinoma produced a TIL line when cultured in media with IL-2. This bulk TIL line was stimulated periodically with an irradiated autologous tumor cell line established from a left nephrectomy operation sample. After three months of culture, the autologous tumor-specific T cell clone (Clone 2 CTL) was established by limiting dilution of the bulk TIL line. Clone 2 CTL was CD3⁺, CD8⁺ and was found to utilize Vp 12 by PCR-based TCR (T-cell receptor) analysis.

The reactivity of Clone 2 CTL was assessed using an autologous RCC cell line (1764 CTL), an autologous EBV-B cell line, an autologous fibroblast cell line, and other allogeneic RCC cell lines described in EXAMPLE 1. RCC cells (5×10⁴ cells/well), fibroblasts (5×10⁴ cells), or EBV-B cells (1×10⁶ cells/well) were plated into 96 well flat bottom plates and 2×10⁴ cells/well of Clone 2 CTL were added. After incubating for 20 hours, supernatants were harvested and IFN-γ concentration was analyzed by ELISA (Endogen, Woburn, Mass.), where IFN-γ concentration is considered proportional to CTL activation.

ELISA plates (96 well flat bottom, Costar, NY) were coated with anti-human IFN-γ monoclonal antibody (2G1, ENDOGEN, MA) at 1 μg/ml, 100 μl/well overnight. After washing, plates were blocked with PBS-5% FBS (fetal bovin serum) for one hour (200 μl/well), the samples added (100 μl/well) and incubated for 90 minutes. Subsequently, the plates were washed and biotin-labeled anti-human IFN-γ monoclonal antibody (B133.5, ENDOGEN, MA) was added at 0.5 μg/ml, 100 μl/well, and incubated for one hour. The plates were subsequently washed and 2000-times diluted HRP-streptavidin conjugate (Zymed Laboratories, CA) was added and incubated for 30 minutes. Plates were washed and DAKO TMB One-Step Substrate System (DAKO Corporation, CA) was added (100 μl/well). The coloration reaction was stopped by adding 0.18 M sulfuric acid (100 μl/well) and the optical density at 450 nm wave-length was measured. Recombinant human IFN-γ (ENDOGEN, MA) was used as a standard.

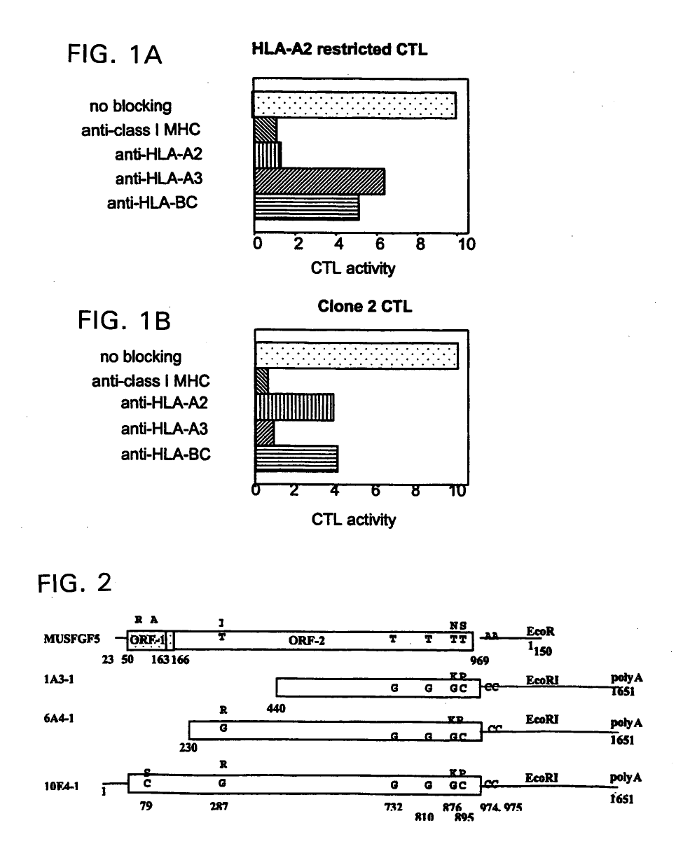

The reactivity of Clone 2 CTL with a panel of HLA-typed tumors indicated that Clone 2 CTL recognized an antigen shared among renal cell carcinomas (RCC), and this recognition appeared to be restricted by HLA-A3. To confirm the restriction by HLA-A3, a blocking study using HLA-specific monoclonal antibodies (mAbs) was performed. W6/32, MA2.1, and GAPA3 hybridomas were obtained from ATCC and the antibodies purified by Lofstrand Labs (Gaithersburg, Md.). B1.23.1 was obtained from NCI, Bethesda, Md. Irradiated tumor cells (5×10⁴ cells in 100 μl) were incubated with mAbs (20 μg/ml) for 30 minutes at RT, and 2×10⁴ CTL were added. Following 20 hours of culture at 37° C., the supernatant was assayed for IFN-γ concentration by ELISA as described above. As a control (FIG. 1A), a CTL and autologous tumor target whose interaction is known to be restricted by HLA-A2 was used. The effect of a blocking mAb on the recognition of autologous tumor by Clone 2 CTL is shown in FIG. 1B (anti-class I MHC mAb=W6/32, anti-HLA-A2 mAb=MA2.1, anti-HLA-A3 mAb=GAPA3, anti-HLA-BC mAb=B 1.23.1).

As shown in FIG. 1B, tumor recognition by Clone 2 CTL was maximally reduced by a pan-anti-class I MHC mAb (W6/32) and an anti-HLA-A3 mAb (GAPA3). At least 75% inhibition was observed, when compared to the CTL activity of the HLA-A2 restricted CTL. However, blocking by an anti-HLA-A2 mAb (MA2.1) and an anti-HLA-B/C mAb (B1.23.1) was similar to the effect observed with the anti HLA-B/C antibody on an HLA-A2-restricted CTL. Therefore, Clone 2 CTL reactivity is restricted by HLA-A3.

### EXAMPLE 3

### Cloning the Antigen Recognized by Clone 2 CTL

To identify the gene coding for the antigen recognized by Clone 2 CTL, expression cloning was performed utilizing a plasmid-based cDNA library. Poly (A)⁺ RNA was prepared from the autologous RCC cell line using a mRNA isolation system (Fast Track kit 2.0; Invitrogen, Carlsbad, Calif.). cDNA was prepared with the Superscript plasmid system (Life Technologies, Rockville, Md.) and ligated into the eukaryotic plasmid expression vector pME18S (Dr. Atsushi Miyajima, University of Tokyo). After electroporation and titration, a cDNA library was prepared in pools by inoculating approximately 100 bacterial clones/well in 1 ml LB/well and purifying plasmid using the Qiaprep 96 turbo system (Qiagen, Valencia, Calif.).

To serve as the antigen-presenting target cell line, COS-7 cells were retrovirally transduced with the HLA-A3 gene derived from the autologous tumor cell line. As a control target, the HLA-A0201 gene was retrovirally transduced into COS-7 cells (hereafter referred to as COS-A3 or COS-A2, respectively). To introduce HLA-A3 into non-HLA-A3-expressing cells, the HLA-A3 gene was cloned from autologous 1764 RCC by RT-PCR (forward primer 5′-TTGGGGAGGGAGCACAGGTCAGCGTGGGAAG-3′, SEQ ID NO: 24; reverse primer 5′-GGACTCAGAATCTCCCCAGACGCCGAG-3′, SEQ ID NO: 25), sequenced, and subcloned into the retroviral vector pRx-IRES-Bsr (Wakimoto et al., Jpn. J. Cancer Res. 88:296-305, 1997). Vesicular stomatitis virus G protein-pseudotyped retrovirus was prepared by transiently transfecting the 293 GP cell line using standard methods (see Wang et al., Cancer Res. 58:3519-25, 1998, incorporated by reference). Forty-eight hours after transfection, culture supernatant was harvested, filtered, and used for infection with 8 μg/ml of polybrene. Infection efficiencies ranged from 70%-100% and the HLA-A3 positive population was further selected by 5 μg/ml of blasticidin S (Calbiochem, San Diego, Calif.).

Three independent clones: IA3 (SEQ ID NOS: 7 and 8), 6A4 (SEQ ID NOS: 11 and 12), and 10E4 (SEQ ID NOS: 13-18) selected after subcloning reactive cDNA pools were transfected (100 ng of plasmid) into 5×10⁴ COS-A2 and COS-A3 using 1 μl of lipofectAMINE (Life Technologies, Rockville, Md.) in 96-well plates according to the manufacturer's instructions. The next day, Clone 2 CTL (2×10⁴ cells/well) was added and after 20 hours incubation, supernatants were assayed for IFN-γ secretion by Clone 2 CTL as described in the above EXAMPLES. An FGF-5 gene from an independent source (FGF-5 MG, from Dr. Mitchell Goldfarb, Mount Sinai School of Medicine) was analyzed the same way.

The measured IFN-γ concentration was greater than 2500 pg/ml for the COS-A3 cells, but less than 1 pg/ml for the COS-A2 cells. The clones that conferred recognition of COS-A3 by the CTL were identified and sequenced on an automated sequencer (ABI Prizm 310; Perkin-Elmer Corp., Foster City, Calif.).

All three clones encoded all or part of an FGF-5 sequence. As shown in FIG. 2, the sequence of the clones is similar, but not identical to, the human FGF-5 sequence from GenBank Accession No M37825 (MUSFGF5A; SEQ ID NOS 1-4). There were eight nucleotide mismatches at positions 79 (A→C), 287 (T→G), 732 (T→G), 810 (T→G), 876 (T→G), 895 (T→C), 974 (A→C) and 975 (ARC), four of which resulted in amino acid changes. The smallest cDNA clone (FIG. 2) recognized by Clone 2 CTL (1A3-1) (SEQ ID NOS: 7 and 8) had six nucleotide mismatches and two of these changed the amino acid sequence. The longest clone (10E4-1) contained the full-length FGF-5 cDNA sequence (SEQ ID NOS: 15 and 17).

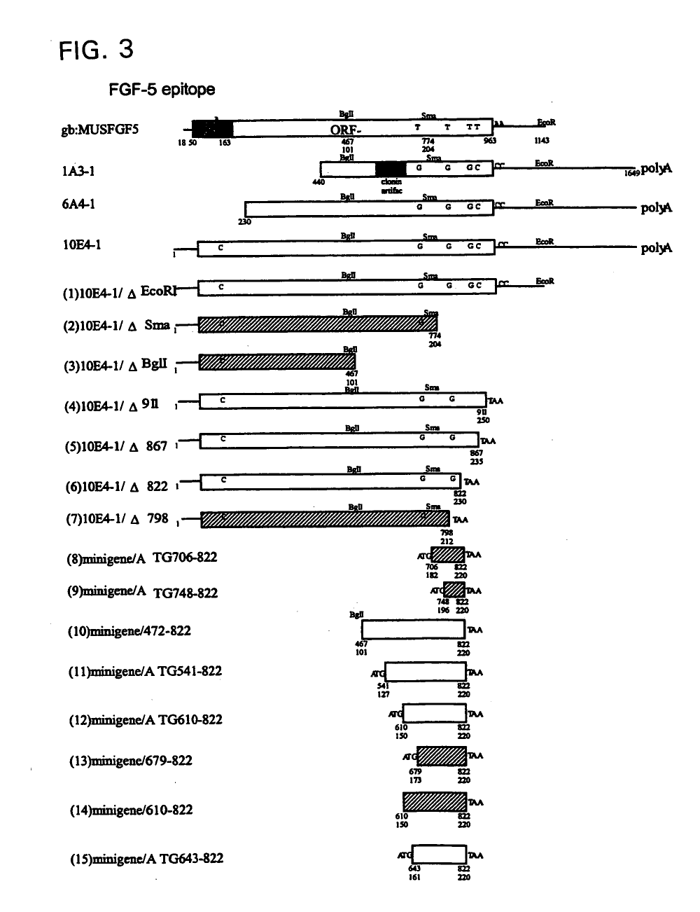

The genomic sequence for FGF-5 obtained from autologous EBV-B cells was identical to the autologous tumor-derived sequence for FGF-5. In addition, FGF-5 cDNA from an independent source (FGF-5 MG) was also recognized by Clone 2 CTL when transfected into COS-A3 (FIG. 3A). The DNA sequence of the FGF-5 MG plasmid was identical to clone 1A3-1 (SEQ ID NO: 7). Therefore, the previously published FGF-5 sequence available on GenBank Accession No. M37825 contains at least eight nucleotide and four amino acid differences.

These data demonstrate that Clone 2 CTL recognizes a non-mutated epitope within the 268 amino acid full-length FGF-5. Fragments of FGF-5 were also shown to activate CTL clonal expansion. Fragments of FGF-5 tested are shown in FIG. 3. Fragments which activated CTL clonal expansion are shown in FIG. 3 as open bars, while fragments which did not activate CTL clonal expansion are shown in FIG. 3 as filled bars. Fragment 610-822 did not activate an immune response, while a fragment as short as 60 amino acid residues, 643-822, (SEQ ID NO: 19) are immunogenic in HLA-A3 individuals. The region of amino acids 643-679 may be modified in cells. The disclosure therefore provides a number of species of immunogenic peptides, and permits one to easily screen for other immunogenic peptides with the assay of this example.

### EXAMPLE 4

### Analysis of FGF-5 Expression in Normal and Tumor Cells

To analyze FGF-5 expression, real-time-PCR (RT-PCR) analysis and INF-γ release was measured in normal cells, tumor cells naturally expressing HLA-A3, and in non-HLA-A3 expressing tumors in which HLA-A3 was introduced by retroviral transduction (see EXAMPLE 3). Similar methods can be used to analyze FGF-5 expression in any sample from any organism.

To determine the FGF-5 and β-actin copy number, real-time PCR was used. Total RNA of normal adult human tissues were purchased from Clontech Laboratories (Palo Alto, Calif.). Total RNA from tumor cell lines were prepared using the RNeasy mini kit according to the manufacturer's instructions (Qiagen, Valencia, Calif.). First strand cDNA was synthesized using the Superscript Preamplification System (Life Technologies, Rockville, Md.) utilizing 5 μg of total RNA from either normal tissue or tumor cell lines. For the analysis, 2 μl out of 20 μl of the first strand cDNA was used. Two forward primers (FGF-F1 5′-CTTCTTCAGCCACCTGATCCTC (SEQ ID NO: 20) and FGF-F2 5′-TGCAGAGTGGGCATCGGTTTC (SEQ ID NO: 21)) were planned in exon 1 and two reverse primers (FGF-R15′-TATGCTCAATGCAGAGGTAC (SEQ ID NO: 22) and FGF-R2 5′-CGTAGTCCCTGTTATTTAAC (SEQ ID NO: 23)) were planned in exon 3.

Using the F1 and R1 pair and Taq polymerase (Life Technologies, Rockville, Md.), the first PCR reaction was performed at 94° C. for one minute followed by 16 cycles of 94° C. for 30 seconds, 61° C. for 45 seconds, and 72° C. for 60 seconds. As a template for the second nested PCR reaction, 2 μl out of 50 μl of the first PCR reaction was used with the primer pair of F2 and R2 using the same PCR program. The PCR product (10 μl) was subjected to agarose gel analysis. As a control, the expression level of β-actin was also measured.

To measure IFN-γ concentration, tumor cells (5×10⁴) were plated in flat bottom 96 well plates and 2×10⁴ Clone 2 CTL were added. After incubating for 20 hours, the supernatants were assayed for IFN-γ concentration by ELISA as described above in EXAMPLE 2.

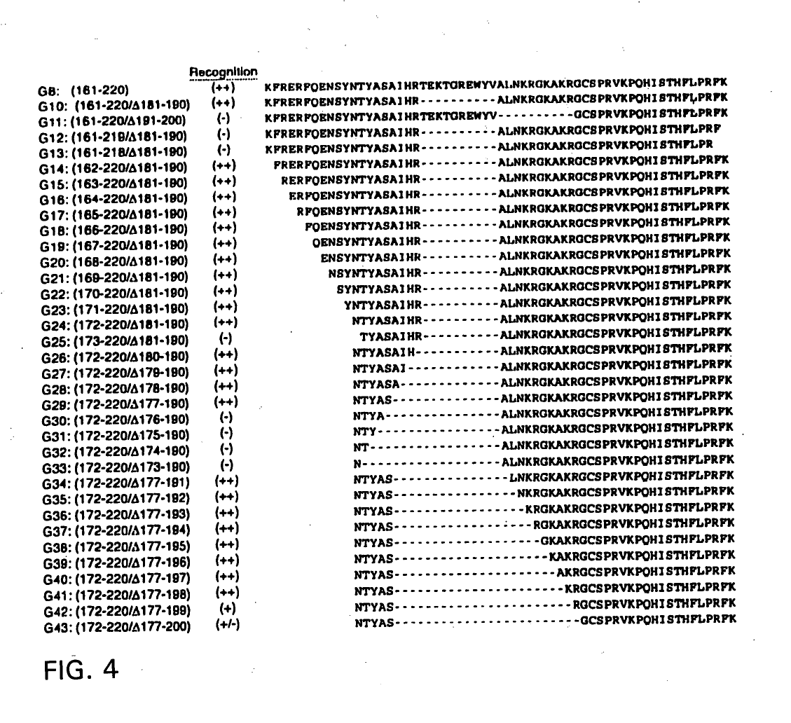

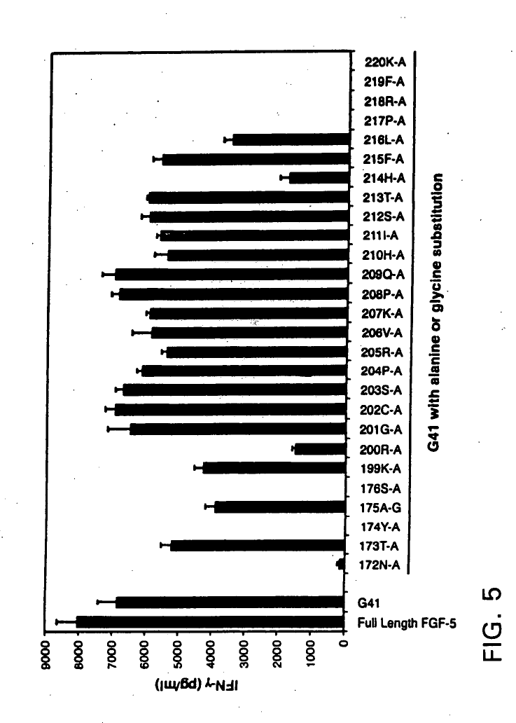

FGF-5 copy number was normalized to β-actin copy number in each cell line and the FGF-5 copy number/10⁵ β-actin copy number plotted (filled bars in FIGS. 4 and 5). In contrast to autologous RCC cell lines (for example 1764 RCC, FIG. 5) which showed strong FGF-5 expression, in the normal tissues analyzed, FGF-5 expression was below the level of detection. It was observed that in normal tissues, FGF-5 was only detectable in brain and kidney. However, the FGF-5 copy number in these tissues (about 20 FGF-5 copies/10⁵ β-actin copies) was lower than the calculated recognition threshold for CTL clone 2. No detectable FGF-5 expression was observed in normal tissues using Northern blotting.

In contrast, six of 10 RCC, two of three prostate carcinomas (PC3 and TSU-PR1) and 1 of 2 breast cancers (MDA231) showed significant recognition by CTL clone 2 (as judged by IFN-γ>50 pg/ml, and at least twice that generated against an autologous EBVB control). In addition, these carcinomas showed a higher FGF-5 copy number compared with normal tissues, for example at least 50%, at least 75%, at least 500% or even at least 1000% greater FGF-5 copy number. None of eight malignant melanomas (526, 586, 624.38, 1479, 501, 888, 1088, and 1199 mel), three lung cancers (SKGT-2, 4, and 5), one esophageal carcinoma (HCE-4), and two colon carcinomas (SW480 and CY13) were recognized. The recognition by CTL clone 2 highly correlated with FGF-5 copy number (p<0.0001). Marginally recognized cells such as 1570 RCC and TSU-PR1 indicates that the FGF-5 expression threshold for recognition by CTL clone 2 was 50-100 FGF-5 mRNA copies/10⁵ β actin copies. Therefore, cells having at least 75 FGF-5/10⁵ β-actin copies, for example at least 100 FGF-5/10⁵ β-actin copies, for example at least 500 FGF-5/10⁵ β-actin copies, for example at least 1000 FGF-5/10⁵-actin copies, are expected to generate an immune response.

### EXAMPLE 5

### Identification of an HLA-A3-Restricted Epitope

This example describes methods used to further identify the HLA-A3-restricted epitope recognized by Clone 2. Truncated fragments of the FGF-5 gene (nucleic acid cDNA sequence, Genbank Accession No: M37825 (SEQ ID NO: 3); amino acid sequence, Genbank Accession No: AAB06463 (SEQ ID NO: 4)) were cloned into the expression vector pME18S and tested by transfection into HLA-A3 expressing COS7 and recognition by Clone 2 assessed as follows.

Briefly, the reactivity of Clone 2 CTL was assessed by culturing COS cells transfected with the FGF-5 sequences shown in FIG. 4 (150 ng plasmid was used to transfect 5×10⁴ COS-A3 cells/well using Lipofectamine) in 96-well plates. After an overnight culture, 2×10⁴ cells/well of Clone 2 CTL were added. After incubating for 20-24 hours, supernatants were harvested and IFN-γ concentration was analyzed by ELISA (Endogen, Woburn, Mass.), where IFN-γ concentration is considered proportional to CTL activation. ELISA plates (96 well flat bottom, Costar, NY) were coated with anti-human IFN-γ monoclonal antibody (2G1, ENDOGEN, MA) at 1 μg/ml, 100 μl/well overnight. After washing, plates were blocked with PBS-5% FBS (fetal bovine serum) for one hour (200 μl/well), the samples added (50 μl/well) and incubated for 90 minutes. Subsequently, the plates were washed and biotin-labeled anti-human IFN-γ monoclonal antibody (B133.5, ENDOGEN, MA) was added at 0.5 μg/ml, 50 μl/well, and incubated for one hour. The plates were subsequently washed and 2000-times diluted HRP-streptavidin conjugate (Zymed Laboratories, CA) was added and incubated for 30 minutes. Plates were washed and DAKO TMB One-Step Substrate System (DAKO Corporation, CA) was added (100 μl/well). The coloration reaction was stopped by adding 0.18 M sulfuric acid (100 μl/well) and the optical density at 450 nm wave-length was measured. Recombinant human IFN-γ (Endogen, MA) was used as a standard.

Confirming the results shown in FIG. 3 (EXAMPLE 3), the shortest stimulatory fragment recognized encoded 60 amino acids (SEQ ID NO: 19), indicating that the A3 epitope resided between amino acids 161 and 220 of FGF-5 (Genbank Accession No: AAB06463). Further truncation of 36 nucleotides from the 5′ end or 24 bases from 3′ end of this minimal coding sequence obviated recognition by Clone 2.

HLA Class I binding peptides are typically 8-10 amino acids in length. Therefore, stepwise internal deletions were made within the mini-gene encoding the 60 amino acid fragment. Internal deletions of 30 nucleotides at a time demonstrated that removal of FGF-5 amino acids 181-190 (but not 191-200) resulted in a sequence that was recognized when transfected as a gene into an HLA-A3 target or when pulsed as a synthetic peptide on HLA-A3 EBV-B cells. Transfection and reactivity were assessed as described above. Pulsing of synthetic peptide involved co-incubation of EBV-B cells or dendritic cells (HLA-A3⁺ as well as HLA-A3⁻ controls) with high levels of various synthetic peptides from FGF-5 (at 1 nM-10 uM) in medium for 30-180 minutes. These cells were then either assayed or washed and assayed by the addition of Clone 2 T-cells for 24 hours and assay of supernatant IFN-γ by ELISA. Stepwise internal deletions were made, starting with a recognized mini-gene to identify residues important for epitope recognition.

As shown in FIG. 4, plasmid G41, encoding FGF-5 amino acids 172-176 and 199-220, was the smallest construct that was recognized as strongly as full-length FGF-5. However, some of the other deletion mutants retained immunogenic activity, just not a strongly as full-length FGF-5.

Subsequently, mini-genes with single alanine substitutions at each of the 27 codons within G41 (FIG. 5) were made to demonstrate the contribution of each amino acid to recognition. Point mutations were introduced by Quick Change Site-Directed Mutagenesis kit from Stratagene following the manufacture's protocol.

As shown in FIG. 5, important residues lay at the two termini of the peptide at positions 172 (N₁₇₂), 174 (Y₁₇₄), 176 (S₁₇₆), and 217-220 (P₂₁₇, R₂₁₈, F₂₁₉, and K₂₂₀) (with potential lesser contributions from amino acids 200 and 214). Peptides with Tyr at position 172 (position 3 of the mini-gene) and Phe/Lys at positions 219 and 220 (9/10 of the mini-gene) were favored for binding of HLA-A3 molecules.

Fusion peptides encompassing these two minimal terminal fragments were synthesized and tested for recognition by Clone 2 as follows. EBV-B cells were co-incubated with listed peptide at 10 μM, for 30 minutes at room temperature. After the incubation, EBV-B cells were washed and CTL were added. IFN-γ in the supernatant was measured 20-24 hours later by ELISA. Peptide pulsing was done in RPMI medium 1640 (RPMI) and the co-culture was done in RPMI with 10% fetal bovine serum (FBS). Peptides were synthesized either on the Pioneer Peptide Synthesizer (PE Biosystem) or on the AMS 222 multiple peptide synthesizer (Gilson) using standard F-moc chemistry. The molecular weights of peptides were verified by mass spectrometry (Bio-Synthesis Inc.).

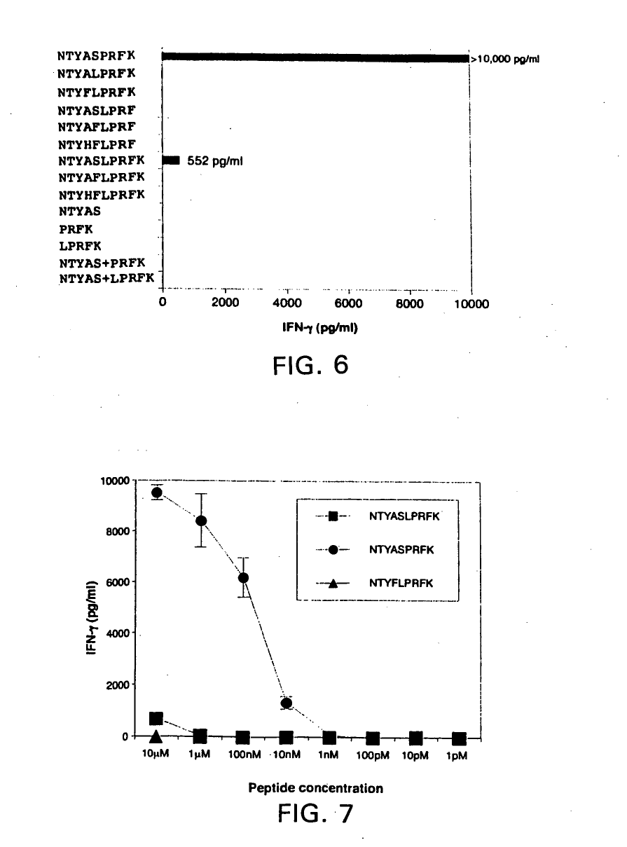

As shown in FIG. 6, two peptides, a 9-mer derived from 5 N-terminal amino acids and 4 C-terminal amino acids (of 172-176 plus 217-220; SEQ ID NO: 26) is strongly recognized, stimulating C2 cells at a concentration of 10 nM. The only other stimulatory peptide was the 10-mer NTYASLPRFK (172-176 plus 216-220; SEQ ID NO: 27), which demonstrated 1000-fold lower activity than SEQ ID NO: 1 (FIG. 6). Covalent fusion between NTYAS (amino acids 1-5 of SEQ ID NO: 26) and PRFK (amino acids 6-9 of SEQ ID NO: 26) provided a much better result than incubating APCs with even high concentrations of these peptides separately (FIG. 7, NTYAS+PRFK)

These results demonstrate that FGF-5 epitope generation involved a covalent intramolecular event involving the two terminal ends of the 172-220 peptide from FGF-5 generating the neo-epitope recognized. The determination that Clone 2 recognized 60% of RCC lines, as well as HLA-A3+FGF-5-transfected tumor lines, demonstrated that this protein-splicing event was common in FGF-5-expressing tumors. Titration of the 9-mer (SEQ ID NO: 26) showed it to be recognized at the nanomolar level while micromolar amounts of the 10-mer (SEQ ID NO: 27) were needed for stimulation of Clone 2. Therefore, the 9-mer neo-peptide (SEQ ID NO: 26) was processed and presented to T-cells in the context of HLA-A3 on FGF-5-expressing renal cancers.

### EXAMPLE 6

### Alanine Scan of FGF-5 HL HLA-A3 Epitope

An alanine scan was performed on the 9-mer (SEQ ID NO: 26), using standard methods, to determine which amino acids could tolerate an alanine substitution, without significant loss in immunoreactivity.

Position 173 of the peptide tolerates alanine substitution, and there is only partial reduction in activity with alanine substitutions at positions 172 (N₁₇₂), 176 (S₁₇₆) and 217 (P₂₁₇) (see Table 1). Therefore, epitopes having one or more amino acid substitutions at positions 172 (SEQ ID NO: 29), 173 (SEQ ID NO: 28), 176 (SEQ ID NO: 30) or 217 (SEQ ID NO: 31) are encompassed by this disclosure, as are other sequences with amino acid substitutions.

TABLE 1
Results of Alanine Scan of SEQ ID NO: 26.

| | | |
|---|---|---|
| | Resulting | |
| Alanine Substitution | Sequence | SEQ ID NO |

| | | |
|---|---|---|
| 9-mer with no substitution | NTYASPRFK | SEQ ID NO: 26 |
| (amino acids 172-176 and | | |
| 217-220 of FGF-5) | | |
| T173A | NAYASPRFK | SEQ ID NO: 28 |
| N172A | ATYASPRFK | SEQ ID NO: 29 |
| S176A | NTYAAPRFK | SEQ ID NO: 30 |
| P217A | NTYASARFK | SEQ ID NO: 31 |

### EXAMPLE 7

### HLA-A3 Epitope is Generated by Post-Translational Splicing

This example describes the methods used to demonstrate the mechanism that generates the 9-mer fusion peptide (SEQ ID NO: 26) from the FGF-5 gene.

To eliminate the possibility of ribosome skipping, three plasmids with termination codons inserted between the two determinant-encoding fragments were generated using standard molecular biology methods, and the ability of these sequences to activate Clone 2 determined as described above. No plasmids sensitized cells for Clone 2 recognition following transfection into COS-A3 APCs. This result also refutes epitope generation by aberrant RNA splicing because it appears that the intervening sequence between the two determinant-encoding fragments is transcribed and translated, indicating a post-translational mechanism.

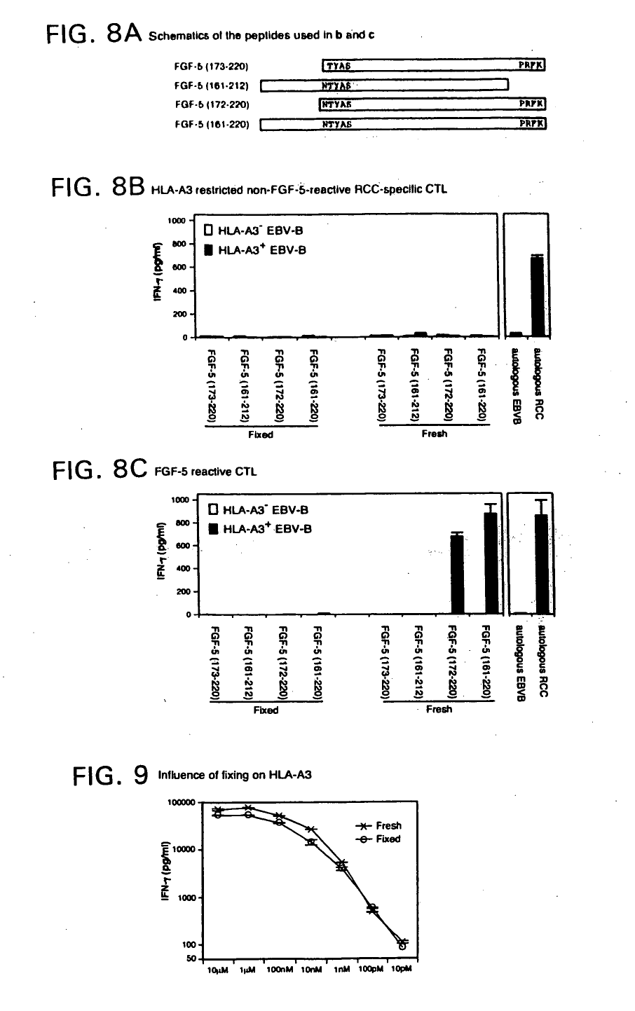

To demonstrate that the HLA-A3 epitope shown in SEQ ID NO: 26 is generated by post-translational protein splicing, the processing and recognition of synthetic peptides from FGF-5 (see FIG. 8A) pulsed onto HLA-A3⁺ or HLA-A3⁻ EBV transformed B (EBV-B) cells was determined as follows. EBV-B cells were co-incubated with 10 μM of the peptide for 3 hours at 37° C. After the incubation, EBV-B cells were washed and CTL were added. IFN-γ in the supernatant was measured 20-24 hours later by ELISA. Peptide pulsing was done in RPMI and the co-culture was done in RPMI with 10% FBS. Where indicated, EBV-B cells were fixed in 1% formaldehyde in PBS, for 10 minutes on ice, and washed in PBS. Peptides were synthesized and molecular weights verified as described in Example 5.

As shown in FIGS. 8B and 8C, high concentrations of a peptide corresponding to FGF-5 172-220 starting with the 5-mer (172-176, NTYAS; amino acids 1-5 of SEQ ID NO: 26) and ending with the 4-mer (217-220, PRFK; amino acids 6-9 of SEQ ID NO: 26) activated Clone 2 (but not another RCC-specific CTL clone) in a HLA-A3 restricted manner. Recognition was lost by deletion of residue 172 or residues 213-220 and was not affected by the addition of residues 161-171.

Presentation of the long peptide (SEQ ID NO: 38) was abrogated by mild aldehyde fixation of APCs, a process that did not affect the presentation of the 9-mer (SEQ ID NO: 26) (FIG. 9). Therefore, the results shown in FIGS. 8B and 8C are not the result of contamination of the 49-mer peptide (SEQ ID NO: 38) with 9-mer fusion peptide (SEQ ID NO: 26).

To confirm this observation, synthetic or acid-stripped 9-mer (SEQ ID NO: 26) and 49-mer (172-220; SEQ ID NO: 38) peptides were fractionated by HPLC and the capacity of fractions to activate Clone 2 determined as follows. COS-A3 and COS-A3/FGF-5 were prepared by retrovirally transducing COS-7 cells. Each cell line was cultured in T175 flasks and the peptides were stripped by adding 7.5 ml/flask of citrate buffer (0.13M citric acid, 0.056M sodium phosphate dibasic, pH 3.1) for 90 seconds. Peptide solutions (from ˜4×10⁹ cells, ˜1,200 ml each) were prepared and were concentrated by Sep-pak Plus C₁₈ column (Waters). After lyophilization and resolubilization into 200 μl of 5% acetonitrile 0.05% (v/v) TFA, peptides were fractionated using a C₁₈ HPLC column (218TP54, Grace Vydak) between 5% acetonitrile with 0.05% (v/v) TFA and 40% acetonitrile with 0.05% (v/v) TFA, linear gradient (1% and 1 ml/min). Fractions (1 ml each) were collected, lyophilized, reconstituted in 100 μl of RPMI and added to EBV-B. After 3 hours incubation, CTL in 100 μl of RPMI with 20% FBS were added and IFN-γ was measured at 20 hours. Synthetic peptides (9-mer 20 μg and 49-mer 100 μg) were HPLC fractionated and assayed in the same way as above. All the 9-mer fractions were diluted 100 times before the assay.

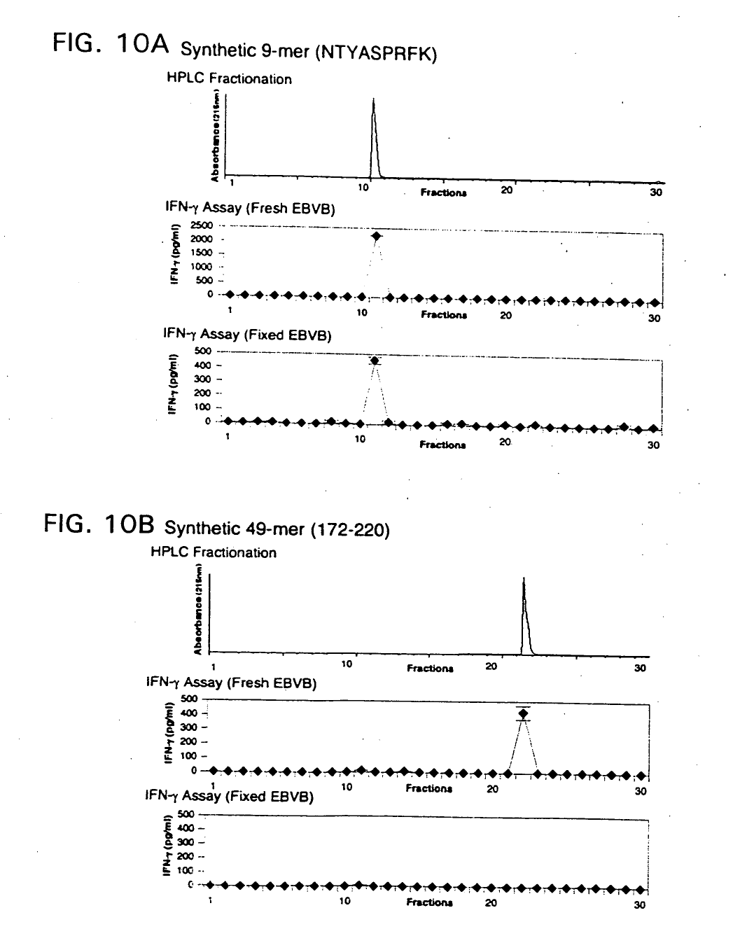

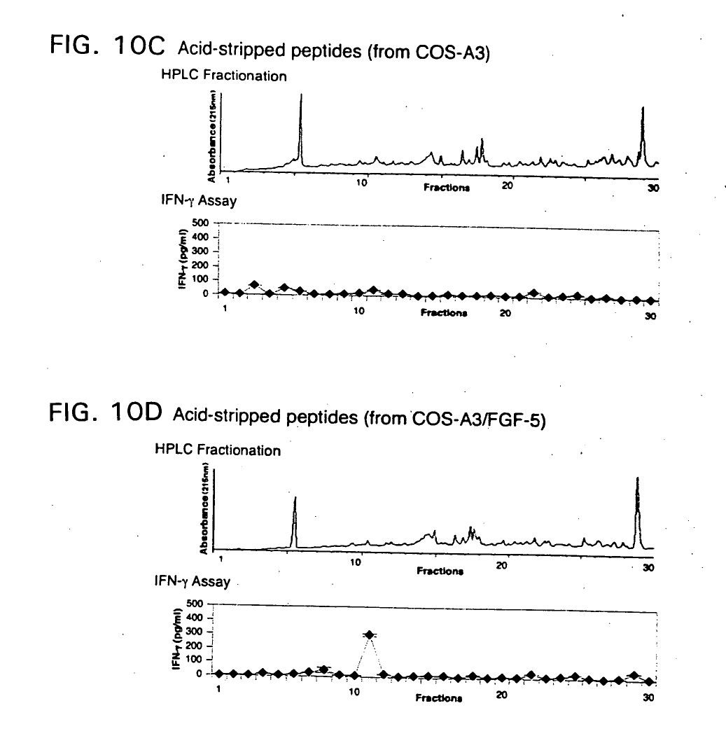

As shown in FIGS. 10A and 10B, the antigenic activities of the synthetic 9-mer (SEQ ID NO: 26) and 49-mer (SEQ ID NO: 38) eluted in fractions 11 and 22, respectively. These fractions contained the expected peptides as determined by mass-spectrometry. The 9-mer eluting in fraction in 11 was active using fresh or fixed APCs, while the 49-mer eluting in fraction 22 was active only using live cells.

HPLC also demonstrated that the 9-mer (SEQ ID NO: 26) represents the naturally processed peptide from FGF-5. As shown in FIG. 10D, antigenic activity of acid-stripped peptides from COS-A3 cells expressing FGF-5 was exclusively recovered from fraction 11. By contrast, none of the fractions from non-FGF-5 expressing cells demonstrated significant antigenic activity (FIC. 10C). These results indicate that the naturally processed FGF-5 determinant is the covalently-linked 9-mer (SEQ ID NO: 26) and not the 49-mer (SEQ ID NO: 38) nor the non-covalently bonded peptides NTYAS (amino acids 1-5 SEQ ID NO: 26) and PRFK (amino acids 6-9 SEQ ID NO: 26), which should elute in fractions 28 and 5 respectively.

Given the unusual origin of the 9-mer determinant (SEQ ID NO: 26), the antigen processing pathway utilized to generate it from full length FGF-5 was determined. An autologous RCC cell line that expresses melanoma antigen gp100, HLA-A2, A3, and DRβ1*0401 was generated by retroviral vector-mediated transduction of gp100, HLA-A2, and class II transactivator CIITA genes (CIITA), (the tumor is genotypically HLA-DRβ1*0401). The result is to create a tumor target capable of presenting not only its autologous antigens to Clone 2, but also class I and II-restricted determinants from gp100 to control T-cell clones which recognize this melanoma antigen (class II gp100 recognition is restricted by DRβ1*0401, naturally present on the RCC). Transduced RCC cells were treated with the citric acid buffer (pH 3.1) for 90 seconds, washed, then cultured in 10 μM clasto-Lactacystin β-Lactone (Calbiochem) for 3 hours. After washing, T-cells were added and IFN-γ was measured at 20 hours using the methods described in Example 5. TAP-1 dependency was determined by infecting the RCC line with either an adenovirus encoding GFP or TAP-1 inhibitor ICP47 (MOI=100, 2 hours). After washing, overnight culture and the treatment with the citric acid buffer as above, T-cells were added, and IFN-γ measured at 20 hours. A CD8⁺ T cell clone that recognizes the gp100₂₀₉₋₂₁₇ determinant was used as a positive control for inhibitor effectiveness and a CD4⁺ T cell clone that recognizes gp100₄₄₋₅₉ determinant was used as a control to eliminate non-specific effects of inhibitors on general antigen presentation capacity.

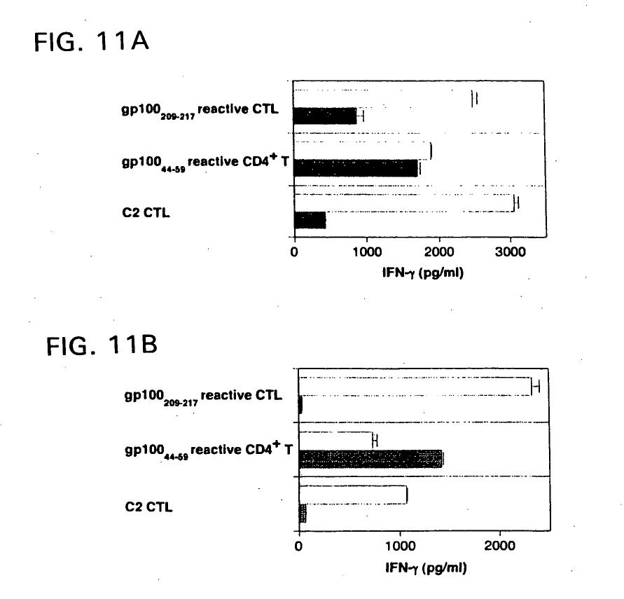

As shown in FIGS. 11A and 11B, presentation of both MHC class I-restricted peptides was blocked by the proteasome inhibitor clasto-Lactacystin β-lactone or by expressing ICP47 to inhibit TAP-mediated cytosol to ER peptide transport. MHC class II-restricted gp100 recognition was unaffected.

Therefore, it appears that the NTYASPRFK FGF-5 HLA-A3 peptide (SEQ ID NO: 26) is generated by protein splicing from longer biosynthetic or synthetic precursors. The splicing observed may likely occur via reverse proteolysis, as described for concanavalin A (Carrington et al. 1985. Nature 313: 64-7; Min and Jones. 1994. Nat. Struct. Biol. 1:502-4). The proteasome and TAP-dependence of antigen presentation and the successful splicing of plasmid encoded trunctated FGF-5 based-polypeptides lacking leader sequences indicate that splicing occurs in cytosol. In the presence of the leader sequence on FGF-5, delivery of FGF-5 to the cytosol for splicing may require the endoplasmic reticulum associated degradation pathway.

These results also demonstrate that the immune system monitors non-contiguous peptide sequences generated post-translationally. This capability represents an enormous increase in the ability of CTLs to recognize self and foreign proteins. While this could enhance immune surveillance, it does complicate the task of identifying peptide ligands recognized by tumor- and pathogen-specific CTL.

### EXAMPLE 8

### Identification of an HLA-A2-Restricted Epitope

An HLA-A2-restricted epitope of FGF-5 was identified using the following methods.

Peripheral blood lymphocytes (PBL) from HLA-A2 subjects were stimulated with FGF-5 peptides. If peptide reactivity was generated in vitro, these peptide-reactive T-cell cultures were tested for recognition of FGF-5 expressing, HLA-A2 positive tumors. Synthetic peptide preparations were used for approximately a dozen candidate peptides. Using one preparation, 5194 (synthesized by Macromolecular Resources, Ft. Collins, Colo.), that was reportedly a peptide from the N-terminal leader sequence of FGF-5, and repetitively stimulating PBL from an HLA-A2 positive RCC subject using the synthetic peptide preparation containing FGF-5 sequences, a culture which recognized the stimulating peptide preparation pulsed onto A2+cells (but not a control peptide) and recognized an FGF-5 expressing HLA-A2+RCC line (but not the same tumor without HLA-A2) was obtained. This was cloned by limiting dilution and six clones with the same reactivity were obtained. These clones recognized peptide, FGF-5⁺/HLA-A2⁺ tumor as well as HLA-A2⁺ COS cells transfected with the gene for FGF-5, but not the control vector, confirming that the peptide epitope was naturally processed from full length FGF-5 and presented on HLA-A2 (FIG. 12).

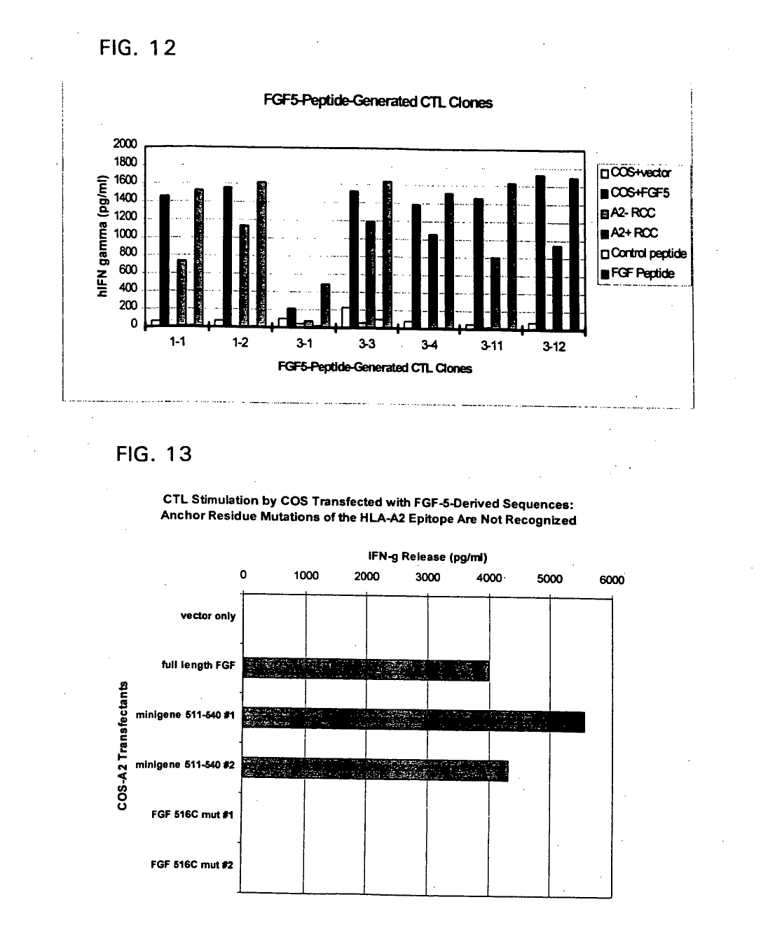

The actual peptide recognized was determined by testing truncations of the FGF-5 gene transfected into HLA-A2⁺ cells. A2⁺ target cells transfected with the full-length FGF-5 gene were recognized. Truncations of the FGF-5 gene localized the epitope to a region overlapping nucleotides 467-541 of FGF-5 (Genbank Accession No: M37825). A candidate A2-binding 10-mer peptide was present within this region (encoded by nucleotides 511-540 of Genbank Accession No: M37825, corresponding to amino acids 117-126 of Genbank Accession No: AAB06463) and a purified synthetic preparation of this peptide, MLSVLEIFAV (SEQ ID NO: 32), was well recognized by tumor-reactive CTL clones when pulsed onto T2 cells in nanomolar amounts (FIG. 13).

To further demonstrate that MLSVLEIFAV (SEQ ID NO: 32) is an FGF-5 HLA-A2 epitope, site-directed mutagenesis was used to convert A to C at nucleotide 516 (of Genbank Accession No: M37825; SEQ ID NO: 3) which resulted in a substitution of phenylalanine for the native leucine at the HLA-binding anchor residue (L118F) at position two of SEQ ID NO: 32. This modified gene product (MFSVLEIFAV; SEQ ID NO: 33) was no longer recognized by the CTL clones when transfected into HLA-A2⁺ target cells, demonstrating that Leu 118 is important for recognition by tumor-reactive T-cell clones. HLA-A2-binding substitutions can be made at possible anchor residues, such as amino acid Leu 118, and amino acid Val 126, to improve immunogenicity of this epitope.

### EXAMPLE 9

### Overview of Methods of Using FGF-5 Epitopes

Having demonstrated that tumor-reactive T-cells generated from subjects with RCC can recognize naturally presented FGF-5 in either the context of HLA-A2 or HLA-A3 via the minimal determinants MLSVLEIFAV (SEQ ID NO: 32) or NTYASPRFK (SEQ ID NO: 26), respectively, such peptides (as well as variants, fragments, and fusions thereof) can be used as an immunotherapy, for example in subjects with an FGF-5 expressing or overexpressing tumor, such as an adenocarcinoma. The present disclosure provides methods for immune stimulation (for example vaccination) using these peptides (or corresponding nucleic acids, or T cells stimulated with these peptides) to enhance the number of FGF-5-reactive CTL precursors in subjects with an FGF-5 expressing or overexpressing tumor, such as RCC, or affect the anticipated response rate from high-dose IL-2.

Although some of the examples herein describe particular methods of administration, and particular FGF-5 HLA-A3 and -A2 epitope sequences (such as SEQ ID NO: 26 or 32), one skilled in the art will understand that other FGF-5 HLA-A3 or -A2 epitopes can be administered using other methods (for example see Example 17). In addition, variants, fragments, or fusions of SEQ ID NO: 26 or 32 (for example see Examples 18, 19, and 22), for example sequences that include SEQ ID NOS: 27-31, 34, and 39, can be administered. In addition to administering only an FGF-5 HLA-A3 or -A2 epitope to a subject, the present disclosure includes administration of a combination of an FGF-5 HLA-A3 epitope and an FGF-5 HLA-A2 eptiope to a subject, such as to a subject who is both HLA-A2⁺ and HLA-A3⁺ (for example see Example 16).

In one example, the method includes haplotying the subject, if this information is not already known. This permits administration of the appropriate FGF-5 HLA antigen.

The method can also include determining if the tumor expresses or overexpresses FGF-5 (for example see Example 15). The disclosed immunogenic compositions can be administered to subjects having an FGF-5 expressing or overexpressing tumor.

The method can include administering a therapeutically effective amount of one or more FGF-5 HLA-A3 or -A2 epitope protein or nucleic acid sequences, or T cells stimulated with the epitope. In particular examples, the subject is administered at least one 0.1-10 mg dose of FGF-5 HLA-A3 or -A2 epitope peptide, in the presence of a pharmaceutically acceptable carrier, such as an adjuvant or an agent that potentiates the immune response (for example an adjuvant or cytokine, such as interleukin-12). The initial dose can be followed by booster doses, following immunization protocols standard in the art. For example, 2-15 or 2-11 additional doses can be administered. The methods disclosed herein can also be combined with other anti-neoplastic treatments, such as radiotherapy or administration of anti-neoplastic drugs or biologics (such as IL-2).

In one example, the method includes administering a therapeutically effective amount of autologous CTLs, specific to an FGF-5 HLA-A3 or -A2 epitope, to a subject having an FGF-5 expressing or overexpressing tumor. Autologous PBMCs such as lymphocytes, or cells of dendritic origin, can be used. Generally, a sample of cells obtained from a subject, such as blood cells, are contacted with a cell presenting the FGF-5 HLA-A3 or -A2 epitope and capable of provoking CTLs to proliferate. In addition to the target cells listed above, the target cell can be a transfectant, such as a COS cell. Cells present the desired HLA complex on their surface and, when combined with a CTL of interest, stimulate proliferation of the CTL of interest. COS cells. Specific production of a CTL clone is described herein. The clonally expanded autologous CTLs are administered to the subject. Another method that can be used is adoptive transfer. Cells presenting the FGF-5 HLA-A3 or -A2 epitope are combined with CTLs, leading to proliferation of the specific CTLs. The proliferated CTLs are administered to a subject having from an FGF-5 expressing tumor, such as RCC, carcinoma of the breast, prostate, bladder or pancreas. The CTLs then lyse the FGF-5 expressing tumor cells.

CTLs can also be stimulated in vivo. One approach is the use of non-proliferative cells expressing the complex of FGF-5 and an FGF-5 HLA-A3 or -A2 epitope, to stimulate an immune response. The cells used can be those that normally express the complex, such as irradiated tumor cells or cells transfected with a sequence that encodes for an FGF-5 HLA-A3 or -A2 epitope, such as a vector. The cells which result present the complex of interest and are recognized by autologous CTLs, which then proliferate.

### EXAMPLE 10

### Administration of FGF-5 Immunogenic Peptides

This example describes methods of administering immunogenic FGF-5 HLA-A2 or -A3 epitopes (SEQ ID NO: 26 and 32, respectively) to a subject to determine response rates and toxicity of the peptides. The peptides are administered to HLA-appropriate subjects with RCC and the effect of such administration on the response rate in the presence or absence of high-dose IL-2 determined.

Subjects with advanced clear cell renal cancer expressing FGF-5, are administered an HLA-appropriate peptide vaccination (SEQ ID NO: 32 for HLA-A2⁺ subjects or SEQ ID NO: 26 for HLA-A3⁺ subjects) emulsified in Montanide ISA-51 adjuvant (ISA). Subjects are divided into cohorts with measurable metastatic disease (cohorts A and B) or high-risk loco-regional disease (cohort C). Subjects with measurable metastatic disease are separated into those that receive immediate IL-2 therapy (cohort B) or those who do not (cohort A) as determined by tumor burden, tempo of disease or other co-morbidities.

Subjects in cohort A begin receiving vaccination with HLA-appropriate peptide emulsified in Montanide ISA-51 adjuvant (a water-in-oil emulsion) (ISA) every 3 weeks. This is continued for up to a year, or until tumor progression (see Example 14) is documented. If tumor progression is documented, subject's eligible for IL-2 who have not yet received it can have high-dose intravenous bolus IL-2 added to their peptide vaccination regimen. Throughout, all IL-2 is administered at 720,000 IU/kg/dose every 8 hours by intravenous bolus to the maximum tolerated number of doses (which constitutes one cycle of therapy). Two cycles, separated by 10-14 days, is administered during every two month period, with further treatment dependent on interval tumor assessment. For subjects in Cohort A crossing over to vaccination plus IL-2 therapy, a single peptide+ISA vaccination is administered the day prior to starting an IL-2 cycle (instead of every three weeks, to accommodate the standardized IL-2 regimen).

Subjects in Cohort B who receive immediate IL-2 therapy begin with the same standard high-dose bolus IL-2 therapy in two cycles within every two month period, with each cycle preceded by a peptide+ISA administration the day prior to starting each IL-2 cycle. Because subjects with metastatic RCC are likely to progress within a few months (unless vaccination alone has an early and dramatic effect) and because starting vaccination and peptide simultaneously may blunt the ability to measure immune response in the blood, it is desirable to have a subject population who can safely undergo a more prolonged period of vaccination without receiving IL-2. However, methods of administration also include concurrent administration of IL-2.

Subjects with advanced loco-regional disease have a poor prognosis and no adjuvant therapy options of efficacy. Therefore, Cohort C includes subjects who have undergone resection of either T3/T4 or N1/N2 primary tumors (Stage III disease) within six months of beginning therapy. These subjects undergo the same HLA-appropriate vaccination with peptide and ISA every three weeks and continue for up to one year or until disease relapse is documented. At the time of relapse, eligible subjects in Cohort C receive standard treatment with high-dose bolus IL-2 and continuing peptide vaccination.

Within each cohort, there are subgroups of subjects who are HLA-A2 or HLA-A3 (subjects who have both alleles are treated with an HLA-A3 peptide, SEQ ID NO: 26, or can be administered both peptides as described in Example 16). HLA-A2 subjects are injected subcutaneously with 1 mg of MLSVLEIFAV (SEQ ID NO: 32) emulsified in ISA and HLA-A3 subjects receive 1 mg of NTYASPRFK (SEQ ID NO: 26) in ISA. The 1 mg dose of peptide was selected based on immunization with peptides derived from the MART-1 and gp100 melanoma antigens, where no significant dose response relationship was observed over 0.1 mg to 10 mg of peptide (Cormier et al., 1997. Cancer J. Sci. Am. 3:37-44; Salgaller et al., 1996 Cancer Res. 56:4749-57).

TABLE 2
Examples of Treatment Methods (Each cohort has
subgroups for HLA-A2 and HLA-A3)

| Cohort A | Cohort B | Cohort C |
|---|---|---|
| Metastatic disease | Metastatic disease | Resected Stage |
| | | III disease |
| able to delay IL-2 | receives immediate IL-2 | |
| Starts vaccine alone once | Starts standard IL-2 with | Starts vaccine |
| every 3 wks | vaccine prior to each cycle | alone once |
| | | every 3 wks |
| If PD seen, and IL2- | If PD seen, patient taken | If relapse seen, |
| eligible, start standard IL- | off of protocol | without sur- |
| 2 with vaccine prior to | | gical option, |
| each cycle | | starts standard |
| | | IL-2 with |
| | | vaccine prior to |
| | | each cycle |
| Evaluations once every 6 | once every 2 months while | once every 3 |
| weeks on peptide and once | on IL-2; once every 3-6 | months while |
| every 3-6 months if stable | months if stable after | on peptide once |
| beyond 6 months; once | | every 2 months |
| every 2 months while on | | while on IL-2; |
| IL-2 | | once every 3-6 |
| | | months if |
| | | stable after |

Peptides

HLA-A2-restricted FGF-5 peptide: FGF-5: 117-126 (SEQ ID NO: 32) and HLA-A3-restricted FGF-5 peptide: FGF-5: 172-176+217-220 (SEQ ID NO: 26) were produced to GMP grade by solid phase synthesis techniques by Multiple Peptide Systems (San Diego, Calif.). The finished injectable dosage form is supplied as a 5 ml clear molded glass, siliconized vial containing 0.2 ml of a frozen sterile solution. Each mL contains 10 mg of each peptide and approximately 1.5 mg (0.1% v/v) of trifluoroacetate in DMSO. The vials are stored at −70° C. until use. The peptides are used within 3 hours after thawing.

The desired peptide is reconstituted and injected as an emulsion with ISA for injection in the anterior thigh deep subcutaneous tissue (two 1 ml injections) as follows. To prepare a 1 mg dose, add 1.8 ml of Montanide ISA-51 to a vial of the peptide (10 mg/ml, 0.2 ml/vial) and an equal volume (2 mL) of 0.9% sodium chloride injection, USP. The vial is vortexed for 12 minutes, and 2.0 ml is withdrawn for administration. The peptide vaccine emulsion is administered as two 1 mL injections (0.5 mg/mL) in the anterior thigh deep subcutaneous tissue, within 2 cm of each other. If the subject has previously undergone lymph node dissection, that extremity will not be used and an upper extremity can be used.

Montanide ISA-51 (Seppic, Inc.) will be used as the Montanide ISA-51 (Fairfield, N.J.). It is provided as an amber glass ampule containing 3 ml of a mineral oil solution based on mannide oleate. At the time of injection, peptide is mixed with the Montanide ISA-51 as described above. Montanide ISA-51 can be filtered wit a 5 micro filter prior to use.

Other adjuvants can be used, for example, Freund's complete adjuvant, B30-MDP, LA-15-PH, Montanide, saponin, aluminum hydroxide, alum, lipids, keyhole lympet protein, hemocyanin, a mycobacterial antigen, and combinations thereof.

Interleukin-2 Administration

In particular examples, IL-2 (Chiron Corp., Emeryville, Calif.) is administered at a dose of 720,000 IU/kg as an intravenous bolus over a 15 minute period every eight hours beginning on the day after immunization and continuing for up to 4 days (a maximum of 12 doses). Doses can be skipped depending on subject tolerance. Doses are skipped if subjects reach Grade III or IV toxicity due to IL-2 except for the reversible Grade III toxicities common to IL-2 such as diarrhea, nausea, vomiting, hypotension, skin changes, anorexia, mucositis, dysphagia, or constitutional symptoms and laboratory changes as detailed in Table 2. If this toxicity is easily reversed by supportive measures then additional doses can be given.

IL-2 is provided as a lyophilized powder. The vial is reconstituted with 1.2 ml of sterile water for injection, USP, and the resultant concentration is 18 million IU/ml. Since vials contain no preservative, reconstituted solution is used with 8 hours. Reconstituted IL-2 is further diluted with 50 ml of 5% human serum albumin (HSA). The HSA is added to the diluent prior to the addition of RIL-2. Dilutions of the reconstituted solution over a 1000-fold range (1 mg/ml to 1 mcg/ml) are acceptable in either glass bottles or polyvinyl chloride bags. Ideally, IL-2 is not mixed with saline-containing solutions.

### EXAMPLE 11

### Evaluation During Treatment: Measurement of IFN-γ Response

In a particular example, a subject's response to treatment is evaluated by a complete blood count, acute care, hepatic and mineral panels every three weeks. Subjects receiving IL-2 can obtain a CBC, acute care, hepatic and mineral panel evaluated every 1 to 2 days of treatment. Peptide immunizations can be administered as an outpatient, unless inpatient admission is indicated for treatment of vaccine-related side-effects or underlying disease management.

Subjects are monitored after each injection with vital signs twice within the first hour and are discharged from clinic at that time if stable. Biopsies of tumor tissue or lymph node can be performed, but are not required during the course of therapy. Apheresis is performed prior to the first and third immunizations and three weeks after the last immunization. The apheresis can include a 5-10 liter exchange to last 2-4 hours. If not possible, collection of 50-80 ml of peripheral blood can be substituted.

To measure an immune response in a subject following administration of the therapeutic peptides, the release of IFN-γ from PBMCs obtained from the subject before and after administration can be determined. For example, peripheral blood lymphocytes (PBL) are purified by centrifugation on a Ficoll cushion, then incubated in the presence of the FGF-5 HLA-A2 or -A3 immunogenic peptide (or a control peptide), to stimulate proliferation of FGF-5-specific CTL precursors, as previously described (Cormier et al., 1997. Cancer J. Sci. Am. 3:37-44; Salgaller et al., 1996 Cancer Res. 56:4749-57). The PBMCs are obtained before and after administration of the FGF-5 HLA-A2 and -A3 peptides, to determine if there is an increase in the specific release of gamma interferon following the vaccination. FGF-5-specific CTL can be determined using a cytokine release assay, or an ELISPOT assay using tumor, FGF-5-transfected or peptide-loaded target cells and compared to pre-treatment PBMC to determine immune response to vaccination, as previously described (Id.). In general, differences of 2 to 3 fold in these assays are indicative of true biologic differences. Repeated in vitro sensitizations can be performed on a weekly basis if needed to elicit differences between pre and post immunization samples. A positive assay is the release of 0.1 pg IFN-γ/mL/24 hours. Post immunization samples that are at least two-fold greater in value than pre-immunization samples are considered positive (that is, the subject has had an elicited or stimulated immune response to the epitope).

### EXAMPLE 12

### Evaluation Following Treatment

For subjects in cohort A on peptide vaccine alone, complete physical evaluation, CBC, acute care, hepatic and mineral panels and appropriate X-ray evaluations of all evaluable lesions are obtained every six weeks during the first six months of therapy and if stable, every 3-6 months thereafter. Other evaluations can be performed as indicated by symptoms or physical findings.

For cohorts A and B during peptide vaccine plus high-dose IL-2 therapy, complete physical evaluation, CBC, acute care, hepatic and mineral panels and appropriate X-ray evaluations of all evaluable lesions are obtained every two months while on IL-2, and every 3-6 months for stable subjects off therapy.

For cohort C, complete physical evaluation, CBC, acute care, hepatic and mineral panels are performed every three months for the first year and every six months thereafter. Surveillance CT of the chest, abdomen and pelvis are obtained with at least every other assessment and as indicated by symptoms or physical findings.

### EXAMPLE 13

### Additional Treatment

In particular examples, if subjects are stable or have a minor, mixed or partial response to treatment, they can be re-treated after re-evaluation with the same schedule and preparation of peptide that they previously received for up to a year of total therapy.

A mixed response is the shrinkage of some lesions but an increase in others. Subjects with mixed responses may only receive treatment for an additional 2-3 months without showing true disease stability or a bona fide minor or major response (i.e. no further progression). A maximum of two re-treatment cycles can be given following a complete response.

### EXAMPLE 14

### Evaluation of Lesions

All measurable lesions, up to a maximum of 10 lesions, representative of all involved organs are identified as target lesions and recorded and measured at baseline. Target lesions are selected on the basis of their size (lesions with the longest diameter) and their suitability for accurate repetitive measurements (either by imaging techniques or clinically). A sum of the longest diameter (LD) for all target lesions is calculated and reported as the baseline sum LD. The baseline sum LD is used as reference to further characterize the objective tumor response of the measurable dimension of the disease as outlined in Table 3.

TABLE 3
Measurement of Target Lesion Response.

| Complete | |
|---|---|
| Response (CR) | Disappearance of all target lesions |
| Partial Response | At least a 30% decrease in the sum of the longest |
| (PR) | diameter (LD) of target lesions taking as reference |
| | the baseline sum LD. |
| Progression (PD) | At least a 20% increase in the sum of LD of target |
| | lesions taking as reference the smallest sum LD |
| | recorded since the treatment started or the appearance |
| | of one or more new lesions. |
| Stable Disease | Neither sufficient shrinkage to qualify for PR nor |
| (SD) | sufficient increase to qualify for PD taking as |
| | references the smallest sum LD. |
| Mixed Response | A shrinkage of some lesions but an increase in others |

All other lesions (or sites of disease) are identified as non-target lesions and recorded at baseline. Measurements are not required, and these lesions are followed as “present” or “absent.”

TABLE 4
Measurement of Non-Target Lesion Response.

| | |
|---|---|
| Complete Response | Disappearance of all non-target lesions and |
| (CR): | normalization of tumor marker level. |
| Non-Complete Response: | Persistence of one or more non-target lesions |
| Progression (PD): | Appearance of one or more new lesions. |
| | Unequivocal progression of existing non-target |
| | lesions |

The best overall response is the best response recorded from the start of the treatment until disease progression/recurrence (taking as reference for progressive disease the smallest measurements recorded since the treatment started). The subject's best response assignment can depend on the achievement of both measurement and confirmation criteria as shown in Table 5.

TABLE 5
Evaluation of Overall Response.

| Target Lesions | Non-Target Lesions | New Lesions | Overall Response |
|---|---|---|---|
| CR | CR | No | CR |
| CR | Non-CR/Non-PD | No | PR |
| PR | Non-PD | No | PR |
| SD | Non-PD | No | SD |
| PD | Any | Yes or No | PD |
| Any | PD | Yes or No | PD |
| Any | Any | Yes | PD |

To be assigned a status of PR or CR, changes in tumor measurements are confirmed by repeat studies that are performed at least 4 weeks after the criteria for response are first met. In the case of SD, follow-up measurements met the SD criteria at least once after study entry at a minimum interval of 6-8 weeks.

The duration of overall response is measured from the time measurement criteria are met for CR/PR (whichever is first recorded) until the first date that recurrent or progressive disease is objectively documented (taking as reference for progressive disease the smallest measurements recorded since the treatment started). The duration of overall complete response is measured from the time measurement criteria are first met for CR until the first date that recurrent disease is objectively documented.

Duration of stable disease is measured from the start of the treatment until the criteria for progression are met, taking as reference the smallest measurements recorded since the treatment started.

### EXAMPLE 15

### Screening Tumors for FGF-5 Expression

This example describes methods to determine if a tumor in a subject expresses or over-expresses FGF-5, for example by detecting the presence of FGF-5 nucleic acid molecules or proteins in a sample obtained from the subject, such as a sample from the tumor. In one particular example disclosed herein, a tumor present in a subject is pre-screened to determine if the tumor expresses or over expresses FGF-5. Subjects having an FGF-5 expressing tumor can be selected for therapy with one or more FGF-5 immunogenic epitopes, such as those disclosed herein. Methods for determining expression of a particular nucleic acid molecule or protein are known in the art. Therefore, although particular examples are provided herein, the disclosure is not limited to these particular methods.

A “control” sample from the subject (or another subject), can be used to compare FGF-5 expression. For example, a positive control can include an amount of FGF-5 protein or nucleic acid molecule expected if a tumor expresses or over-expresses FGF-5. In contrast, a negative sample would include an amount of FGF-5 protein or nucleic acid molecule expected if the tumor does not expresses or does not over-expresses FGF-5. As disclosed in Example 4 above, FGF-5 was not expressed in detectable levels in any normal tissue tested.

Detection of FGF-5 Nucleic Acid Molecules

A tumor sample obtained from the subject that contains nucleic acids can be used to determine if the tumor has FGF-5 nucleic acid molecules.

One method that can be used to detect FGF-5 is by RT-PCR from a fine needle tumor biopsy. RNA is prepared from the tumor biopsy sample as follows. The fine needle aspirate (FNA) sample is centrifuged at approximately 500×g for 5 minutes (for a 15 ml conical tube) or at 8,000 RPM for 1 minute (for a 1.5 ml. microfuge tube). For the preparation of RNA from PBL, 10 ml of peripheral blood is centrifuged in a CPT tube at 2,400 RPM for 20 minutes. The lymphocyte layer is collected and at least 10 volumes of phosphate buffered saline (PBS) added and centrifuged at 800 RPM for 10 minutes (table-top centrifuge).

The resulting supernatant is removed, and if needed, cell pellets can be flash-frozen in liquid nitrogen and preserved at −70° C. for later processing. Total RNA is prepared from the cell pellet using RNeasy (Qiagen, Valencia, Calif.) following the manufacturer's protocol. When frozen samples are processed, samples are kept on dry-ice to avoid thawing before adding the buffer. Contaminating DNA is removed by the on-column DNase digestion y. Elution of RNA from the column is done using 30 μl RNase-free water. The concentration of RNA is determined by OD.

First strand cDNA synthesis is performed using SuperScript First-Strand Synthesis System for RT-PCR (Invitrogen, Carlsbad, Calif.) and 100 μg total RNA and oligo-(dT) primer. Each sample is evaluated using the 6 conditions outlined in Table 6:

TABLE 6
RT-PCR conditions.

| | | | | | Positive Control (total | | | | | | | |
|---|---|---|---|---|---|---|---|---|---|---|---|---|
| | Negative Control (total | | | | RNA from standard | | | | Patient Sample (total | | | |
| | RNA from patient PBL) | | | | RCC tumor line 1764) | | | | RNA from FNA) | | | |
| | No RT | | | | No RT | | | | No RT | | | |
| Component | control | | Sample | | control | | Sample | | control | | Sample | |
| total RNA | 100 | ng | 100 | ng | 100 | ng | 100 | ng | 100 | ng | 100 | ng |
| 10 mM dNTP | 1 | μl | 1 | μl | 1 | μl | 1 | μl | 1 | μl | 1 | μl |
| oligo(dT) | 1 | μl | 1 | μl | 1 | μl | 1 | μl | 1 | μl | 1 | μl |
| primer | | | | | | | | | | | | |
| (0.5 ug/ul) | | | | | | | | | | | | |
| DEPC water | to 10 | μl | to 10 | μl | to 10 | μl | to 10 | μl | to 10 | μl | to 10 | μl |

Each sample is incubated at 65° C. for 5 minutes, and then placed on ice for at least 1 minute. The following reaction mixture is prepared, adding each component in the indicated order.

| Component | Each Reaction |
|---|---|
| 10x RT buffer | 2 μl |
| 25 mM MgCl₂ | 4 μl |
| 0.1 M DTT | 2 μl |
| RNaseOUT ™ RNase inhibitor | 1 μl |

Reaction mixture (9 μl) is added to each RNA/primer mixture, mixed gently, and collected by brief centrifugation, and then incubated at 42° C. for 2 minutes. 1 μl (50 units) of SuperScrip™ RT is added to each tube, mix, and incubate at 42° C. for 50 min. Terminate the reactions at 70° C. for 15 minutes and chill on ice. The reaction is collected by brief centrifugation. Add 1 μl of RNase H to each tube and incubate for 20 min at 37° C. before proceeding to the PCR reaction.

The PCR reaction is performed with the following primers: β-actin forward primer 5′-ATTGGCAATGAGCGGTTCCGC-3′ (SEQ ID NO: 40) and reverse primer

| | |
|---|---|
| 5′-AGGTAGTTTCGTGGATGCCAC-3′; | (SEQ ID NO: 41) |
| FGF-5 forward primer | |
| 5′-AGTCAATGGATCCCACGAAG-3′ | (SEQ ID NO: 42) |
| and | |
| reverse primer | |
| 5′-CTTGAAAACGCTCCCTGAAC-3′. | (SEQ ID NO: 43) |

The PCR reaction mixture includes the following (per reaction): 2 μl cDNA; 5 μl 10×PCR buffer Mg(−) (200 mM Tris-HCl (pH 8.4), 500 mM KCl); 1 μl 10 mM dNTP mix; 1.5 μl 50 mM MgCl₂; 2.5 μl each forward and reverse primer (10 mM each); 0.25 μl Taq DNA polymerase; and 35.25 μl water. The PCR program is as follows: 96° C. for 120 sec; followed by 30 times of 96° C. for 30 sec, 55° C. for 30 sec, and 72° C. for 30 sec; followed by 72° C. for 300 sec and 4° C.

Agarose gel analysis is performed to detect FGF-5 PCR products. Samples undergo electrophoresis on 2% agarose gel with standard nucleic acid size markers. Predicted band sizes are 78 bp for β-actin and 179 bp for FGF-5. A positive sample is one that has β-actin detectable at 78 bp in PBL, tumor and RCC 1764, with the tumor and RCC standard displaying a 179 bp band, but no 179 bp band in the PBL sample (and no bands of either size in the absence of reverse transcriptase).

One skilled in the art will appreciate that other methods can be used to detect FGF-5 nucleic acids in a sample. For example, nucleic acids isolated from the sample can be incubated with an FGF-5 specific nucleic acid probe (that has a detectable label, such as ³²P or a fluorophore) under conditions that permit hybridization between the probe and an FGF-5 nucleic acid sequence, wherein presence of detectable label following the hybridization indicates that tumor is an FGF-5 expressing tumor. The absence of detectable hybridization indicates that the tumor does not express FGF-5. Binding between an FGF-5 sequences and a probe can be detected by any procedure known to one skilled in the art, including both functional and physical binding assays.

Detection of FGF-5 Proteins

Another method that can be used to determine if a tumor in a subject expresses FGF-5 is to determine if FGF-5 protein is present in the tumor, for example by using an immunoassay. A tumor sample obtained from the subject that contains proteins can be used to determine if the tumor expresses or overexpresses FGF-5 proteins. Typical methods involve contacting a biological sample of the subject containing cellular proteins with an FGF-5 specific-binding agent (such as an FGF-5 antibody) under conditions sufficient to form complexes between the binding agent and the FGF-5 protein present in the sample. The resulting complex is detected, and can be quantitated. The presence of detectable complex indicates that tumor is an FGF-5 expressing tumor, while the absence of detectable complex indicates that the tumor does not express FGF-5. An increase (such as an increase of at least 20%, or at least 50%) of the complexes relative to specific binding agent:FGF-5 protein complexes in a non-neoplastic cell indicates that the tumor expresses or overexpresses FGF-5.

In particular forms, these assays are performed with the FGF-5 specific binding agent immobilized on a support surface, such as in the wells of a microtiter plate or on a column. The biological sample is then introduced onto the support surface and allowed to interact with the specific binding agent so as to form complexes. Excess biological sample is removed by washing, and the complexes are detected with a reagent, such as a second anti-FGF-5 protein antibody that is conjugated with a detectable marker.

In another example, the cellular proteins are isolated and subjected to SDS-PAGE followed by Western blotting. After resolving the proteins, the proteins are transferred to a membrane, which is probed with specific binding agents that recognize FGF-5. The proteins are detected, for example with HRP-conjugated secondary antibodies, and quantitated.

Quantitation of FGF-5 proteins can be made by immunoassay and compared to levels of the protein found in non-FGF-5 expressing cells, such as normal adult tissues, or to the level of FGF-5 in healthy, normal cells (for example, cells of the same origin that are not neoplastic or are free of the disease of interest). A significant (for example 50% or greater) increase in the amount of FGF-5 protein in the tumor cells compared to the amount of FGF-5 protein found in non-FGF-5 expressing cells or that found in normal cells, indicates that the tumor is an FGF-5 expressing or overexpressing tumor.

### EXAMPLE 16

### Administration of Both an FGF-5 HLA-A3 and a -A2 Epitope

This example describes methods that can be used to stimulate an immune response by administering both an FGF-5 HLA-A2 and -A3 epitope.

For example, using the methods described in Example 10, a combination of FGF-5 HLA-A3 and -A2 epitopes can administered to a subject at a therapeutically effective dose. In a particular example, subjects having both an HLA-A2⁺ and HLA-A3⁺ allele are vaccinated subcutaneously with 1 mg of an FGF-5 HLA-A2 peptide (such as a sequence that includes SEQ ID NO: 32 or a variant, fragment, or fusion thereof) and 1 mg of an FGF-5 HLA-A3 peptide (such as a sequence that includes any of SEQ ID NOS: 26-31, 34 and 39, or a variant, fragment, or fusion thereof) in the presence of an adjuvant (such as Montanide ISA-51). The vaccinations can be repeated as needed, fro example, daily for four days every 3 weeks for up to a year (or until tumor progression is documented), or for example daily for four days every 2 months for up to a year (or until tumor progression is documented).

In addition to the FGF-5 HLA-A3 and -A2 peptides, other agents can be administered to the subject, such as IL-2 (for example as described in Example 10). Following administration of the FGF-5 HLA-A2 and -A3 peptides, the FGF-5 expressing or overexpressing tumor can be monitored for regression, for example using the methods described herein. In addition, whether an immune response was stimulated (for example by measuring IFN-γ production from PBMCs) can be determined using the methods described in Example 11.

### EXAMPLE 17

### Immunogenic Compositions and Modes of Administration

Administering the therapeutic agents disclosed herein, such as the FGF-5 HLA-A2 and HLA-A3 epitopes of the present disclosure, antineoplastic agents such as IL-2 and immunoreactive T cells sensitized with an FGF-5 epitope, can be accomplished by any means known to the skilled artisan. Methods of introduction include, but are not limited to, intradermal, intramuscular, intraperitoneal, intravenous, subcutaneous, intranasal, and oral routes. Compounds can be administered by any convenient route, for example by infusion or bolus injection, by absorption through epithelial or mucocutaneous linings (such as oral mucosa, rectal and intestinal mucosa, etc.) and can be administered together with other biologically active agents. Administration can be systemic or local. Continuous infusion may also be appropriate. In one example, administration is by direct injection at the site (or former site) of an FGF-5 expressing-tumor or neoplastic or pre-neoplastic tissue. For a brief review of methods for drug delivery, see Langer, Science 249:1527-33, 1990.

Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives or auxiliaries such as disintegrants, binders, coating agents, swelling agents, lubricants, flavorings, sweeteners or solubilizers are customarily used as described above.

The disclosed FGF-5 HLA-A2 and HLA-A3 epitopes (either as a peptide or as a nucleic acid molecule that encodes the peptide) can be administered to a subject directly, or can be present in a pharmaceutically acceptable carrier. However, a pharmaceutically acceptable carrier may not be required to induce an immune response to the FGF-5 HLA-A2 or HLA-A3 epitope. In general, the nature of the pharmaceutically acceptable carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Thus, a wide variety of pharmaceutically acceptable carriers are acceptable, and include materials which are inert, those having biological activity (such as an anti-neoplastic agent), or those that promote an immune response (such as an adjuvant). In one example, a pharmaceutical compostion includes only one type of therapeutic molecule (such as an FGF-5 HLA-A2 or -A3 epitope), or can include a combination of several types of therapeutic molecules, such as other anti-neoplasic agents, such as IL-2.

A particular example of a pharmaceutically acceptable carrier is an agent that aids in stimulation of the immune response, such as an adjuvant. Adjuvants are nonspecific immune stimulators that increase the immune readiness and aid in stimulating a higher level (titer) of serum antibodies that recognize the epitopic peptide sequences. Adjuvants include, for example, Freund's complete adjuvant, Freund's incomplete adjuvant, B30-MDP, LA-15-PH, Montanide, saponin, aluminum hydroxide, alum, lipids, keyhole lympet protein, hemocyanin, a mycobacterial antigen, and combinations thereof. If the adjuvant is a lipid it can be linked to the epitopic peptide(s).

Other examples of pharmaceutically acceptable carriers include physiologically acceptable masses to which the eptiope it attached, and in some examples, enhances the immune response. In one example, a mass is one or more amino acids or other moieties, such as a dimer, oligomer, or higher molecular weight polymer of a sequence of amino acids of an FGF-5 HLA-A2 and HLA-A3 epitope. In other words, an FGF-5 HLA-A2 or HLA-A3 epitope can be formed from naturally available materials or synthetically produced and can then be polymerized to build a chain of two or more repeating units so that the repeating sequences form both the carrier and the immunogenic polypeptide. Alternatively, additional amino acids can be added to one or both ends of an FGF-5 HLA-A2 and HLA-A3 epitope. Polysaccharides can also attached to the disclosed epitopes, and include those of molecular weight 10,000 to 1,000,000, such as starches, dextran, agarose, ficoll, or its carboxylmethyl derivative and carboxy methyl cellulose. Polyamino acids can also attached to the disclosed epitopes, and include, polylysine, polyalanyl polylysine, polyglutamic acid, polyaspartic acid and poly (C₂-C₁₀) amino acids.

Organic polymers can also attached to the disclosed epitopes, and these polymers include, for example, polymers and copolymers of amines, amides, olefins, vinyls, esters, acetals, polyamides, carbonates and ethers and the like. Generally speaking, the molecular weight of these polymers will vary dramatically. The polymers can have from two repeating units up to several thousand, such as two thousand repeating units. The number of repeating units will be consistent with the use of the immunizing composition in a host animal. Usually, such polymers have a lower molecular weight, for example, between 10,000 and 100,000 kD (the molecular weight being determined by ultracentrifugation). Inorganic polymers can also be employed. These inorganic polymers can be inorganic polymers containing organic moieties. In particular, silicates and aluminum hydroxide can be used as carriers. Ideally, the carrier is one which is an immunological adjuvant. In such cases, it is contemplated that the adjuvant be muramyl dipeptide or its analogs.

In one example, FGF-5 HLA-A2 and HLA-A3 eptitopic peptides are administered to a subject in a colloidal dispersion system. Colloidal dispersion systems include macromolecule complexes, nanocapsules, microspheres, beads, and lipid-based systems including oil-in-water emulsions, micelles, mixed micelles, and liposomes.

Liposomes are artificial membrane vesicles that are useful as delivery vehicles in vitro and in vivo. Large uni-lamellar vesicles (LUV), which range in size from 0.2-4.0 μm, can encapsulate a substantial percentage of an aqueous buffer containing large macromolecules. Nucleic acids encoding for an FGF-5 HLA-A2 or HLA-A3 epitope can be encapsulated within the aqueous interior and be delivered to cells in a biologically active form (Fraley et al., 1981, Trends Biochem. Sci. 6:77, 1981). The composition of the liposome is usually a combination of phospholipids, such as high-phase-transition-temperature phospholipids, usually in combination with steroids, especially cholesterol. However, other phospholipids or other lipids can also be used. In one example, a liposome includes the desired FGF-5 HLA-A2 or HLA-A3 eptiope and is directed to the site of lymphoid cells, where the liposomes then deliver the selected therapeutic epitope. The lipids can be directly attached to the epitope peptide or indirectly through a linkagee. For example, a lipid can be attached directly to the amino terminus of the peptide or via a linkage such as Ser-Ser, Gly, Gly-Gly, or Ser.

Nucleic acid molecules that encode an FGF-5 HLA-A2 or HLA-A3 epitope can be part of a vector, which is administered to a subject. For example, viral vectors include, but are not limited to, adenovirus, herpes virus, vaccinia, or an RNA virus such as a retrovirus. For example, by inserting a nucleic acid sequence encoding an FGF-5 HLA-A2 or HLA-A3 peptide into a viral vector, along with another gene that encodes the ligand for a receptor on a specific target cell (such as an FGF-5 expressing tumor cell), the vector is now target specific.

Retroviral vectors can be made target specific by attaching, for example, a sugar, a glycolipid, or a protein (such as an antibody). Since recombinant retroviruses are defective, they need assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include, but are not limited to Q2, PA317, and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

In one example, administration of an FGF-5 HLA-A2 or -A3 epitope peptide is through ingestion. In particular examples, for oral administration, a pharmaceutically acceptable nontoxic composition is formed by incorporating any of the normally employed excipients, such as those carriers previously listed, and generally about 10-95% of FGF-5 HLA-A2 or -A3 epitope peptide, for example, at a concentration of about 25%-75%. In addition, pill-based forms of pharmaceutical proteins can be administered subcutaneously, for example if formulated in a slow-release composition. Slow-release formulations may be produced by combining the protein with a biocompatible matrix, such as cholesterol. Another possible method of administering protein pharmaceuticals is through the use of mini osmotic pumps.

In another example, the disclosed epitope peptide compositions are administered in finely divided form along with a surfactant and propellant. Typical percentages of peptides are about 0.01%—about 20% by weight, for example, about 1%—about 10%. The surfactant ideally is nontoxic and soluble in the propellant. Representative surfactants include the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may also be employed. The surfactant can constitute about 0.1%—about 20% by weight of the composition, for example, about 0.25—about 5% by weight of the composition. The balance of the composition is typically propellant. A carrier can also be included, as desired. For example, lecithin can be used for intranasal delivery.

The pharmaceutical compositions disclosed herein can be prepared and administered in dose units. For treatment of a subject, depending on activity of the compound, manner of administration, nature and severity of the disorder, age and body weight of the patient, different daily doses are necessary. Under certain circumstances, however, higher or lower daily doses may be appropriate. The administration of a therapeutic amount can be carried out both by single administration in the form of an individual dose unit or else several smaller dose units and also by multiple administration of subdivided doses at specific intervals.

Amounts effective for therapeutic use can depend on the severity of the disease and the age, weight, general state of the patient, and other clinical factors. Thus, the final determination of the appropriate treatment regimen will be made by the attending clinician. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman and Gilman: The Pharmacological Bases of Therapeutics, 8th ed., Pergamon Press, 1990; and Remington's Pharmaceutical Sciences, 17^{th} ed., Mack Publishing Co., Easton, Pa., 1990.

In one example, the dose range for an FGF-5 HLA-A2 or HLA-A3 epitope protein is from about 0.1 mg to about 10 mg. In one example, the dose is about 0.1 mg to about 5 mg, for example, 1 mg to 5 mg, such as 1 mg peptide per subject. The dosing schedule can vary from daily to as seldom as once a year, depending on clinical factors, such as the subject's sensitivity to the peptide and tempo of their disease. Therefore, a subject can receive a first dose of immunogenic FGF-5 HLA-A2 or HLA-3 epitope, and then receive a second dose (or even more doses) at some later time(s), such as at least one day later, such as at least one week later. In one example, initial immunization can be followed by boosting dosages of from 0.1 mg to about 10 mg, for example, 0.1 mg to about 5 mg, for example, 1 mg to 5 mg, such as 1 mg. A boosting regimen can be followed over weeks to months, depending upon the subject's response and condition by measuring specific immune activity in the patient.

The disclosure also provides a pharmaceutical pack or kit that includes one or more containers filled with one or more of the disclosed pharmaceutical compositions.

### EXAMPLE 18

### Sequence Variants

Having disclosed FGF-5 HLA-A2 and -A3 epitope nucleic acid and protein sequences, this disclosure facilitates the creation of nucleic acid molecules and proteins derived from those disclosed but which vary in their precise nucleotide or amino acid sequence from those disclosed. Such variants can be obtained through a combination of standard molecular biology laboratory techniques and the sequence information disclosed herein. Thus, the methods disclosed herein may be practiced with molecules that differ from the exact FGF-5 HLA-A2 and -A3 sequences disclosed, but which retain the ability to stimulate an immune response, for example against an FGF-5 expressing tumor. Methods of administration and particular doses are disclosed herein (see Example 17).

Protein Sequences

Variants of FGF-5 HLA-A2 (SEQ ID NO: 32) and -A3 (SEQ ID NOS: 26-31, 34, and 39) epitopes that retain their immunogenicity, can be generated using standard methods. The ability to stimulate an immune response, for example, an HLA-A3- or -A2 restricted CTL immune response (for example against an FGF-5 expressing or over-expressing tumor), can be determined using methods known in the art, such as the ability to stimulate IFN-γ production using the assay described in Examples 2 and 11.

Substitutional variants are those in which at least one residue in the amino acid sequence has been removed and a different residue inserted in its place. The simplest modifications involve the substitution of one or more amino acid residues (for example 1, 2, or 3 residues) for amino acid residues having similar biochemical properties. These conservative substitutions are likely to have minimal impact on the activity of the resultant protein. Another example of a variant is on that includes insertion or deletion of amino acids, such as insertions of 1, 2, or 3 amino acid residues within a sequences, or even longer on the N- or C-terminus of the protein; and deletions of 1, 2, or 3 residues. One skilled in the art will understand that combinations of such variations can be made.

Particular examples of substitutions that can be made to an FGF-5 HLA-A2 epitope sequence include, but are not limited to: position 9 (A→S or V) and 10 (V→A) (positions refer to nucleotide numbers of SEQ ID NO: 32). Ideally, a L2F substation is not included in SEQ ID NO: 32. Particular examples of substitutions that can be made to an FGF-5 HLA-A3 epitope sequence include, but are not limited to: positions 1 (N→A), 2 (T→A), 5 (S→A), 6 (P→A), and an insertion of Leu between amino acids 5 and 6 (positions refer to nucleotide numbers of SEQ ID NO: 26).

Proteins that include an FGF-5 HLA-A2 and -A3 epitope variant or fragment in particular examples retain at least 77%, at least 80%, at least 87%, or at least 90%, sequence identity to the FGF-5 HLA-A2 and -A3 epitope sequences disclosed herein, and in particular examples at least this much identity to SEQ ID NOS: 26-32, 34, and 39.

Nucleic Acid Molecules

Variant nucleic acid molecules, such as those that encode variant FGF-5 HLA-A2 and -A3 epitope sequences, can be generated using standard mutagenesis techniques, for example, M13 primer mutagenesis (for example see Sambrook et al., In: Molecular Cloning: A Laboratory Manual, Cold Spring Harbor, N.Y., 1989, Chapter 15). In particular examples, a variant FGF-5 HLA-A2 and -A3 epitope nucleic acid molecules include those that can specifically hybridize under stringent conditions (such as high stringency of very high stringency conditions) to a nucleic acid sequence that encodes an FGF-5 HLA-A2 and -A3 epitope sequence. Specific hybridization refers to the binding, duplexing, or hybridizing of a molecule only or substantially only to a particular nucleotide sequence when that sequence is present in a complex mixture (such as total cellular DNA or RNA).

The degeneracy of the genetic code enables major variations in the nucleotide sequence of a DNA molecule while maintaining the amino acid sequence of the encoded protein. For example, the 9th amino acid residue of an FGF-5 HLA-A2 epitope (SEQ ID NO: 32) is alanine. This is encoded in the FGF-5 HLA-A2 cDNA by the nucleotide codon triplet GCT (see SEQ ID NO: 3). Due to the degeneracy of the genetic code, three other nucleotide codon triplets, GCC, GCA and GCG, also code for alanine. Thus, the nucleotide sequence of an FGF-5 HLA-A2 epitope can be changed at this position to any of these three codons without affecting the amino acid composition of the encoded protein or the characteristics of the protein. Similar substitutions can be made throughout an FGF-5 HLA-A2 or -A3 epitope nucleic acid sequence. Based upon the degeneracy of the genetic code, variant DNA molecules may be derived from the nucleic acid molecules disclosed herein using standard DNA mutagenesis techniques or by synthesis of DNA sequences. DNA sequences which do not hybridize under stringent conditions to the cDNA sequences disclosed by virtue of sequence variation based on the degeneracy of the genetic code are herein also comprehended by this disclosure.

An example of a nucleic acid sequence that encodes an FGF-5 HLA-A2 epitope includes nucleotides 488-517 of SEQ ID NO: 3. Particular examples of substitutions that can be made to an FGF-5 HLA-A2 epitope sequence include, but are not limited to: positions 493 (A→G), 496 (T→C), 499 (T→C, A, or G), 508 (A→C or T) and 517 (G→C, A, or T) (positions refer to nucleotide numbers of SEQ ID NO: 3). An example of a nucleic acid sequence that encodes an FGF-5 HLA-A3 epitope includes nucleotides 663-667 and 788-799 of SEQ ID NO: 3. Particular examples of substitutions that can be made to an FGF-5 HLA-A3 epitope sequence include, but are not limited to: positions 655 (T→C), 667 (A→C, T, or G), 790 (A→C, T, or G), 796 (C→T), and 799 (G→A) (positions refer to nucleotide numbers of SEQ ID NO: 3).

FGF-5 HLA-A2 and -A3 epitope variant nucleic acid molecules in particular examples retain at least 70%, at least 80%, at least 90%, or at least 95%, sequence identity to the FGF-5 HLA-A2 and -A3 epitope nucleic acid sequences disclosed herein, and in particular examples at least this much identity to nucleic acid sequences that encode SEQ ID NOS: 26-32, 34, and 39.

### EXAMPLE 19

### FGF-5 HLA-A2 and -A3 Fusion Proteins

This example describes methods that can be used to generate a fusion protein that includes an FGF-5 HLA-A2 or -A3 epitope (or both epitopes). Methods for making fusion proteins are well known (for example, see U.S. Pat. Nos. 6,057,133 and 6,072,041).

Fusion proteins include an FGF-5 HLA-A2 or -A3 epitope linked to other amino acid sequences, such as 1-50 amino acids added to the N- or C-terminus (or both) of the epitope sequence. Linker regions can be used to space the two portions of the protein from each other and to provide flexibility between them. The linker region can be a peptide of between 1 and 500 amino acids, for example less than 30 amino acids. The linker joining the two molecules can be designed to (1) allow the two molecules to fold and act independently of each other, (2) not have a propensity for developing an ordered secondary structure which could interfere with the functional domains of the two proteins, (3) have minimal hydrophobic or charged characteristic which could interact with the functional protein domains and (4) provide steric separation of the two regions. Examples of amino acids that can be used in the linker include Gly, Asn, Ser, Thr and Ala. Additional amino acids may also be included in the linker due to the addition of unique restriction sites in the linker sequence to facilitate construction of the fusions. Other moieties can also be included, such as a binding region (for example avidin or a polyhistadine tag) and detectable labels (such as radionuclides, enzymes, fluors, and the like).

Fusion proteins can be generated using chemical synthesis, or by recombinant DNA technologies. For example, nucleic acid molecules encoding one peptide, peptide linker, and the other peptide can be inserted into a suitable expression vector which is used to transform prokaryotic or eukaryotic cells, for example bacteria, yeast, insect cells or mammalian cells (see EXAMPLE 20). The transformed organism is grown and the protein isolated, for example by using a detectable marker such as nickel-chelate affinity chromatography, if a polyhistadine tag is used.

### EXAMPLE 20

### Recombinant Expression of FGF-5

With the provision of FGF-5 HLA-A2 and -A3 epitope sequences, the expression and purification of FGF-5 HLA-A2 and -A3 epitope proteins by recombinant techniques is now enabled. The purified protein can be administered to a subject to stimulate an immune response in the subject, such as an immune response against an FGF-5 expressing or overexpressing tumor.

FGF-5 HLA-A2 and -A3 epitope cDNA sequences can be ligated into expression vectors. Methods and plasmid vectors for producing fusion proteins and intact native proteins in bacteria are described in Sambrook et al. (Molecular Cloning: A Laboratory Manual, Cold Spring Harbor, N.Y., 1989, Chapter 17). Uptake of nucleic acids by a prokaryotic cell, such as E. coli, can be achieved using competent cells treated by the CaCl₂ method. Alternatively, MgCl₂ or RbCl can be used. Transformation can also be performed by electroporation.

Vectors suitable for the production of intact native proteins include pKC30 (Shimatake and Rosenberg, 1981, Nature 292:128), pKK177-3 (Amann and Brosius, 1985, Gene 40:183), T7-based expression vectors (Rosenberg et al., 1987, Gene 56:125), pMSXND (Lee and Nathans, J. Biol. Chem. 263:3521, 1988), baculovirus-derived vectors, and pET-3 (Studiar and Moffatt, 1986, J. Mol. Biol. 189:113). The FGF-5 HLA-A2 or -A3 epitope cDNA sequence can also be transferred to other cloning vehicles, such as other plasmids, bacteriophages, cosmids, animal viruses and yeast artificial chromosomes (YACs) (Burke et al., 1987, Science 236:806-12). These vectors may then be introduced into a variety of hosts including somatic cells, and simple or complex organisms, such as bacteria, fingi (Timberlake and Marshall, 1989, Science 244:1313-7), invertebrates, plants (Gasser and Fraley, 1989, Science 244:1293), and mammals (Pursel et al., 1989, Science 244:1281-8). For example, large amounts of an FGF-5 HLA-A2 or -A3 epitiope can be produced in E. coli, and subsequently purified. Similar methods can be used to produce an FGF-5 HLA-A2 or -A3 fusion proteins, such as an FGF-5 HLA-A2 or -A3 epitiope that includes N- or C-terminal peptides.

For expression in mammalian cells, the cDNA sequence can be ligated to heterologous promoters, such as the simian virus SV40, promoter in the pSV2 vector (Mulligan and Berg, 1981, Proc. Natl. Acad. Sci. USA 78:2072-6), and introduced into cells, such as monkey COS-1 cells (Gluzman, 1981, Cell 23:175-82), Chinese hamster ovary (CHO), mouse NIH 3T3 fibroblasts, human fibroblasts, or lymphoblasts, to achieve transient or long-term expression. The stable integration of the chimeric gene construct can be maintained in mammalian cells by biochemical selection, such as neomycin (Southern and Berg, 1982, J. Mol. Appl. Genet. 1:327-41) and mycophoenolic acid (Mulligan and Berg, 1981, Proc. Natl. Acad. Sci. USA 78:2072-6). In one example, an FGF-5 HLA-A2 and -A3 epitope sequence is expressed in mammalian cells using the vector pXT1 (Stratagene). Eukaryotic cells can also be cotransformed with nucleic acid molecules encoding an FGF-5 HLA-A2 or HLA-A3 epitope, and a second foreign DNA molecule encoding a selectable phenotype, such as the herpes simplex thymidine kinase gene.

The level of expression of the cDNA can be manipulated by using promoters that have different activities (for example, the baculovirus pAC373 can express cDNAs at high levels in S. frugiperda cells (Summers and Smith, 1985, Genetically Altered Viruses and the Environment, Fields et al. (Eds.) 22:319-328, Cold Spring Harbor Laboratory Press, Cold Spring Harbor, N.Y.) or by using vectors that contain promoters amenable to modulation, for example, the glucocorticoid-responsive promoter from the mouse mammary tumor virus (Lee et al., 1982, Nature 294:228).

The transfer of DNA into eukaryotic, such as human or other mammalian cells is conventional. Vectors can be introduced into recipient cells as pure DNA (transfection) by, for example, precipitation with calcium phosphate (Graham and vander Eb, 1973, Virology 52:466) or strontium phosphate (Brash et al., 1987, Mol. Cell Biol. 7:2013), electroporation (Neumann et al., 1982, EMBO J. 1:841), lipofection (Felgner et al., 1987, Proc. Natl. Acad. Sci USA 84:7413), DEAE dextran (McCuthan et al., 1968, J. Natl. Cancer Inst. 41:351), microinjection (Mueller et al., 1978, Cell 15:579), protoplast fusion (Schafner, 1980, Proc. Natl. Acad. Sci. USA 77:2163-7), or pellet guns (Klein et al., 1987, Nature 327:70). Alternatively, the cDNA can be introduced by infection with virus vectors, such as retroviruses (Bernstein et al., 1985, Gen. Engrg. 7:235), adenoviruses (Ahmad et al., 1986, J. Virol. 57:267), or Herpes virus (Spaete et al., 1982, Cell 30:295).

### EXAMPLE 21

### Peptide Synthesis and Purification

As an alternative to producing the disclosed FGF-5 HLA-A2 and -A3 epitiopes recombinantly, chemical synthesis of the peptides can be used. Peptides that include disclosed FGF-5 HLA-A2 and -A3 epitiopes (and variants, fusions and fragments thereof) can be chemically synthesized by any of a number of manual or automated methods of synthesis known in the art. For example, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. (Solid Phase Peptide Synthesis, IRL Press: Oxford, 1989).

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at RT.

Crude peptides are purified by preparative high pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

### EXAMPLE 22

### Peptide Modifications

The disclosed FGF-5 HLA-A2 and -A3 sequences can be modified, while retaining an ability to generate an immune response, such as in a subject in whom the peptide is administered. Exemplary modifications include, but are not limited to, FGF-5 antigenic epitope analogues (non-peptide organic molecules), derivatives (chemically functionalized peptide molecules obtained starting with the disclosed peptide sequences) and variants (homologs) that generate an immune response. The disclosed peptides include a sequence of amino acids, which can be either L- and/or D-amino acids, naturally occurring and otherwise.

FGF-5 antigenic epitope peptide sequences can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the disclosed peptides to select and provide conformational constraints to the structure that result in enhanced stability.

Peptidomimetic and organomimetic embodiments are also within the scope of the present disclosure, whereby the three-dimensional arrangement of the chemical constituents of such peptido- and organomimetics mimic the three-dimensional arrangement of the peptide backbone and component amino acid side chains, resulting in such peptido- and organomimetics of the proteins disclosed herein having measurable or enhanced ability to bind an antibody. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included within the scope of the disclosure are mimetics prepared using such techniques. In one example, a mimetic mimics the immune response generated by an FGF-5 HLA-A3 or HLA-A2 epitope.

Suitable modified proteins have the ability to elicit or stimualte an immune response. Such modified FGF-5 HLA-A3 or HLA-A2 epitopes can be used to stimulate an immune response in a subject, for example using the methods described in Examples 9 and 10.

### EXAMPLE 23

### In Vivo Expression of FGF-5 Epitope Nucleic Acid Molecules

This example describes methods that can be used to express an FGF-5 HLA-A3 or HLA-A2 epitope in vivo, for example to stimulate an immune response in a subject, such as to treat an FGF-5 expressing or overexpressing tumor.

Administration of a nucleic acid sequence encoding an FGF-5 HLA-A3 or HLA-A2 epitope to a subject suffering from a tumor that expresses or over-expresses FGF-5 may further enhance the immune response, such as stimulating CTLs to lyse FGF-5 expressing tumor cells, and achieve the desired therapeutic goal of treating the tumor. In some subjects, expression or overexpression of FGF-5 by the tumor may be insufficient to stimulate the immune system.

In one example, antigen presenting cells (APCs, such as dendritic cells) are removed from a subject having from a tumor that expresses or over-expresses FGF-5. The APCs are transfected with an expression vector containing FGF-5 HLA-A3 or HLA-A2 epitope cDNA. These transfected cells will thereby produce the functional FGF-5 epitope, and can be reintroduced into the subject to stimulate CTLs. A general strategy for transferring genes into donor cells is disclosed in U.S. Pat. No. 5,529,774.

In another example, an FGF-5 HLA-A3 or HLA-A2 epitope cDNA is administered to a subject, for example in a non-infectious form (such as naked DNA or liposome encapsulated DNA), or as part of a vector. For example, a sequence encoding an FGF-5 HLA-A3 or HLA-A2 epitope can be cloned into a viral expression vector (such as avipox viruses, recombinant vaccinia virus, replication-deficient adenovirus strains or poliovirus) and that vector is introduced into the subject. The virus infects the cells, and produces the protein sequence in vivo, where it has its desired therapeutic effect (see, for example, Zabner et al. Cell 75:207-16, 1993). The FGF-5 HLA-A3 or HLA-A2 epitope cDNA can be administered to the subject by any method which allows the nucleic acid molecule to reach the appropriate cells, such as injection, infusion, deposition, implantation, or topical administration.

The nucleic acid molecules can be delivered to particular cells or tissues (such as an FGF-5 expressing tumor). However, in some examples it may be more therapeutically effective to treat all of the subject's cells, or more broadly disseminate the nucleic acid molecule, for example by intravascular administration.

The nucleic acid sequence encoding an FGF-5 HLA-A3 or HLA-A2 epitope can be under the control of a suitable promoter, such as an FGF-5 promoter, retroviral LTR promoter, or adenoviral promoters, such as the adenoviral major late promoter; the cytomegalovirus (CMV) promoter; the Rous Sarcoma Virus (RSV) promoter; inducible promoters, such as the MMTV promoter; the metallothionein promoter; heat shock promoters; the albumin promoter; the histone promoter; the α-actin promoter; TK promoters; B19 parvovirus promoters; and the ApoAI promoter. However the scope of the disclosure is not limited to specific promoters.

Exemplary Viral Vectors

Nucleic acid sequences encoding an FGF-5 HLA-A3 or HLA-A2 epitope can be part of a viral vector. Adenoviral vectors can include essentially the complete adenoviral genome (Shenk et al., Curr. Top. Microbiol. Immunol. 111: 1-39, 1984), or can be a modified adenoviral vector in which at least a portion of the adenoviral genome has been deleted. In one example, the vector includes an adenoviral 5′ ITR; an adenoviral 3′ ITR; an adenoviral encapsidation signal; a DNA sequence encoding a therapeutic agent; and a promoter for expressing the DNA sequence encoding a therapeutic agent. The vector is free of at least the majority of adenoviral E1 and E3 DNA sequences, but is not necessarily free of all of the E2 and E4 DNA sequences, and DNA sequences encoding adenoviral proteins transcribed by the adenoviral major late promoter. In another example, the vector is an adeno-associated virus (AAV) such as described in U.S. Pat. No. 4,797,368.

Such a vector can be constructed according to standard techniques, using a shuttle plasmid which contains, beginning at the 5′ end, an adenoviral 5′ ITR, an adenoviral encapsidation signal, and an E1a enhancer sequence; a promoter (which may be an adenoviral promoter or a foreign promoter); a tripartite leader sequence, a multiple cloning site (which may be as herein described); a poly A signal; and a DNA segment which corresponds to a segment of the adenoviral genome. The DNA segment serves as a substrate for homologous recombination with a modified or mutated adenovirus, and may encompass, for example, a segment of the adenovirus 5′ genome no longer than from base 3329 to base 6246. The plasmid can also include a selectable marker and an origin of replication. The origin of replication may be a bacterial origin of replication. A desired DNA sequence encoding a therapeutic agent may be inserted into the multiple cloning site of the plasmid.

The plasmid may be used to produce an adenoviral vector by homologous recombination with a modified or mutated adenovirus in which at least the majority of the E1 and E3 adenoviral DNA sequences have been deleted. Homologous recombination can be effected through co-transfection of the plasmid vector and the modified adenovirus into a helper cell line, such as 293 cells, by CaPO₄ precipitation. The homologous recombination produces a recombinant adenoviral vector which includes DNA sequences derived from the shuttle plasmid between the Not I site and the homologous recombination fragment, and DNA derived from the E1 and E3 deleted adenovirus between the homologous recombination fragment and the 3′ ITR.

In one example, an adenovirus is constructed by using a yeast artificial chromosome (or YAC) containing an adenoviral genome according to the method described in Ketner et al. (Proc. Natl. Acad. Sci. USA, 91:6186-90, 1994), in conjunction with the teachings contained herein. In this example, the adenovirus yeast artificial chromosome is produced by homologous recombination in vivo between adenoviral DNA and yeast artificial chromosome plasmid vectors carrying segments of the adenoviral left and right genomic termini. A DNA sequence encoding an FGF-5 HLA-A3 or HLA-A2 epitope can then be cloned into the adenoviral DNA. The modified adenoviral genome then is excised from the adenovirus yeast artificial chromosome in order to be used to generate adenoviral vector particles as hereinabove described.

Viral particles that include an FGF-5 HLA-A3 or HLA-A2 epitope are administered in an amount effective to produce a therapeutic effect in a subject. The viral particles can be administered as part of a preparation having a titer of viral particles of at least 1×10¹⁰ pfu/ml, for example not exceeding 2×10¹¹ pfu/ml. The viral particles can be administered in combination with a pharmaceutically acceptable carrier in a volume up to 10 ml.

In another example, the viral vector is a retroviral vector. FGF-5 HLA-A3 or HLA-A2 epitopes can be cloned into a retroviral vector and driven from either its endogenous promoter or from the retroviral LTR (long terminal repeat). Examples of retroviral vectors include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, gibbon ape leukemia virus (GaLV), and mammary tumor virus. The vector is generally a replication defective retrovirus particle.

Retroviral vectors are useful as agents to effect retroviral-mediated gene transfer into eukaryotic cells. Retroviral vectors are generally constructed such that the majority of sequences coding for the structural genes of the virus are deleted and replaced by the gene(s) of interest. Most often, the structural genes (such as gag, pol, and env) are removed from the retroviral backbone.

Other viral transfection systems can also be utilized, including Vaccinia virus (Moss et al., 1987, Annu. Rev. Immunol. 5:305-24), Bovine Papilloma virus (Rasmussen et al., 1987, Methods Enzymol. 139:642-54) or members of the herpes virus group such as Epstein-Barr virus (Margolskee et al., 1988, Mol. Cell. Biol. 8:2837-47). RNA-DNA hybrid oligonucleotides, as described by Cole-Strauss et al. (Science 273:1386-9, 1996), can also be used.

FGF-5 HLA-A3 or HLA-A2 epitope nucleic acid sequences can be incorporated into proviral backbones in several general ways. In the most straightforward constructions, the structural genes of the retrovirus are replaced by a single gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

### EXAMPLE 24

### Methods for Measuring an Immune Response

Several methods known to those skilled in the art can be used to monitor an immune response in a subject. Although this example provides specific examples of assays which can be used for this purpose, other methods can be used to measure an immune response.

For example, elicitation or stimulation of an immune response can be measured by a observing a change in the activity or number of T-cells in the peripheral blood of a subject, or by measuring IFN-γ concentration, using the assays described in EXAMPLES 2, 3, and 11. For example, when an immune response is increased, the concentration of IFN-γ may increase by a desired amount, for example by at least 50%, at least 75% or even at least 1000%.

### EXAMPLE 25

### Testing of Non-Native Epitope Sequences

This example describes methods that can be used to determine if an agent can stimulate the desired immune response. Such methods can be used to determine if a variant, fragment, or fusion of a native FGF-5 HLA-A2 or HLA-A3 peptide sequence (or nucleic acid encoding such a peptide), retains the desired biological activity. In addition, such methods can be used to determine if an immunogenic composition has the ability to stimulate the desired immune response.

Proliferative assays can be used to measure the ability of an FGF-5 HLA-A2 or HLA-A3 peptide variant, fragment, or fusion to stimulate a T-cell response (for example see PCT publication WO 02/22860). Briefly, T-cells (2×10⁴) or irradiated peripheral blood mononuclear cells (5×10⁴) are seeded, in duplicate, into wells with or without about 200 μg/ml peptide. Proliferation is measured by ³H-thymidine incorporation (Hemmer et al., 1997, J. Exp. Med. 185:1651-9). In one example, a therapeutic composition that includes an FGF-5 HLA-A2 or HLA-A3 peptide is one that can increase a T-cell response (proliferation) by at least about 10%, for example at least about 20%, or even about 50%, for example as compared to an amount of proliferation in the absence of the HLA-A2 or HLA-A3 peptide.

FGF-5 HLA-A2 and HLA-A3 variant, fragment, or fusion peptides can also be tested in a cytotoxic T lymphocyte (CTL) assay (see, for example, Sette et al., 1994. J. Immunol. 153:5586-92, and PCT publication WO 01/55177). Briefly, the spleen of peptide immunized transgenic mice are collected aseptically 10 days after immunization and placed in 5 ml of cell medium (RMPI 1640, penicillin+streptomycin, 2% Hepes buffer, 10% Fetal calf serum) on ice. The splenocytes are cultured for 6 days in the presence of LPS blasts coated with 100 μg/ml of the peptide (stimulator cells) and then assayed for peptide-specific CTL activity by using EL4-A2 and EL4 cell lines in the presence or absence of the query peptides. In one example, a therapeutic composition that includes an FGF-5 HLA-A2 or HLA-A3 peptide is one that can increase peptide-specific CTL activity by at least about 10%, for example at least about 20%, or even about 50%, for example as compared to an amount of CTL activity in the absence of the HLA-A2 or HLA-A3 peptide.

### EXAMPLE 26

### Determination of a Therapeutically Effective Amount

This example describes methods that can be used to identify therapeutically effective doses of an FGF-5 HLA-A3 epitope, FGF-5 HLA-A2 epitope, immunoreactive sensitized T cells sensitized with such an epitope, or combinations thereof. In one example, a desired response is stimulation of a CTL response to an FGF-5 expressing or over-expressing tumor, such as RCC, resulting in halting or slowing the progression of, or inducing a regression of a pathological condition or which is capable of relieving signs or symptoms caused by the condition. One example of a therapeutic effect is regression of the tumor, lysis of the cells of the tumor, or both. Treatment can involve only slowing the progression of the disease temporarily, but can also include halting or reversing the progression of the disease permanently.

In another or additional example, it is an amount sufficient to increase the efficacy of another agent, such as IL-2. In one example, the efficacy of IL-2 is increased by at least 10%, for example at least 20%, in the presence of another agent, as measured by a clinical response.

The therapeutically effective amount also includes a quantity of FGF-5 HLA-A3 or HLA-A2 epitope protein or nucleic acid molecules (such as a sequence that includes SEQ ID NO: 26-32, 34 or 39, or variants or fragments thereof), autologous CTLs specific to FGF-5, or combinations thereof sufficient to achieve a desired effect in a subject being treated. For instance, this can be the amount necessary to improve signs or symptoms a disease such as cancer, for example by modulating, for example increasing a CTL response against a tumor expressing or overexpressing FGF-5.

An effective amount of FGF-5 HLA-A3 or HLA-A2 epitope protein (or nucleic acid), autologous CTLs specific to FGF-5, or combinations thereof, can be administered in a single dose, or in several doses, for example daily, during a course of treatment. However, the effective amount of can be dependent on the source applied (for example FGF-5 peptide isolated from a cellular extract versus a chemically synthesized and purified FGF-5 peptide, or a variant or fragment that may not retain full FGF-5 activity), the subject being treated, the severity and type of the condition being treated, and the manner of administration. In one example, a therapeutically effective amount of FGF-5 HLA-A3 or HLA-A2 epitope protein varies from about 0.01 mg to about 10 mg, such as about 1 mg per subject.

In view of the many possible embodiments to which the principles of our disclosure may be applied, it is to be recognized that the illustrated embodiments are only examples of the disclosure and are not be taken as a limitation on the scope of the disclosure. Rather, the scope of the disclosure is defined by the following claims. We therefore claim as our invention all that comes within the scope and spirit of these claims.

We claim:

1. A purified immunogenic peptide comprising Tyr-Ala-(A³)-(A⁴)-Arg-Phe wherein A³ is Ala or Ser and A⁴ is Ala or Pro (SEQ ID NO: 39), wherein the peptide is at least eight amino acids in length.

2. The peptide of claim 1, wherein the peptide has anti-FGF-5 expressing or over-expressing neoplasm biological activity.

3. The peptide of claim 1, wherein the peptide comprises amino acids 3-9 of SEQ ID No: 26.

4. The peptide of claim 1, wherein the peptide comprises an amino acid sequence set forth as SEQ ID NO: 26; SEQ ID NO: 28, SEQ ID NO: 29, SEQ ID NO: 30, or SEQ ID NO: 31.

5. The peptide of claim 4, wherein the peptide consists of an amino acid sequence set forth as SEQ ID NO: 26; SEQ ID NO: 28, SEQ ID NO: 29, SEQ ID NO: 30, or SEQ ID NO: 31.

6. The peptide of claim 1, wherein the peptide is at least 9 amino acids in length.

7. The peptide of claim 1, wherein the peptide is no more than 20 amino acids in length.

8. The peptide of claim 1, wherein the peptide is 8-12 amino acids in length.

9. A purified immunogenic peptide comprising at least 78% sequence identity to SEQ ID NO: 26, wherein the peptide is at least nine amino acids in length.

10. A purified immunogenic peptide comprising at least 80% sequence identity to SEQ ID NO: 32, wherein the peptide is at least 10 amino acids in length.

11. The purified immunogenic peptide of claim 10, wherein the peptide consists of SEQ ID NO: 32.

12. An isolated nucleic acid molecule encoding the peptide of claim 1.

13. A vector comprising the isolated nucleic acid molecule of claim 12.

14. A pharmaceutical composition, comprising a therapeutically effective amount of the peptide of claim 1.

15. A pharmaceutical composition, comprising a therapeutically effective amount of the peptide of claim 10.

16. A pharmaceutical composition, comprising a therapeutically effective amount of a nucleic acid molecule of claim 12.

17. The pharmaceutical composition of claim 14, further comprising a pharmaceutically acceptable carrier.

18. The pharmaceutical composition of claim 17, wherein the pharmaceutically acceptable carrier comprises an adjuvant.

19. A method of eliciting an immune response in a subject against an FGF-5 HLA-A3 epitope, comprising administering to the subject a first dose of a therapeutically effective amount of the peptide of claim 1, resulting in elicitation of the immune response against the FGF-5 HLA-A3 epitope.

20. A method of eliciting an immune response in a subject against an FGF-5 HLA-A2 epitope, comprising administering to the subject a first dose of a therapeutically effective amount of the peptide of claim 10, resulting in elicitation of the immune response against the FGF-5 HLA-A2 epitope.

21. A method of eliciting an immune response in a subject, comprising administering to the subject a therapeutically effective amount of a first dose of the nucleic acid molecule of claim 12.

22. The method of claim 19, wherein an HLA haplotype of the subject is determined prior to administering to the subject a therapeutically effective amount of the peptide, wherein if the subject has an HLA-A3 haplotype, the subject is administered a therapeutically effective amount of the peptide.

23. The method of claim 20, wherein an HLA haplotype of the subject is determined prior to administering to the subject a therapeutically effective amount of the peptide, wherein if the subject has an HLA-A2 haplotype, the subject is administered a therapeutically effective amount of the peptide.

24. The method of claim 19, further comprising determining whether the subject has an FGF-5 expressing neoplasm.

25. The method of claim 19, wherein the subject has an FGF-5 expressing neoplasm, and the elicitation of the immune response stimulates a cytotoxic T cell response against cells of the neoplasm, thereby treating the neoplasm.

26. The method of claim 25, wherein the neoplasm expressing FGF-5 is an adenocarcinoma.

27. The method of claim 25, wherein the neoplasm expressing FGF-5 is a prostate carcinoma, a breast carcinoma, a bladder carcinoma, a pancreas carcinoma, or a renal cell carcinoma (RCC).

28. The method of claim 27, wherein the adenocarcinoma is a renal cell carcinoma (RCC).

29. The method of claim 22, wherein the peptide administered comprises SEQ ID NO: 26; SEQ ID NO: 28, SEQ ID NO: 29, SEQ ID NO: 30, or SEQ ID NO: 31.

30. The method of claim 22, further comprising administering a therapeutically effective amount of one or more other anti-neoplastic compounds

31. The method of claim 30, wherein the one or more other anti-neoplastic compounds comprise IL-2.

32. The method of claim 19, further comprising administering a second dose of therapeutically effective amount of the peptide of claim 1 at a time after the first dose.

33. A method of treating an FGF-5 expressing tumor in a subject, comprising administering to the subject a therapeutically effective amount of the peptide of claim 1, thereby treating the FGF-5 expressing tumor in the subject.

34. The method of claim 33, wherein treatment of the FGF-5 expressing tumor results in a regression of the tumor.

35. A method of generating antibodies specific for an FGF-5 antigen, comprising introducing into a subject the peptide of claim 1.

36. A method of eliciting an immune response in a subject against an FGF-5 HLA-A3 epitope, comprising administering to the subject a first dose of a therapeutically effective amount of immunoreactive sensitized T cells sensitized with the peptide of claim 1, resulting in elicitation of the immune response against the FGF-5 HLA-A3 epitope.

37. A method of eliciting an immune response in a subject against an FGF-5 HLA-A2 epitope, comprising administering to the subject a first dose of a therapeutically effective amount of immunoreactive sensitized T cells sensitized with the peptide of claim 10, resulting in elicitation of the immune response against the FGF-5 HLA-A2 epitope.

38. The method of claim 36, wherein the immunoreactive sensitized T cells sensitized with FGF-5 are autologous or heterologous.

39. A method of stimulating a cytotoxic T cell response against a RCC, comprising:
administering to a subject having RCC a therapeutically effective amount of SEQ ID NO: 26 or 32 sufficient to stimulate the T cell to react with a cell of the RCC.
